(19) United States
(12) Patent Application Publication
(10) Pub. No.: US 2017/0114060 A1
(43) Pub. Date: Apr. 27, 2017

(54) NOVEL EFFECTIVE ANTIVIRAL COMPOUNDS AND METHODS USING SAME

(71) Applicant: THE TRUSTEES OF THE UNIVERSITY OF PENNSYLVANIA, Philadelphia, PA (US)

(72) Inventors: Bruce D. FREEDMAN, Merion Station, PA (US); Ronald N. HARTY, Bensalem, PA (US); Allen B. REITZ, Lansdale, PA (US); Jay E. WROBEL, Lawrenceville, NJ (US)

(21) Appl. No.: 15/315,038

(22) PCT Filed: Jun. 3, 2015
(86) PCT No.: PCT/US15/33975

§ 371 Date: Nov. 30, 2016

Related U.S. Application Data
(60) Provisional application No. 62/007,129, filed on Jun. 3, 2014.
(60) Provisional application No. 62/022,938, filed on Jul. 10, 2014.

(51) Int. Cl. C07D 473/34; A61K 45/06; A61K 31/455; A61K 31/497; C07D 231/12; A61K 31/415; C07D 413/12; A61K 31/422; C07D 401/12; A61K 31/4439; C07D 409/12; A61K 31/4155; C07D 417/12; A61K 31/433; C07D 471/04; A61K 31/437; C07D 401/14; A61K 31/444; C07D 233/64; A61K 31/4164; C07C 233/81; A61K 31/167; C07D 213/81; A61K 31/44; C07C 233/66; C07D 333/38; A61K 31/381; C07D 277/46; A61K 31/426; C07D 417/04; C07D 235/10; A61K 31/4184; C07D 403/04; C07D 417/14; A61K 31/506; C07D 295/192; A61K 31/5375; C07D 403/12; A61K 31/4178; A61K 31/4192; C07D 473/04; A61K 31/69

(52) U.S. Cl. CPC C07D 473/34; A61K 31/69; A61K 45/06; A61K 31/455; A61K 31/497; C07D 231/12; A61K 31/415; C07D 413/12; A61K 31/422; C07D 401/12; A61K 31/4439; C07D 409/12; A61K 31/4155; C07D 417/12; A61K 31/433; C07D 471/04; A61K 31/437; C07D 401/14; A61K 31/444; C07D 233/64; A61K 31/4164; C07C 233/81; A61K 31/167; C07D 213/81; A61K 31/44; C07C 233/66; C07D 333/38; A61K 31/381; C07D 277/46; A61K 31/426; C07D 417/04; C07D 235/10; A61K 31/4184; C07D 403/04; C07D 417/14; A61K 31/506; C07D 295/192; A61K 31/5375; C07D 403/12; A61K 31/4178; A61K 31/4192; C07D 473/04

(57) ABSTRACT

The present invention includes compounds that are useful in preventing or treating viral infections caused by an enveloped RNA virus, such as viral infections caused by a Filovirus, arenavirus, rhabdovirus, paramyxovirus, orthomyxovirus and/or retrovirus. The present invention further includes compositions comprising such compounds, and methods of treating a viral infection in a subject using such compounds.

### CROSS-REFERENCE TO RELATED APPLICATIONS

The present application claims priority under 35 U.S.C. §119(e) to U.S. Provisional Patent Applications No. 62/007,129, filed Jun. 3, 2014, and No. 62/022,938, filed Jul. 10, 2014, all of which applications are incorporated herein by reference in their entireties.

### STATEMENT REGARDING FEDERALLY SPONSORED RESEARCH OR DEVELOPMENT

This invention was made with government support under grant numbers AI1060921, AI1060921 and AI102104 awarded by the National Institute of Allergy and Infectious Diseases (National Institutes of Health). The government has certain rights in the invention.

### BACKGROUND OF THE INVENTION

Filoviruses, such as Ebola (“EBOV”) and Marburg (“MARV”), arenaviruses, such as Lassa fever (“LFV”) and Junin (“JUNV”), and rhabdoviruses, such as vesicular stomatitis virus (“VSV”) and rabies virus (“RABV”), are enveloped RNA viruses that can cause severe disease in humans and animals. For example, filovirus and arenavirus infections can result in hemorrhagic syndromes with high mortality rates in humans, and these viruses are classified as NIAID Category A priority pathogens (Feldmann, et al., 1996. Filoviruses. In Baron S (ed.), Medical Microbiology, 4^{th} Ed, Galveston (Tex.); Feldmann, et al., 1996, Adv. Virus Res. 47:1-52; Grant, et al., 2012, Viruses 4:2317-2339; Peters, et al., 1989, Rev. Infect. Dis. 11 Suppl 4:S743-749). There are currently no approved vaccines or therapeutics to control infection and transmission of EBOV, MARV, LFV, and JUNV.

Outbreaks of these viral infections are typically geographically contained; however, as the recent and catastrophic outbreak of EBOV in West Africa and its unprecedented arrival in the United States illustrated, these viruses are truly global pathogens that are only an “airplane ride” away from establishing infections and potential outbreaks in any country of the world. Fortunately, the incubation period is short and transmission is limited to contact with blood or secretions from infected individuals. However, new variants continue to emerge, and mutations altering the virulence could radically change the characteristics of disease pathogenesis. Thus, new approaches are needed to control transmission of existing and emergent strains. One of the principle challenges to developing antiviral therapies or vaccines is that high mutation rates enable viruses like Ebola and Marburg to evade both (a) compounds directed against viral encoded proteins and functions, and (b) immune regulation by changing epitopes recognized by the host immune system.

As obligate intracellular pathogens, viruses have adopted mechanisms of replication and transmission that critically depend upon host proteins. For EBOV and MARV, the viral matrix protein VP40 orchestrates virion assembly and egress by hijacking host ESCRT proteins including Nedd4 and Tsg101. In fact, EBOV or MARV VP40 expression in host cells is sufficient to coordinate production of authentic viral-like particles (VLPs). Studies of VP40 VLP production have provided critical insight into mechanisms of viral assembly and budding and host proteins involved in virus transmission.

Calcium signals regulate nearly every imaginable host function and are highly conserved among cells. While Ca²⁺ entry in excitable tissues (such as neurons and muscle) is controlled largely by voltage gated calcium channels, one of the principal and functionally critical calcium signaling mechanisms in non-excitable cells is store-operated calcium due to activation of calcium release activated calcium (CRAC) channels, encoded by members of the Orai family of genes. This channel is wholly distinct from voltage activated/dependent calcium channels (VDCCs) and transient receptor potential (TRP) family members including store operated variants. The trigger for Orai activation, stromal interaction molecule 1 or STIM1, is a single pass endoplasmic reticulum membrane embedded transmembrane protein. The N-terminal EF hand domain of STIM1 senses ER calcium levels, and a cytoplasmic C-terminal domain bridges the cytoplasm and directly engages N and C-terminal cytoplasmic tails of Orai in the plasma membrane. When the ER Ca²⁺ concentration drops below its K_{D} (400 μM-600 μM), STIM1 undergoes an N-terminal conformational change leading to its oligomerization, localization within ER domains opposed to the plasma membrane, and physical interaction with and activation of Orai. CRAC channels are encoded by the Orai family of proteins (Orai1, Orai2 and Orai3) and support sustained extracellular calcium entry required for cell functions ranging from gene transcription and subcellular trafficking to cell motility. STIM1/Orai activation is classically triggered by IP3, which binds to and activates receptors (IP3Rs) in the ER membrane through which Ca²⁺ exits the ER to the cytoplasm.

There is an unmet need for the identification and development of potent, broad-spectrum antiviral agents, particularly those that can target high priority pathogens for which no current treatments are available. In particular, there is an unmet need for the development of safe and effective therapeutics against biodefense and high priority viral pathogens, including filoviruses (e.g., Ebola and Marburg) and arenaviruses (e.g., Lassa fever and Junin), which cause severe hemorrhagic fever syndromes with high mortality rates. The present invention addresses and meets these needs.

### BRIEF SUMMARY OF THE INVENTION

The invention provides a method of treating or preventing a viral infection in a subject in need thereof.

The invention further includes a compound of formula (I), or a salt or solvate thereof:

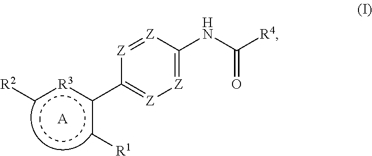

wherein in (I) ring A is a monocyclic or bicyclic cycloalkyl, heterocyclyl, aryl or heteroaryl ring; R¹ and R² are independently selected from the group consisting of optionally substituted C₁-C₁₀ alkyl, optionally substituted C₃-C₁₀ cycloalkyl, optionally substituted C₆-C₁₀ aryl, optionally substituted C₄-C₁₀ heterocyclyl, optionally substituted heteroaryl, halogen, —OH, alkoxy, —NH₂, —N(CH₃)₂, trifluoromethyl, —C≡N, —C(═O)OH, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂; R³ is CH, N, O or S; each occurrence of Z is independently CH or N; and R⁴ is optionally substituted C₃-C₁₀ cycloalkyl, optionally substituted C₆-C₁₀ aryl, optionally substituted C₄-C₁₀ heterocyclyl, or optionally substituted heteroaryl.

In certain embodiments, the method comprises administering to subject an effective amount of at least one inhibitor of a channel selected from the group consisting of calcium-release activated calcium (CRAC) channel and transient receptor potential mucolipin I (TRPML1) channel, whereby the viral infection is treated or prevented in the subject. In other embodiments, administration of the inhibitor blocks, inhibits or interferes with viral spread and/or trafficking within the subject. In yet other embodiments, administration of the inhibitor blocks, inhibits or interferes with viral budding within the subject. In yet other embodiments, administration of the inhibitor blocks, inhibits or interferes with virus dissemination within the subject and/or from the subject to another subject. In yet other embodiments, administration of the inhibitor blocks, inhibits or interferes with viral disease progression in the subject and/or viral disease transmission in the subject or to another subject.

In certain embodiments, the virus is selected from the group consisting of filoviruses, arenaviruses, rhabdoviruses, paramyxoviruses, retroviruses, orthomyxoviruses, and any combinations thereof. In other embodiments, the virus is selected from the group consisting of Influenza A, Influenza B, Influenza C, Junin, Ebola, Marburg, Lassa fever, rabies, vesicular stomatitis, emerging lyssavirus, Nipah, Hendra, HIV-1, HIV-2, HTLV-1, and any combinations thereof.

In certain embodiments, the inhibitor, or a salt or solvate thereof, is at least one selected from the group consisting of: lanthanides; N-(4-(3,5-bis(trifluoromethyl)-1H-pyrazol-1-yl)phenyl)-4-methyl-1,2,3-thiadiazole-5-carboxamide (Pyr2/BTP2/YM58483); ethyl 1-(4-(2,3,3-trichloroacrylamido)phenyl)-5-(trifluoromethyl)-1H-pyrazole-4-carboxylate (Pyr3); N-(4-(3,5-bis(trifluoromethyl)-1H-pyrazol-1-yl)phenyl)-3-fluoroisonicotinamide (Pyr6); N-(4-(3,5-bis(trifluoromethyl)-1H-pyrazol-1-yl)phenyl)-4-methylbenzenesulfonamide (Pyr10); 2-aminoethoxydiphenylborate (2-APB); 2,2′-((((oxybis(methylene))bis(3,1-phenylene))bis(phenylboranediyl))bis(oxy)) bis(ethan-1-amine) (DPB162-AE); 2,2′-((((oxybis(methylene))bis(4,1-phenylene))bis(phenylboranediyl))bis(oxy))bis (ethan-1-amine) (DPB163-AE); capsaicin; 5-nitro-2-(3-phenylpropylamino)-benzoic acid (NPPB); diethylstilbestrol (DES); bromenol lactone (BEL); CM2489; CM3457; cholestatic bile acids; 1-(5-chloronaphthalene-1-sulfonyl)homopiperazine (ML-9); 2,6-difluoro-N-{5-[4-methyl-1-(5-methyl-thiazol-2-yl)-1,2,5,6-tetrahydro-pyridin-3-yl]-pyrazin-2-yl}-benzamide (R02959); N-(2′,5′-dimethoxy-[1,1′-biphenyl]-4-yl)-3-fluoroisonicotinamide (Synta-66 or Synta66); 2,6-difluoro-N-(1-(2-phenoxybenzyl)-1H-pyrazol-3-yl)benzamide (GSK-5503A); 2,6-difluoro-N-(1-(4-hydroxy-2-(trifluoromethyl)benzyl)-1H-pyrazol-3-yl)benzamide (GSK-7975A); 4-[3-(diphenylmethyl)-1,2,4-oxadiazol-5-yl]piperidineyl]piperidine (FCC2121); 3-(4-methyl-1,5-diphenyl-1H-pyrazol-3-yl)-2-phenylpropanoic acid (FCC2122); N-[1-({2-Chloro-5-[(cyclopropylmethyl)oxy]phenyl}methyl)-1H-pyrazol-3-yl]-2,6-difluorobenzamide; N-{1-[(2,4-Dichlorophenyl)methyl]-1H-pyrazol-3-yl}-2,6-difluorobenzamide; 2-Bromo-N-{1-[(2,4-dichlorophenyl)methyl]-1H-pyrazol-3-yl}-6-fluorobenzamide; 2-Chloro-N-{1-[(2,4-dichlorophenyl)methyl]-1H-pyrazol-3-yl}-6-fluorobenzamide; 2,6-Dichloro-N-{1-[(2,4-dichlorophenyl)methyl]-1H-pyrazol-3-yl1 benzamide; N-{1-[(2,4-dichlorophenyl)methyl]-1H-pyrazol-3-yl}-3,5-difluoro-4-pyridine carboxamide; N41-(15-chloro-2-[(phenylmethyl)oxy]phenyl}methyl)-1H-pyrazol-3-yl]-2,6-25 difluorobenzamide; N-{1-[(2,6-dichlorophenyl)methyl]-1H-pyrazol-3-yl}-2,6-difluoro benzamide; N-[1-({5-chloro-2-[(2-methylpropyl)oxy]phenyl}methyl)-1H-pyrazol-3-yl]-2,6-difluorobenzamide; N-(1-{[2-bromo-5-(methyloxy)phenyl]methyl}-1H-pyrazol-3-yl)-2,6-difluorobenzamide; N-(1-{[5-chloro-2-(methyloxy)phenyl]methyl}-1H-pyrazol-3-yl)-2,6-difluorobenzamide; 2,6-Difluoro-N-(1-{[2-(phenyloxy)phenyl]methyl}-1H-pyrazol-3-yl)benzamide; N-[1-({5-bromo-2-[(phenylmethyl)oxy]phenyl}methyl)-1H-pyrazol-3-yl]-2,6-difluorobenzamide; 2,6-Difluoro-N-[1-({2-[(trifluoromethyl)oxy]phenyl}methyl)-1H-pyrazol-3-yl]benzamide; 2,6-Difluoro-N-(1-{[4-[(phenylmethyl)oxy]-2-(trifluoromethyl) phenyl]methyl}-1H-pyrazol-3-yl)benzamide; N-{1-[(2-Bromo-6-chlorophenyl)methyl]-1H-pyrazol-3-yl}-2,6-difluorobenzamide; 2,6-Difluoro-/V-[1-({2-[(phenylmethyl)oxy]phenyl}methyl)-1H-pyrazol-3-yl]benzamide; N/-[1-({2-chloro-5-[(2-methylpropyl)oxy]phenyl}methyl)-1H-pyrazol-3-yl]-2,6-difluorobenzamide; N-(1-{[4-[(cyclopropylmethyl)oxy]-2-(trifluoromethyl)phenyl]methyl}-1H-pyrazol-3-yl)-2,6-difluorobenzamide; 2,6-Difluoro-N-(1-{[4-iodo-2-(trifluoromethyl)phenyl]methyl}-1H-pyrazol-3-yl)benzamide; 2,6-Difluoro-N-(1-{[4-methyl-2-(trifluoromethyl)phenyl]methyl}-1H-pyrazol-3-yl)benzamide; N-(1-{[4-cyclopropyl-2-(trifluoromethyl)phenyl]methyl}-1H-pyrazol-3-yl)-2,6-difluorobenzamide; 2,6-Difluoro-N-{1-[(4-iodo-2-methylphenyl)methyl]-1H-pyrazol-3-yl}benzamide; N-(1-{[4-chloro-2-(trifluoromethyl)phenyl]methyl}-1H-pyrazol-3-yl)-2,6-difluorobenzamide; 2-Fluoro-N-(1-{[4-iodo-2-(trifluoromethyl)phenyl]methyl}-1H-pyrazol-3-yl)benzamide; 2-Chloro-N-(1-{[4-cyclopropyl-2-(trifluoromethyl)phenyl]methyl]-1H-pyrazol-3-yl) benzamide; N-(1-{[4-cyclopropyl-2-(trifluoromethyl)phenyl]methyl}-1H-pyrazol-3-yl)-2-fluorobenzamide; 2,6-Difluoro-N-(1-{[5-iodo-2-(trifluoromethyl)phenyl]methyl}-1H-pyrazol-3-yl)benzamide; 2,6-Difluoro-N-(1-{[2-fluoro-6-(trifluoromethyl)phenyl]methyl}-1H-pyrazol-3-yl)benzamide; 2,6-Difluoro-N-(1-{[4-hydroxy-2-(trifluoromethyl)phenyl] methyl}-1H-pyrazol-3-yl)benzamide; N-[4-(3,5-dicyclopropyl-1H-pyrazol-1-yl)phenyl]-1H-benzo[d]imidazole-6-carboxamide; N-[4-(3,5-dicyclopropyl-1H-pyrazol-1-yl)phenyl]-1H-benzo[d][1,2,3]triazole-6-carboxamide; N-[4-(3,5-dicyclopropyl-1H-pyrazol-1-yl)phenyl] quinoline-6-carboxamide; N-[4-(3,5-dicyclopropyl-1H-pyrazol-1-yl)phenyl]quinoxaline-6-carboxamide; 2-(1H-benzo[d]imidazol-1-yl)-N-[4-(3,5-dicyclopropyl-1H-pyrazol-1-yl) phenyl] acetamide; 2-(1H-benzo[d][1,2,3] triazol-1-yl)-N-[4-(3,5-dicyclopropyl-1H-pyrazol-1-yl)phenyl] acetamide; N-[4-(3,5-dicyclopropyl-1H-pyrazol-1-yl)phenyl]-2-(1H-indol-3-yl)acetamide; N-[4-(3,5-dicyclopropyl-1H-pyrazol-1-yl)phenyl]-2-(imidazo[1,2-a]pyridin-2-yl) acetamide; N-[4-(3,5-dicyclopropyl-1H-pyrazol-1-yl)phenyl]-2-(quinolin-6-yl)acetamide; N-[4-(3,5-dicyclopropyl-1H-pyrazol-1-yl)phenyl]-2-(quinolin-6-yl)acetamide; 2-(1H-benzo[d][1,2,3]triazol-1-yl)-N-(4-(3,5-dicyclopropyl-1H-pyrazol-1-yl)-3-fluorophenyl) acetamide; N-[4-(3,5-dicyclopropyl-1H-pyrazol-1-yl)-3-fluorophenyl]-2-(quinolin-6-yl) acetamide; N-[6-(3,5-dicyclopropyl-1H-pyrazol-1-yl)pyridin-3-yl]quinoline-6-carboxamide; N-[6-(3,5-dicyclopropyl-1H-pyrazol-1-yl)pyridin-3-yl]quinoxaline-6-carboxamide; 2-(1H-benzo[d] [1,2,3]triazol-1-yl)-N-[6-(3,5-dicyclopropyl-1H-pyrazol-1-yl)pyridin-3-yl]acetamide; N-[6-(3,5-dicyclopropyl-1H-pyrazol-1-yl)pyridin-3-yl]-2-(quinolin-6-yl)acetamide; N-{4-[5-cyclopropyl-3-(trifluoromethyl)-1H-pyrazol-1-yl]phenyl}quinoline-6-carboxamide; N-{4-[5-cyclopropyl-3-(trifluoromethyl)-1H-pyrazol-1-yl]phenyl}quinoxaline-6-carboxamide; 2-(1H-benzo[d]imidazol-1-yl)-N-{4-[5-cyclopropyl-3-(trifluoromethyl)-1H-pyrazol-1-yl] phenyl]acetamide; 2-(1H-benzo[d][1,2,3]triazol-1-yl)-N-{4-[5-cyclopropyl-3-(trifluoromethyl)-1H-pyrazol-1-yl]phenyl}acetamide; 2-(2H-benzo[d][1,2,3]triazol-2-yl)-N-{4-[5-cyclopropyl-3-(trifluoromethyl)-1H-pyrazol-1-yl]phenyl}acetamide; 2-(3H-[1,2,3]triazolo[4,5-b]pyridin-3-yl)-N-{4-[5-cyclopropyl-3-(trifluoromethyl)-1H-pyrazol-1-yl] phenyl}acetamide; (S)-2-(3H-[1,2,3]triazolo[4,5-b]pyridin-3-y])-N-{4-[5-cyclopropyl-3-(trifluoromethyl)-1H-pyrazol-1-yl] phenyl} propanamide; 2-(6-amino-9H-purin-9-yl)-N-{4-[5-cyclopropyl-3-(trifluoromethyl)-1H-pyrazol-1-yl]phenyl}acetamide; N-(4-(5-cyclopropyl-3-(trifluoromethyl)-1H-pyrazol-1-yl)phenyl)-2-(1,3-dimethyl-2,6-dioxo-2,3-dihydro-1H-purin-7(6H)-yl)acetamide; N-{4-[5-cyclopropyl-3-(trifluoromethyl)-1H-pyrazol-1-yl)phenyl)-2-(imidazo[1,2-a] pyridin-2-yl) acetamide; N-{4-[5-cyclopropyl-3-(trifluoromethyl)-1H-pyrazol-1-yl]phenyl}-2-(quinolin-6-yl)acetamide; N-{4-[5-cyclopropyl-3-(trifluoromethyl)-1H-pyrazol-1-yl]phenyl}-2-(quinolin-6-yl)propanamide; N-{4-[5-cyclopropyl-3-(trifluoromethyl)-1H-pyrazol-1-yl]-3-fluorophenyl}-1H-benzo[d][1,2,3] triazole-6-carboxamide; 2-(1H-benzo[d][1,2,3]triazol-1-yl)-N-{4-[5-cyclopropyl-3-(trifluoromethyl)-1H-pyrazol-1-yl]-3-fluorophenyl}acetamide; N-{6-[5-cyclopropyl-3-(trifluoromethyl)-1H-pyrazol-1-yl]pyridin-3-yl}-1H-benzo[d][1,2,3] triazole-5-carboxamide; 2-(1H-benzo[d][1,2,3]triazol-1-yl)-N-{6-[5-cyclopropyl-3-(trifluoromethyl)-1H-pyrazol-1-yl]pyridin-3-yl} acetamide; 2-(2H-benzo[d][1,2,3]triazol-2-yl)-N-{6-[5-cyclopropyl-3-(trifluoromethyl)-1H-pyrazol-1-yl]pyridin-3-yl} acetamide; N-{6-[5-cyclopropyl-3-(trifluoromethyl)-1H-pyrazol-1-yl]pyridin-3-yl}-2-(quinolin-6-yl)acetamide; 2-(1H-benzo[d][1,2,3]triazol-1-yl)-N-{6-[4-chloro-5-cyclopropyl-3-(trifluoromethyl)-1H-pyrazol-1-yl]pyridin-3-yl} acetamide; 4-[5-cyclopropyl-3-(trifluoromethyl)-1H-pyrazol-1-yl]-3-fluoro-N-(quinolin-6-ylmethyl)benzamide; 1-[4-(3,5-dicyclopropyl-1H-pyrazol-1-yl)phenyl]-3-(quinolin-6-yl)urea; 4-[6-(2-chloro-6-fluoro-phenyl)-5H-pyrrolo[3,2-d]pyrimidin-2-yl]-3,N,N-trimethyl-benzenesulfonamide; 6-(2-Chloro-phenyl)-2-(2-methyl-5-trifluoromethyl-2H-pyrazol-3-yl)-5H-pyrrolo[2,3-b]pyrazine; 4-[6-(2-Chloro-phenyl)-5H-pyrrolo[2,3-b]pyrazin-2-yl]-3-methyl-benzoic acid methyl ester; 4-(6-(2-Chlorophenyl)-5H-pyrrolo[2,3-b]pyrazin-2-yl)-N,N,3-trimethylbenzenesulfonamide; 6-(2-chloro-6-fluorophenyl)-2-(1-methyl-3-(trifluoromethyl)-1H-pyrazol-5-yl)-5H-pyrrolo[2,3-b]pyrazine; 6-Cyclohexyl-2-(1-methyl-3-(trifluoromethyl)-1H-pyrazol-5-yl)-5H-pyrrolo[2,3-b]pyrazine; 4-(6-Cyclohexyl-5H-pyrrolo[2,3-b]pyrazin-2-yl)-N,N,3-trimethylbenzenesulfonamide; 2,6-Difluoro-N-(6-(5-(4-methyl-5-oxo-4,5-dihydro-1,3,4-oxadiazol-2-yl)-3-(trifluoromethyl)-1H-pyrazol-1-yl)pyridin-3-yl)benzamide; 2-Fluoro-6-methyl-N-(6-(5-(4-methyl-5-oxo-4,5-dihydro-1,3,4-oxadiazol-2-yl)-3-(trifluoromethyl)-1H-pyrazol-1-yl)pyridin-3-yl)benzamide; 5-(3-Cyclopropyl-1-(5-((2,6-difluorobenzyl)amino)pyridin-2-yl)-1H-pyrazol-5-yl)-3-methyl-1,3,4-oxadiazol-2(3H)-one; N-(6-(3-(Difluoromethyl)-5-(4-methyl-5-oxo-4,5-dihydro-1,3,4-oxadiazol-2-yl)-1H-pyrazol-1-yl)pyridin-3-yl)-2,6-difluorobenzamide; 5-(1-(5-(2,6-Difluorobenzyl)amino)pyridin-2-yl)-5-(fluoromethyl)-1H-pyrazol-3-yl)-3-methyl-1,3,4-oxadiazol-2(3H)-one; Methyl 3-(1-(5-(2,6-difluorobenzyl)amino)pyridin-2-yl)-5-(trifluoromethyl)-1H-pyrazol-3-yl)-5-methyl-4,5-dihydroisoxazole-5-carboxylate; Methyl 3-(1-(5-(2-chloro-6-fluorobenzyl)amino)pyridin-2-yl)-5-(trifluoromethyl)-1H-pyrazol-3-yl)-5-methyl-4,5-dihydroisoxazole-5-carboxylate; 2,6-Difluoro-W-(6-(3-(4-methyl-5-oxo-4,5-dihydro-1,3,4-oxadiazol-2-yl)-5-(trifluoromethyl)-1H-pyrazol-1-yl)pyridin-3-yl)benzamide; 2-Chloro-6-fluoro-N-(6-(3-(4-methyl-5-oxo-4,5-dihydro-1,3,4-oxadiazol-2-yl)-5-(trifluoromethyl)-\H-pyrazol-1-yl)pyridin-3-yl)benzamide; 2-Fluoro-6-methyl-/v-(6-(3-(4-methyl-5-oxo-4,5-dihydro-1,3,4-oxadiazol-2-yl)-5-(trifluoromethyl)-1H-pyrazol-1-yl)pyridin-3-yl)benzamide; N-(6-(5-(Difluoromethyl)-3-(4-methyl-5-oxo-4,5-dihydro-1,3,4-oxadiazol-2-yl)-1H-pyrazol-1-yl)pyridin-3-yl)-2,6-difluorobenzamide; 5-(1-(5-((2,6-Difluorobenzyl) amino)pyridin-2-yl)-5-(difluoromethyl)-1H-pyrazol-3-yl)-3-methyl-1,3,4-oxadiazol-2(3H)-one; 5-(1-(5-(2,6-Difluorobenzyl)amino)pyridin-2-yl)-3-(difluoromethyl)-1H-pyrazol-5-yl)-3-methyl-1,3,4-oxadiazol-2(3H)-one; N-(6-(3-(5,5-Dimethyl-4-oxo-4,5-dihydroisoxazol-3-yl)-5-(trifluoromethyl)-1H-pyrazol-1-yl)pyridin-3-yl)-2,6-difluorobenzamide; 2-Chloro-N-(6-(3-(5,5-dimethyl-4-oxo-4,5-dihydroisoxazol-3-yl)-5-(trifluoromethyl)-1H-pyrazol-1-yl)pyridin-3-yl)-6-fluorobenzamide; 2,6-Difluoro-N-(6-(1′,4′,4,-trimethyl-5′-oxo-5-(trifluoromethyl)-4′,5′-dihydro-1H,1H′-[3,3′-bipyrazol]-1-yl)pyridin-3-yl)benzamide; 2-Chloro-6-fluoro-N-(6-(1,4,4′-trimethyl-5′-oxo-5-(trifluoromethyl)-4′,5,-dihydro-1H,1′H-[3,3′-bipyrazol]-1-yl)pyridin-3-yl)benzamide; 2-Fluoro-6-methyl-N-(6-(1,4,4′-trimethyl-5′-oxo-5-(trifluoromethyl)-4′,5′-dihydro-1H,1′H-[3,3′-bipyrazol]-1-yl)pyridin-3-yl)benzamide; 2,6-Difluoro-N-(6-(3-(4-methyl-5-oxo-4,5-dihydro-1,2,4-oxadiazol-3-yl)-5-(trifluoromethyl)-1H-pyrazol-1-yl)pyridin-3-yl)benzamide; N-(6-(3-(4-Acetyl-5,5-dimethyl-4,5-dihydro-1,3,4-oxadiazol-2-yl)-5-trifluoromethyl)-1H-pyrazol-1-yl)pyridin-3-yl)-2,6-difluorobenzamide; N-(6-(3-(4,4-Dimethyl-4,5-dihydrooxazol-2-yl)-5-(trifluoromethyl)-1H-pyrazol-1-yl)pyridin-3-yl)-2,6-difluorobenzamide; 5-(1-(5-(2,6-Difluorobenzyl)amino)pyridin-2-yl)-5-(trifluoromethyl)-1H-pyrazol-3-yl)-3-methyl-1,3,4-oxadiazol-2(3H)-one; 5-(1-(5-((2-Chloro-6-fluorobenzyl)amino)pyridin-2-yl)-5-(trifluoromethyl)-1H-pyrazol-3-yl)-3-methyl-1,3,4-oxadiazol-2(3iy)-one; 1′454(2,6-Difluorobenzyl)amino)pyridin-2-yl)-1,4,4-trimethyl-5′-(trifluoromethyl)-1H,1′H-[3,3′-bipyrazol]-5(4H)-one; 1′-(5-(2-Chloro-6-fluorobenzyl)amino)pyridin-2-yl)-1,4,4-trimethyl-5′-(trifluoromethyl)-1H,1′H-[3,3′-bipyrazol]-5(4H)-one; 3-(1-(5-((2,6-Difluorobenzyl) amino)pyridin-2-yl)-5-(trifluoromethyl)-1H-pyrazol-3-yl)-4-methyl-1,2,4-oxadiazol-5(4H)-one; 1-(5-(1-(5-(2,6-Difluorobenzyl)amino)pyridin-2-yl)-5-(trifluoromethyl)-1H-pyrazol-3-yl)-2,2-dimethyl-1,3,4-oxadiazol-3(2H)-yl)ethanone; N-(2,6-Difluorobenzyl)-6-(3-(4,4-dimethyl-4,5-dihydrooxazol-2-yl)-5-(trifluoromethyl)-1H-pyrazol-1-yl)pyridin-3-amine; N-(6-(5-Cyclopropyl-3-(4-methyl-5-oxo-4,5-dihydro-1,3,4-oxadiazol-2-yl)-1H-pyrazol-1-yl)pyridin-3-yl)-2,6-difluorobenzamide; N-(6-(3-Cyclopropyl-5-(4-methyl-5-oxo-4,5-dihydro-1,3,4-oxadiazol-2-yl)-1H-pyrazol-1-yl)pyridin-3-yl)-2,6-difluorobenzamide; 2,6-Difluoro-N-(6-(5-methyl-3-(4-methyl-5-oxo-4,5-dihydro-1,3,4-oxadiazol-2-yl)-1H-pyrazol-1-yl)pyridin-3-yl)benzamide; 5-(1-(5((2,6-Difluorobenzyl)amino)pyridin-2-yl)-5-methyl-1H-pyrazol-3-yl)-3-methyl-1,3,4-oxadiazol-2(3H)-one; (3-(1-(5-((2,6-Difluorobenzyl)amino) pyridin-2-yl)-5-(trifluoromethyl)-1H-pyrazol-3-yl)-5-methyl-4,5-dihydroisoxazol-5-yl)methanol; (3-(1-(5-(2-Chloro-6-fluorobenzyl)amino)pyridin-2-yl)-5-(trifluoromethyl)-1H-pyrazol-3-yl)-5-methyl-4,5-dihydroisoxazol-5-yl)methanol; Methyl 3-(1-(5-(2,6-difluorobenzamido)pyridin-2-yl)-5-(trifluoromethyl)-1H-pyrazol-3-yl)-5-methyl-4,5-dihydroisoxazole-5-carboxylate; 2,6-Difluoro-N-(6-(3-(5-(hydroxymethyl)-5-methyl-4,5-dihydroisoxazol-3-yl)-5-(trifluoromethyl)-1H-pyrazol-1-yl)pyridin-3-yl)benzamide; 3-(1-(5-(2,6-Difluorobenzamido) pyridin-2-yl)-5-(trifluoromethyl)-1H-pyrazol-3-yl)-5-methyl-4,5-dihydroisoxazole-5-carboxamide; 2,6-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Chloro-6-fluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Fluoro-6-methyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Fluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,3-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,4,5-Trifluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,3,4-Trifluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,4-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,3-Dimethyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Chloro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Methyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 4-Ethyl-N-(5-(3-(4-methyl-5-oxo-4,5-dihydro-1,3,4-oxadiazol-2-yl)-5-(trifluoromethyl)-1//-pyrazol-1-yl)pyridin-2-yl)benzamide; N-(5-(3-(4-Methyl-5-oxo-4,5-dihydro-13,4-oxadiazol-2-yl)-5-(trifluoromethyl)-1H-pyrazol-1-yl)pyridin-2-yl)-2-naphthamide; 5-(1-(6((2,6-Difluorobenzyl)amino)pyridin-3-yl)-5-(trifluoromethyl)-1H-pyrazol-3-yl)-3-methyl-1,3,4-oxadiazol-2(3H)-one; 5-(1-(6-((2-Chloro-6-fluorobenzyl)amino)pyridin-3-yl)-5-(trifluoromethyl)-1H-pyrazol-3-yl)-3-methyl-1,3,4-oxadiazol-2(3H)-one; 5-(1-(6-(2-Fluoro-6-methylbenzyl)amino)pyridm-3-yl)-5-(trifluoromethyl)-1H-pyrazol-3-yl)-3-methyl-1,3,4-oxadiazol-2(3H)-one; N-(2,6-Difluorophenyl)-6-(3-(4-methyl-5-oxo-4,5-dihydro-1,3,4-oxadiazol-2-yl)-5-(trifluoromethyl)-1H-pyrazol-1-yl)nicotinamide; N-(2-Chloro-6-fluorophenyl)-6-(3-(4-methyl-5-oxo-4,5-dihydro-1,3,4-oxadiazol-2-yl)-5-(trifluoromethyl)-1H-pyrazol-1-yl)nicotinamide; 2-(4-Chloro-phenyl)-3-[1-(4-chloro-phenyl)-5-methyl-1H-pyrazol-3-yl]-propionic acid (FC-2399); the compounds illustrated in FIGS. 21A-21D, FIG. 22, FIG. 23, and FIGS. 24A-24I; the compounds listed in Table 1; a compound of formula (I)

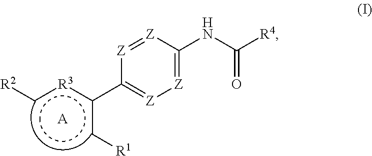

wherein in (I): ring A is a monocyclic or bicyclic cycloalkyl, heterocyclyl, aryl or heteroaryl ring; R¹ and R² are independently selected from the group consisting of optionally substituted C₁-C₁₀ alkyl, optionally substituted C₃-C₁₀ cycloalkyl, optionally substituted C₆-C₁₀ aryl, optionally substituted C₄-C₁₀ heterocyclyl, optionally substituted heteroaryl, halogen, —OH, alkoxy, —NH₂, —N(CH₃)₂, trifluoromethyl, —CN, —C(═O)OH, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂; R³ is CH, N, O or S; each occurrence of Z is independently CH or N; and R⁴ is optionally substituted C₃-C₁₀ cycloalkyl, optionally substituted C₆-C₁₀ aryl, optionally substituted C₄-C₁₀ heterocyclyl, or optionally substituted heteroaryl.

In certain embodiments, the inhibitor is administered as part of a pharmaceutical composition. In other embodiments, the subject is further administered at least one additional antiviral agent. In yet other embodiments, the agent and the inhibitor are co-administered to the subject. In yet other embodiments, the agent and the inhibitor are co-formulated. In yet other embodiments, the subject is a mammal. In yet other embodiments, the mammal is human.

In certain embodiments, the compound contemplated within the invention is in a pharmaceutical composition. In other embodiments, the pharmaceutical composition further comprises at least one additional antiviral agent. In yet other embodiments, the compound and antiviral agent are coformulated.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates a model of interplay between VP40 and Ca²⁺. STIM1 and Orai1 signaling (right) are required for VLP formation. In certain embodiments, VP40 activation of host IP3R, induction of reactive oxygen species (ROS), or regulation of SERCA activity may play a role in VP40-mediated Ca²⁺ mobilization. In other embodiments, VP40-induced Ca²⁺ signals play a role in Alix activation, interactions with VP40 or other ESCRT proteins, membrane localization and TRPML1 activation via ALG-2.

FIG. 2 comprises a set of graphs illustrating calcium release from ER and entry via Oral, and illustrating that 2-APB and genetic inactivation of Orai1 similarly block store operated Ca²⁺ influx in HEK293T cells used in these studies.

FIG. 3 comprises a series of graphs illustrating the finding that VP40 expression mobilizes cytoplasmic Ca²⁺. Ebola or Marburg VP40 or Junin virus Z protein was expressed in HEK 293T cells co-expressing genetically encoded Ca²⁺ indicator R-GECO-1. Fluorescence emission (580 nM) of R-GECO-1 was monitored between 6 and 24 hours post transfection (top line) in cells in an environmentally controlled chamber on the stage of a Yokagawa spinning disk confocal microscope. Expression of the VP40 matrix protein of Ebola Virus (EBOV), the VP40 matrix protein of Marburg Virus (MARV), and the Z matrix protein of Junin Virus (JUNV) results in an Orai-dependent increase in intracellular calcium levels over 24 hours (line 1). A small cytoplasmic Ca²⁺ elevation was induced by a control GFP plasmid (line 2). This demonstrates that EBOV, MARV, and JUNV matrix protein trigger an increase in cellular calcium in target cells. eVP40, mVP40, and JUNV Z and control vector failed to mobilize Ca²⁺ in E106A HEK 293T cells: lines 3 show no increase in intracellular calcium levels in E106A cells that express an inactive dominant negative mutant of Orai; lines 4 represent a vector alone negative control in E106A cells. Each line represents the average±SEM of at least 20 cells at least 3 expts).

FIG. 4 comprises a set of graphs illustrating the inhibition of Ca²⁺ entry via CRAC channels by 2-APB and Synta 66 in HEK293T cells.

FIGS. 5A-5B illustrates the finding that Ebola, Marburg, Lassa Fever, and Junin VLP budding is reduced in E106A cells. FIG. 5A: Left panes—series of images illustrating inhibition of VP40 VLP production in cells (E106A cells) expressing a dominant negative mutant of Orai1. VP40 measured in VLPs from WT & E106A cells (lane 2 vs. lane 4). Actin expression, used as a control, demonstrates the specificity of these drugs. Right panes—series of images illustrating inhibition of VP40 VLP production by Orai1 inactivation in Marburg VP40. VP40 measured in VLPs from WT & E106A cells (lane 2 vs. lane 4). Actin expression, used as a control, demonstrates the specificity of these drugs. These data demonstrate that a functional Orai1 channel is required for efficient egress of EBOV and MARV VP40 VLPs. FIG. 5B is a set of images illustrating results relating to Orai regulation of Junin virus and Lassa fever VLP formation. The pathogenesis of Junin (JUNV) and Lassa Fever (LF) viruses is similar to that of Ebola and Marburg viruses, and they are also classified as NIAID Category A agents. As the arenavirus Z protein is functionally homologous to VP40 in orchestrating VLP assembly and egress, budding of JUNV and LFV VLPs was inhibited from E106A cells. These results indicate that a functional cellular Orai1 channel is critical for efficient budding of these VLPs from four different hemorrhagic fever viruses.

FIG. 6 comprises a set of images illustrating effects of Orai1 inhibitor Synta66 on viral budding. Top panel: 3D reconstructions of confocal images illustrating that the Orai1 inhibitor Synta66 inhibits VP40-induced membrane protrusions but has no apparent effect on VP40 expression or membrane localization. Bottom panel: a single confocal cross section of HEK293T cells showing comparable membrane localization of eVP40 GFP in WT and Synta66 treated cells. Together, these results indicate that Ca²⁺ entry via Orai controls late steps of viral budding. eVP40 GFP expressed in HEK cells. Cytoplasm stained with Cell Mask (red).

FIG. 7 illustrates STIM1 knockdown suppresses eVP40 VLP formation and Ebola VLP budding depends on host STIM1. Knockout of STIM1 by using either siRNAs (panels on top right and bottom right) or shSTIM1 plasmids (panel on bottom left) results in reduced Ebola VP40 (eVP40) VLP budding. Importantly, re-expression of STIM1 rescues budding of eVP40 VLPs (both bottom panels). In the bottom left panel, cells were transfected with DNA plasmids that express an shRNA targeted to the 5′-untranslated region of STIM1 (middle row) or a bicistronic plasmid that expresses this same shRNA and also expresses full length STIM1 cDNA to rescue STIM1 expression, illustrating suppression (middle row) and rescue (bottom row) of VLP production by STIM1. The data demonstrate that expression of cellular STIM1 is important for efficient budding of Ebola VP40 VLPs.

FIG. 8A, comprising a set of images and bar graphs, illustrates the finding that pharmacologic inhibition of Orai by small molecule compounds 2-APB or Synta66 reduces Ebola and Marburg VLP budding by up to 100-fold at 50 μM concentrations. These concentrations of 2-APB and Synta66 were not cytotoxic to the cells under these experimental conditions as determined by MTT cell viability assays (bar graphs). FIG. 8B illustrates that R02959 (5 μM) inhibits Ca²⁺ entry via CRAC channels and exerts a dose dependent inhibition of eVLP formation.

FIG. 9 comprises a set of graphs and images illustrating the finding that novel 2-APB related compound FC-2122 inhibits Orai-mediated calcium entry and eVP40-mediated VLP production with a similar dose dependence.

FIG. 10 comprises a set of images illustrating that pharmacologic inhibition of Orai by Synta66 and 2-APB reduces live Junin Virus (Candid-1 strain) production in vitro in a dose-dependent and statistically significant manner. Top panel: Synta66 reduces the number of JUNV foci in a dose-dependent manner (left), without effect on the viability of cells under conditions mimicking those used for infection experiments (right). Bottom panel: 2-APB treatment of infected cells induced a similar dose-dependent decrease in JUNV budding (left), without effect on the viability of cells under conditions mimicking those used for infection experiments (right). Expression of the Junin GP protein in untreated and treated cells is shown by Western blot and demonstrates that viral protein expression is unaffected by these compounds at the indicated concentrations.

FIG. 11A comprises a graph illustrating the finding that release of VSV virus from E106A cells decreased as compared to WT 293T cells. The graph illustrates titers of VSV as a function of time, and the image illustrates that time-dependent VSV titers from 6, 8, and 12 hours post-infection were reduced by approximately 10-fold (1 log) compared to those from WT HEK293T cells. FIG. 11B illustrates the finding that a dose-dependent inhibition of VSV budding by 2-APB was evident. However, 2-APB treatment had little to no effect on cellular expression of VSV M protein. FIG. 11C illustrates the finding that Synta66 also inhibited VSV titers without affecting expression of VSV M or cellular HSP 70 protein expression. These results correlate well with those described for filoviruses and arenaviruses, and suggest that Orai1 function is crucial for efficient egress of live VSV from a cell culture model of infection.

FIG. 12 comprises a graph illustrating dose-dependent inhibition of live BSL-4 pathogen (Lassa, Junin, Marburg and Ebola) budding and spread in vitro by the Orai selective inhibitor Synta66. These data validate the VLP findings and establish a general role for Orai mediated Ca²⁺ entry in live Filovirus and arenavirus transmission and are consistent with effects of Synta66 and 2-APB on live rhabdovirus (VSV) transmission (FIG. 11A-11C).

FIG. 13 comprises a set of images illustrating Orai1 regulation of HIV-1 Gag-mediated VLP formation and suggest that HIV-1 buds by a similar Orai1-dependent mechanism as Filoviruses, arenaviruses and rhabdoviruses.

FIG. 14 comprises a graph illustrating Orai1 regulation of live Influenza A virus budding and transmission, and together with studies of filoviruses, arenaviruses, VSV (rhabdovirus), and HIV-1 (retrovirus) Gag helps establish a general and conserved role for Orai mediated Ca²⁺ entry in the budding or transmission of enveloped RNA viruses.

FIG. 15 comprises a set of images illustrating the finding that TRPML1 knockdown reduces VP40 VLP formation. TRPML1 suppression by siRNA significantly reduced VP40 induced VLP production (lane 2 vs. lane 3) TRPML1 is another Ca²⁺ entry channel and its activity is Ca²⁺ dependent. These data identify TRPML1 as an additional host target of Ca²⁺ regulation and an additional Ca²⁺ channel that represents a target for pharmacological inhibition of enveloped RNA virus budding.

FIG. 16A illustrates the finding that expression of host protein is important for efficient budding of eVP40 VLPs. Knockdown of Alix by siRNAs (lane 3) reduced budding of eVP40 VLPs compared to non-specific control siRNAs (lane 2). Alix is a host protein of interest because its function is known to be regulated by calcium levels. FIG. 16B illustrates the finding that expression of Alix or the Bro1-V fragment of Alix can rescue budding of an L-domain mutant of eVP40. FIG. 16C illustrates the finding that 2-APB sensitivity of Alix Bro1-V rescue of VLP formation demonstrates that Orai1/Ca²⁺ dependence of Alix rescue of VLP. FIG. 16D illustrates the finding that Ca²⁺ activation of full length Alix (but not AlixBro1-V fragment) rescues VLP budding by L-domain mutant VP40, indicating that Ca²⁺ is required for Alix unfolding and activation during VLP formation.

FIG. 17 comprises a non-limiting illustration of the mechanisms by which VP40 and live filovirus may generate cytoplasmic Ca²⁺ signals as well as a potential regulatory role for calcium in VP40-mediated virus like particle formation and budding. Each of these is an additional target for control of enveloped RNA virus budding. Known protein-protein interactions are shown in the boxes.

FIG. 18 comprises a non-limiting illustration of steps of filovirus VP40 induced VLP production and steps of live virus budding that may be controlled by Ca²⁺. In certain embodiments, each of these is an additional target for control of enveloped RNA virus budding.

FIG. 19A illustrates the finding that lifetime of GFP fluorescence (VP40) is reduced upon interaction with mCherry Tsg101 (pseudocolored blue, left panel) around periphery of cell. FIG. 19B illustrates statistical significance of VP40-GFP interactions with mCherry-Tsg101.

FIG. 20 comprises an image illustrating visualization of VP40-mediated VLP formation with TIRF microscopy. GFP-VP40 expression in HEK 293T cells results in tubovesicular plasma membrane protrusions. TIRF image of TVS structures at 70 nm resolution.

FIGS. 21A-21D, FIG. 22, FIG. 23, and FIGS. 24A-24I independently illustrate compounds useful within the methods of the invention.

FIG. 25 illustrates the finding that Reactive Oxygen Species (ROS) inhibitors N-acetyl cysteine (NAC) and NSC 62914 block the Ebola VP40 induced intracellular calcium increase over 24 hours. Line 1 is the positive control as shown above in the top graphs, and line 2 is vector alone as a negative control.

FIG. 26 comprises a bar graph illustrating the finding that the concentrations of Synta66 used in FIG. 12 are not cytotoxic as shown by Alomar blue cell viability assay.

FIG. 27 comprises a set of images illustrating the finding that pharmacologic inhibition of Orai (via Synta66) results in a dose-dependent reduction of live, authentic Ebola, Marburg, Lassa Fever, and Junin Virus production in vitro. Numbers in white are percent infected cells (shown in green/light gray).

FIG. 28 comprises a set of images illustrating the finding that pharmacologic inhibition of Orai (via Synta66) does not result in altered expression of Ebola VP40 (green) at the plasma membrane (left panel). In right panel, the absence of Orai (in E106A cells) results in an increase in cytoplasmic Nedd4 complex accumulation relative to plasma membrane. The fluorescence (Nedd4 expression) is quantified in the bar graph. These results suggest that calcium influx via Orai may be affecting host proteins such as Nedd4 that are involved in efficient budding of eVP40 VLPs.

FIG. 29 comprises a set of electron images illustrating the finding that pharmacologic inhibition of Orai (via Synta66) reduces Ebola VP40-mediated virus-like particle formation at the plasma membrane. Filament-like protrusions represent VP40 VLPs, and these VLP structures are absent in vector alone, and reduced at the plasma membrane of cells treated with Synta66.

FIG. 30, comprising a calcium trace graph, western blots, and bar graphs, illustrates the finding that novel Orai channel inhibitors (FC-2122 and FC-2121) show successful inhibition of Ebola VP40 VLP production in vitro (Western blot) without cell toxicity (bar graphs of MTT assay data). Both FC-2121 (green line) and FC-2122 (red line) can block calcium influx into cells via Orai compared to vehicle alone control (black line).

FIG. 31, comprising a bar graph and western blots, illustrates the finding that suppression of host TRPML1 (calcium channel) expression using siRNA results in a reduction of Ebola VP40 VLP production.

FIG. 32, comprising a bar graph and western blots, illustrates the finding that TRPML1 inhibition (indirectly via compound YM201636 or directly by compound B4) results in a dose-dependent reduction of Ebola VP40 VLP production.

FIG. 33 comprises a schematic model illustrating the potential role of calcium and TRPML1 in VLP production.

FIB. 34 comprises a graph illustrating the finding that novel Orai inhibitors (FC-2121 and FC-2399) reduce Orai-mediated calcium entry in vitro compared to no drug control.

FIG. 35 comprises a set of bar graphs illustrating the finding that novel Orai inhibitors (FC-2121, FC-2122, FC-2399, and FC-2398) reduce live Influenza Virus A budding in vitro from MDCK cells as shown by RT-PCR and the number of copies of the virus mRNA encoding the viral M1 protein.

FIG. 36 comprises a set of bar graph illustrating the finding that compounds FC-2121 and FC-2122 are not cytotoxic to human HEK293T or MDCK (canine) cells that were used for the influenza A virus studies.

FIG. 37 illustrates the finding that novel Orai inhibitor (FC-2399) reduces budding of live HIV-1 in vitro in HEK293T cells as shown by the reduction in viral capsid (CA) protein in budding virus (top panel). No significant changes were observed in the levels of viral proteins expressed in cells. Lanes C1 and C2 are negative control lanes. Compound FC-2122 did not inhibit budding of live HIV-1 at these concentrations (1-10 μM). These results show that Orai inhibitor FC-2399 can block budding of live HIV-1.

FIG. 38 comprises a bar graph illustrating the finding that pharmacologic inhibition of Orai (with Synta66) results in a dose-dependent reduction in budding of live Vesicular Stomatitis Virus in vitro at an MOI of 0.01. The percent reduction in virus titer from five averaged experiments is shown.

FIG. 39 comprises a bar graph illustrating the finding that pharmacologic inhibition of Orai (with Synta66) results in a dose-dependent reduction in budding of live Vesicular Stomatitis Virus in vitro at an MOI of 0.1. The percent reduction in virus titer from five averaged experiments is shown.

FIG. 40 comprises a bar graph illustrating the finding that novel Orai inhibitor (FC-2399) significantly reduced budding of live Vesicular Stomatitis Virus in vitro in a dose-dependent manner in five independent experiments.

### DETAILED DESCRIPTION OF THE INVENTION

The present invention relates in part to the unexpected discovery of a novel host mechanism that is required for effective viral budding by human and animal enveloped RNA pathogens, such as but not limited to, Ebola, Marburg, HIV-1, Influenza A, Vesicular Stomatitis, Lassa Fever, and/or Junin viruses.

In one aspect, the present invention focuses on host mechanisms that critically regulate viral assembly and budding. Without wishing to be limited by any theory, an significant advantage of a host-directed therapeutic approach is that the host is immutable. Consequently, therapeutics that target conserved host pathways used by families of viruses for transmission should provide broader spectrum efficacy than drugs that exclusively target viral proteins or processes, and these host targets should be insensitive to selective pressures that normally allow pathogens to develop drug resistance.

As demonstrated herein, these pathogens have a fundamental requirement for calcium signals generated within host cells during late steps of viral particle transport and/or budding. These signals are generated through a “store operated” calcium (SOC) signaling mechanism within host cells. The molecular components of the store-operated calcium signaling are found in various non-excitable cells and are typically activated via G-protein and tyrosine kinase coupled plasma membrane receptors. The molecular components of this pathway include an ER calcium sensor, STIM1 (and/or STIM2), and the plasma membrane calcium channel Orai1 (and/or Orai2 and Orai3), wherein the Orai family of genes encodes calcium-release activated calcium (CRAC) channels. Specifically, STIM activates Orai1 following IP3-mediated depletion of Ca²⁺ within the ER.

The present results demonstrate that suppression of STIM1 expression and/or genetic inactivation or pharmacological blockade of Orai1 channels inhibit viral budding of Ebola, Marburg, Junin, Lassa, and HIV-1 virus like particles (VLPs) and transmission of live Influenza, Junin, Lassa, Ebola, Marburg and Vesicular Stomatits (VSV) viruses in culture. The disclosure of the present invention demonstrates a novel role for Orai1-mediated calcium signals in late steps of viral particle trafficking or budding from cells, such as in late steps of Ebola, Marburg, Lassa, Junin, HIV-1-mediated VLP formation, and in the production of live Influenza A, Junin, Lassa, Ebola, Marburg and Vesicular Stomatits Virus (VSV).

Further, Ca²⁺ plays a role in the function, assembly, or activation of several host ESCRT proteins required for efficient budding, including ALG-2 interaction protein X (Alix) and its binding partner, α-1,3/1,6-mannosyltransferase (ALG-2). Alix interacts through a novel VP40 L domain and can rescue VLP formation in the absence of Nedd4 and Tsg101 interacting L-domain via a Ca²⁺-dependent mechanism. Alix/ALG-2 interactions normally control endosomal sorting and membrane repair by activating a Ca²⁺-permeant channel Transient Receptor Potential Mucolipin I (TRPML1), which normally controls endosomal sorting and regulates membrane repair. At the plasma membrane, TRPML1 can produce plasma membrane tubulovesicular structures (TVS), which are indistinguishable from those generated by VP40. As demonstrated herein, suppression of either Alix or TRPML1 inhibits VP40-mediated VLP formation. In certain embodiments, VP40 and Z proteins trigger intracellular Ca²⁺ signaling by activating Orai1, thereby orchestrating Ca²⁺-dependent ESCRT protein activation and interactions (e.g., Alix and ALG-2) or signals that recruit TRPML1 to the plasma membrane to facilitate early (protrusion) and late (scission) stages of filovirus budding.

The present invention further relates in part to the unexpected discovery that CRAC channel inhibitors and/or TRPML1 channel activation inhibitors prevent or impair viral budding of human and animal enveloped RNA pathogens in a subject, and thus may be effectively used as antiviral agents in a subject.

In certain embodiments, the compounds useful within the invention treat a viral infection in a subject. In other embodiments, the viral infection is caused by at least one single strand RNA virus. In yet other embodiments, the infection is caused by at least one virus selected from the group consisting of a filovirus (such as, but not limited to, Ebola or Marburg virus), arenavirus (such as, but not limited to, Juinin or Lassa fever virus), rhabdovirus (such as, but not limited to, rabies, vesicular stomatitis, or emerging lyssavirus), paramyxovirus (such as, but not limited to, Nipah or Hendra virus), retrovirus (such as, but not limited to, HIV-1, HIV-2, or human T-cell leukemia virus, also known as HTLV-1), orthomyxovirus (such as, but not limited to, Influenza A or Influenza B) and any combinations thereof. In other embodiments, the subject is a mammal.

### DEFINITIONS

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, virology and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

In one aspect, the terms “co-administered” and “co-administration” as relating to a subject refer to administering to the subject a compound of the invention or salt thereof along with a compound that may also treat a disease or disorder contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, the term “composition” or “pharmaceutical composition” refers to a mixture of at least one compound useful within the invention with a pharmaceutically acceptable carrier. The pharmaceutical composition facilitates administration of the compound to a subject.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “EBOV’ refers to Ebola viruses.

As used herein, an “effective amount” or “therapeutically effective amount” or “pharmaceutically effective amount” of a compound is that amount of compound that is sufficient to provide a beneficial effect to the subject to which the compound is administered.

As used herein, the term “FC2122” refers to 3-(4-methyl-1,5-diphenyl-1H-pyrazol-3-yl)-2-phenylpropanoic acid, or a salt or solvate thereof.

“Instructional material” as that term is used herein includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “JUNV” refers to Junin viruses.

As used herein, the term “LFV” refers to Lassa fever viruses.

As used herein, the term “MARV” refers to Marburg viruses.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

The term “prevent” or “preventing” or “prevention” as used herein means avoiding or delaying the onset of symptoms associated with a disease or condition in a subject that has not developed such symptoms at the time the administering of an agent or compound commences. Disease, condition and disorder are used interchangeably herein.

As used herein, the term “RABV” refers to rabies viruses.

By the term “specifically bind” or “specifically binds” as used herein is meant that a first molecule preferentially binds to a second molecule (e.g., a particular receptor or enzyme), but does not necessarily bind only to that second molecule.

As used herein, a “subject” may be a human or non-human mammal or a bird. Non-human mammals include, for example, livestock and pets, such as ovine, bovine, porcine, canine, feline and murine mammals. Preferably, the subject is human.

As used herein, the term “thapsigargin” or “Tg” refers to (3S,3aR,4S,6S,6aR,7S,8S,9bS)-6-(acetyloxy)-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-yl octanoate or a solvate or adduct thereof.

The term “treat” or “treating” or “treatment” as used herein means reducing the frequency or severity with which symptoms of a disease or condition are experienced by a subject by virtue of administering an agent or compound to the subject.

As used herein, the term “VLPs” refers to virus-like particles.

As used herein, the term “VSV” refers to vesicular stomatitis viruses.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkenyl” employed alone or in combination with other terms means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—CCH. The term “homopropargylic” refers to a group exemplified by —CH₂CH₂—CCH. The term “substituted propargylic” refers to a group exemplified by —CR₂—CCR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—CCR, wherein each occurrence of R is independently H, alkyl, substituted alkyl, alkenyl or substituted alkenyl, with the proviso that at least one R group is not hydrogen.

As used herein, the term “aromatic” refers to a carbocycle or heterocycle with one or more polyunsaturated rings and having aromatic character, i.e. having (4n+2) delocalized it (pi) electrons, where n is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “arylalkyl” means a functional group wherein an alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroarylalkyl” means a functional group wherein an alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “cycloalkyl” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “halo” or “halogen” employed alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

As used herein, the term “substituted alkyl” or “substituted cycloalkyl” or “substituted alkenyl” or “substituted alkynyl” means alkyl, cycloalkyl, alkenyl or alkynyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

### DISCLOSURE

The present invention relates in part to the identification of potent, broad-spectrum antiviral compounds. In certain embodiments, the compounds of the invention target high-priority pathogens for which no approved treatments are available.

As described herein, compounds that inhibit calcium release activated calcium (CRAC) channels can be used to prevent viral budding, spread and transmission. As such, CRAC channel inhibitors are useful within the methods of the invention.

Non-limiting examples of CRAC channel inhibitors, or salts or solvate thereof, that are useful within the methods of the invention include:

lanthanides, such as La³⁺ and Gd³⁺;

N-(4-(3,5-bis(trifluoromethyl)-1H-pyrazol-1-yl)phenyl)-4-methyl-1,2,3-thiadiazole-5-carboxamide (Pyr2/BTP2/YM58483) (Ishikawa, et al., 2003, J. Immunol. 170(9):4441-4449; Takezawa, et al., 2006, Mol. Pharm. 69(4):1413-1420; Zitt, et al., 2004, J. Biol. Chem. 279(13):12427-12437)

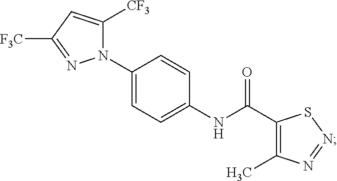 [SMILES: Cc1nnsc1C(=O)Nc1ccc(-n2nc(C(F)(F)F)cc2C(F)(F)F)cc1]

ethyl 1-(4-(2,3,3-trichloroacrylamido)phenyl)-5-(trifluoromethyl)-1H-pyrazole-4-carboxylate (Pyr3)

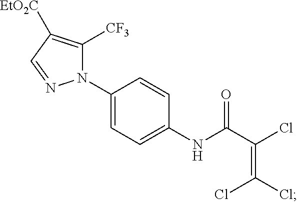 [SMILES: CCOC(=O)c1cnn(-c2ccc(NC(=O)C(Cl)=C(Cl)Cl)cc2)c1C(F)(F)F]

N-(4-(3,5-bis(trifluoromethyl)-1H-pyrazol-1-yl)phenyl)-3-fluoroisonicotinamide (Pyr6)

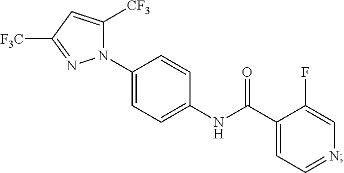 [SMILES: O=C(Nc1ccc(-n2nc(C(F)(F)F)cc2C(F)(F)F)cc1)c1ccncc1F]

N-(4-(3,5-bis(trifluoromethyl)-1H-pyrazol-1-yl)phenyl)-4-methylbenzenesulfonamide (Pyr10)

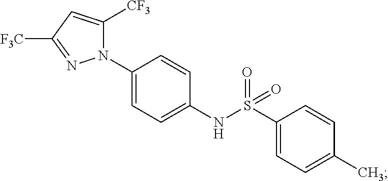 [SMILES: Cc1ccc(S(=O)(=O)Nc2ccc(-n3nc(C(F)(F)F)cc3C(F)(F)F)cc2)cc1]

2-aminoethoxydiphenylborate (2-APB)

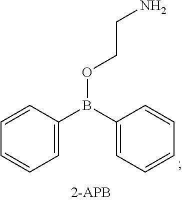 [SMILES: NCCOB(c1ccccc1)c1ccccc1]

2,2′-((((oxybis(methylene))bis(3,1-phenylene))bis(phenylboranediyl))bis(oxy)) bis(ethan-1-amine) (DPB162-AE);

2,2′-((((oxybis(methylene))bis(4,1-phenylene))bis(phenylboranediyl))bis(oxy)) bis(ethan-1-amine) (DPB163-AE)(Goto, et al., 2010, Cell Calcium 47:1-10);

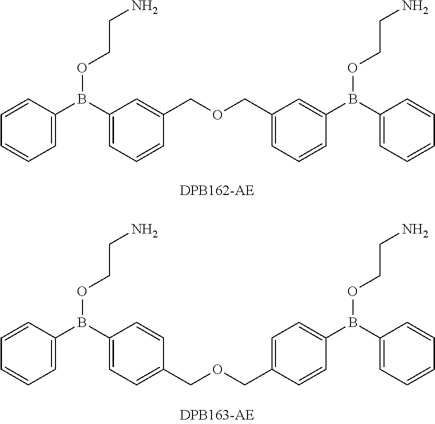 [SMILES: NCCOB(c1ccccc1)c1cccc(COCc2cccc(B(OCCN)c3ccccc3)c2)c1 ; NCCOB(c1ccccc1)c1ccc(COCc2ccc(B(OCCN)c3ccccc3)cc2)cc1]

capsaicin (8-methyl-N-vanillyl-(trans)-6-nonenamide; Fischer, et al., 2001, J. Pharm. Exp. Ther. 299(1):238-246);

NPPB (5-nitro-2-(3-phenylpropylamino)-benzoic acid) (Gericke, et al., 1994, Eur. J. Pharm. 269(3):381-384; Li, et al., 2000, Eur. J. Pharm. 394(2-3):171-179; Reinsprecht, et al., 1995, Mol. Pharm. 47(5):1014-1020)

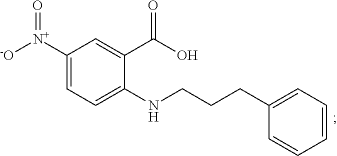

DES (diethylstilbestrol) (Zakharov, et al., 2004, Mol. Pharm. 66(3):702-707)

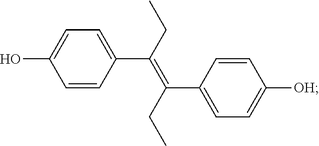 [SMILES: CC/C(=C(/CC)c1ccc(O)cc1)c1ccc(O)cc1]

BEL (bromenol lactone, or E-6-(bromoethylene)tetrahydro-3-(1-naphthyl)-2H-pyran-2-one) (Winstead, et al., 2000, Biochim. Biophys. Acta 1488(1-2):28-39);

CM2489 and CM3457 (CalciMedica, La Jolla, Calif.); cholestatic bile acids (such as taurolithocholic acid (TLCA; 2-[4-[(3R,5R,8R,9S,10S,13R,14S,17R)-3-hydroxy-10,13-dimethyl-2,3,4,5,6,7,8,9,11,12,14,15,16,17-tetradecahydro-1H-cyclopenta[a]phenanthren-17-yl]pentanoylamino]ethanesulfonic acid), lithocholic acid (LCA; (4R)-4-[(3R,5R,8R,9S,10S,13R,14S,17R)-3-Hydroxy-10,13-dimethyl-2,3,4,5,6,7,8,9,11,12,14,15,16,17-tetradecahydro-1H-cyclopenta[a]phenanthren-17-yl]pentanoic acid), cholic acid (CA; (R)-4-((3R,5S,7R,8R,9S,10S,12S,13R,14S,17R)-3,7,12-trihydroxy-10,13-dimethylhexadecahydro-1H-cyclopenta[a]phenanthren-17-yl)pentanoic acid) and taurocholic acid (TCA; 2-{[(3α,5β,7α,12α)-3,7,12-trihydroxy-24-oxocholan-24-yl]amino}ethanesulfonic acid)); Aromataris, et al., 2008, Biochim. Biophys. Acta 1783(5):874-885);

ML-9 (1-(5-chloronaphthalene-1-sulfonyl)homopiperazine) (Tran, et al., 2001, Arter. Thromb. Vasc. Biol. 21(4):509-515)

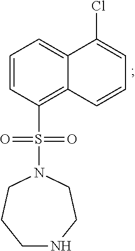 [SMILES: O=S(=O)(c1cccc2c(Cl)cccc12)N1CCCNCC1]

R02959 (2,6-difluoro-N-{5-[4-methyl-1-(5-methyl-thiazol-2-yl)-1,2,5,6-tetrahydro-pyridin-3-yl]-pyrazin-2-yl}-benzamide)

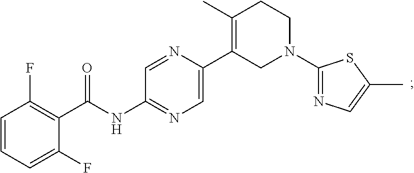 [SMILES: CC1=C(c2cnc(NC(=O)c3c(F)cccc3F)cn2)CN(c2ncc(C)s2)CC1]

the compounds disclosed in PCT Patent Application Nos. WO2005/009954, WO2005009539 and WO2010039236, the entire disclosures of which are incorporated herein by reference in their entireties, including Synta-66 (N-(2′,5′-dimethoxy[1,1′-biphenyl]-4-yl)-3-fluoro-4-pyridinecarboxamide)

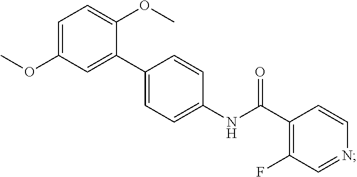 [SMILES: COc1ccc(OC)c(-c2ccc(NC(=O)c3ccncc3F)cc2)c1]

the compounds disclosed in PCT Patent Application No. WO2010/122089, which is incorporated herein by reference in its entirety, including: GSK-5503A (2,6-difluoro-N-(1-(2-phenoxybenzyl)-1H-pyrazol-3-yl)benzamide)

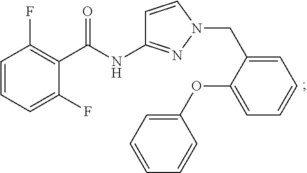 [SMILES: O=C(Nc1ccn(Cc2ccccc2Oc2ccccc2)n1)c1c(F)cccc1F]

and
GSK-7975A (2,6-difluoro-N-(1-(4-hydroxy-2-(trifluoromethyl)benzyl)-1H-pyrazol-3-yl)benzamide)

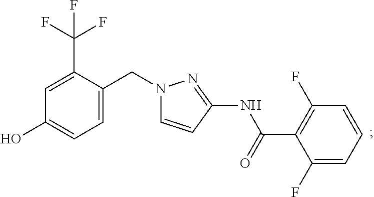 [SMILES: O=C(Nc1ccn(Cc2ccc(O)cc2C(F)(F)F)n1)c1c(F)cccc1F]

FCC2121 (4-[3-(diphenylmethyl)-1,2,4-oxadiazol-5-yl]piperidineyl]piperidine)

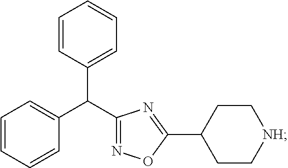 [SMILES: c1ccc(C(c2ccccc2)c2noc(C3CCNCC3)n2)cc1]

FCC2122 (3-(4-methyl-1,5-diphenyl-1H-pyrazol-3-yl)-2-phenylpropanoic acid)

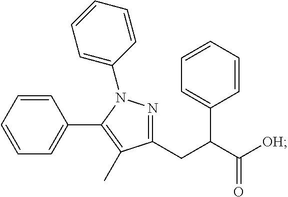 [SMILES: Cc1c(CC(C(=O)O)c2ccccc2)nn(-c2ccccc2)c1-c1ccccc1]

FC-2399 (2-(4-Chloro-phenyl)-3-[1-(4-chloro-phenyl)-5-methyl-1H-pyrazol-3-yl]-propionic acid)

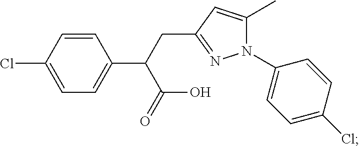 [SMILES: Cc1cc(CC(C(=O)O)c2ccc(Cl)cc2)nn1-c1ccc(Cl)cc1]

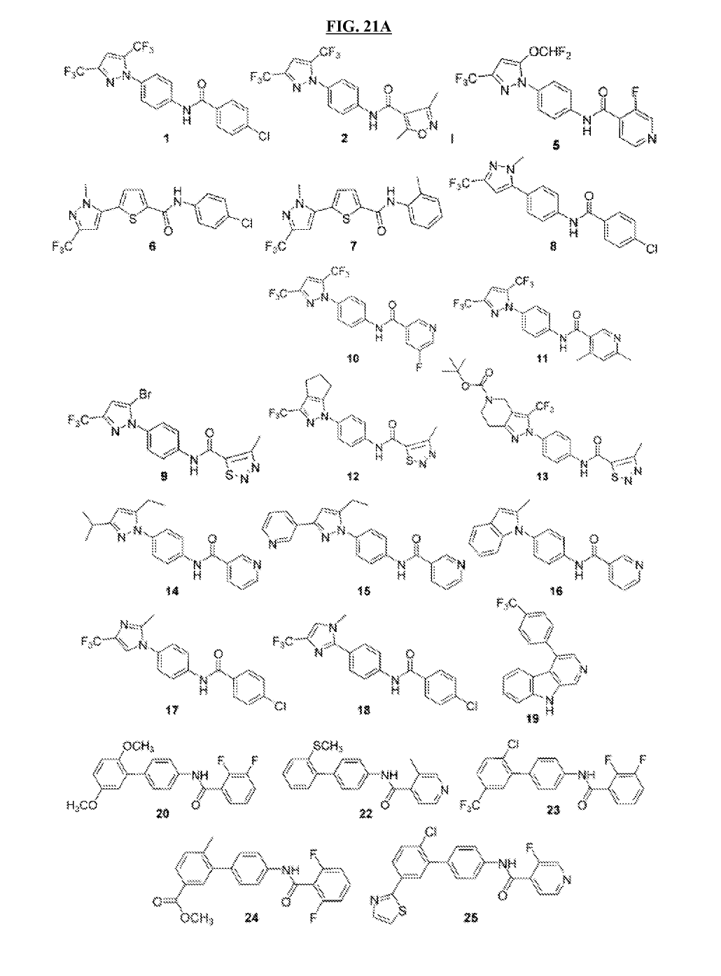

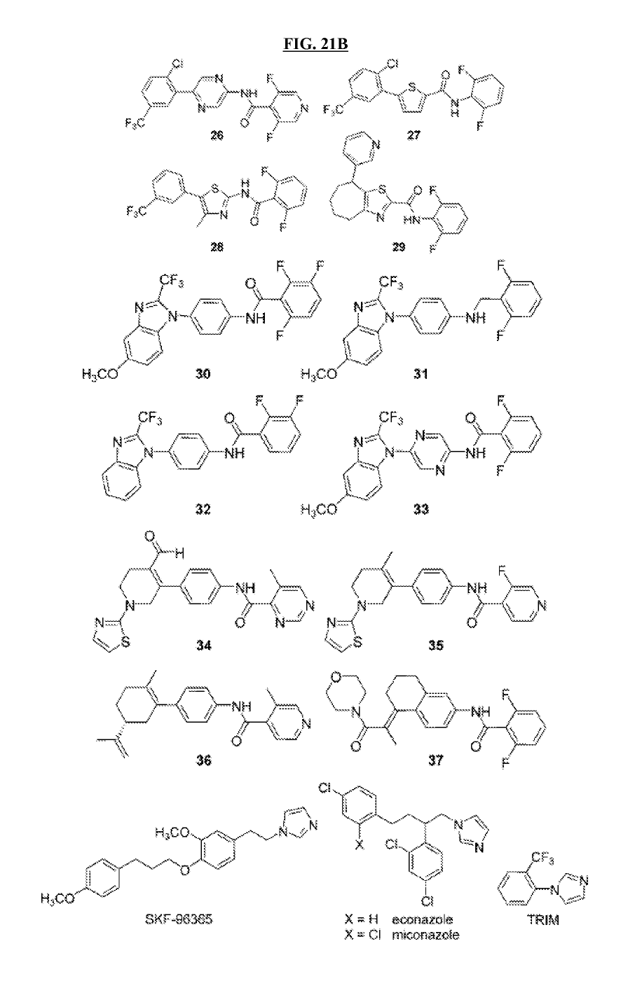

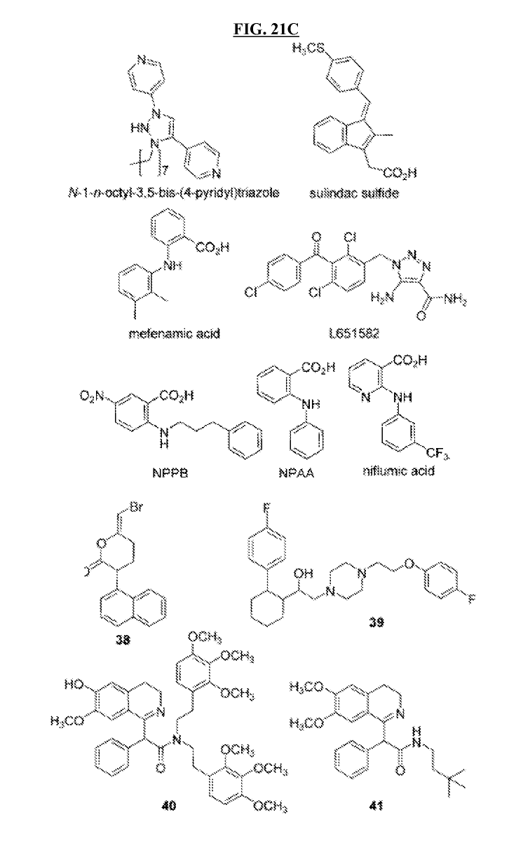

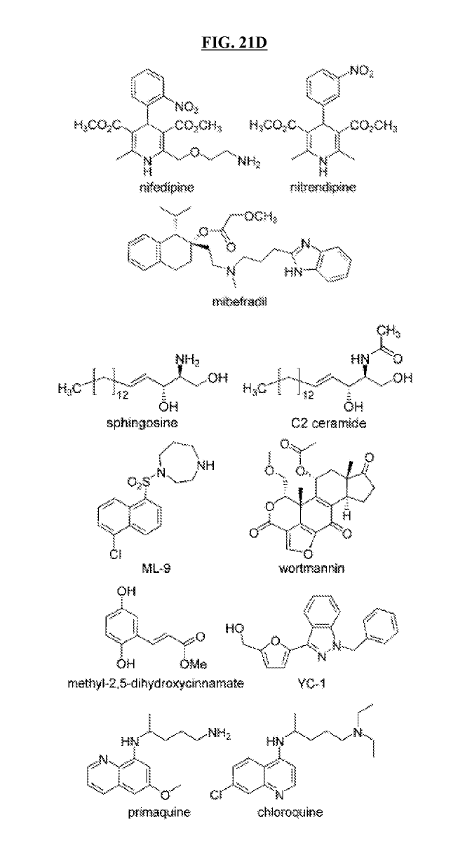

the compounds disclosed in Sweeney, et al., 2009, ChemMedChem 4:706-718, which entire disclosure is incorporated herein by reference in its entirety, such as but not limited to the compounds illustrated in FIGS. 21A-21D;

the compounds disclosed in PCT Patent Application Publication No. WO 2010/122089, which entire disclosure is incorporated herein by reference in its entirety, including: N-[1-({2-Chloro-5-[(cyclopropylmethyl)oxy]phenyl}methyl)-1H-pyrazol-3-yl]-2,6-difluorobenzamide; N-{1-[(2,4-Dichlorophenyl)methyl]-1H-pyrazol-3-yl}-2,6-difluorobenzamide; 2-Bromo-N-{1-[(2,4-dichlorophenyl)methyl]-1H-pyrazol-3-yl}-6-fluorobenzamide; 2-Chloro-N-{1-[(2,4-dichlorophenyl)methyl]-1H-pyrazol-3-yl}-6-fluorobenzamide; 2,6-Dichloro-N-{1-[(2,4-dichlorophenyl)methyl]-1H-pyrazol-3-yl}benzamide; N-{1-[(2,4-dichlorophenyl)methyl]-1H-pyrazol-3-yl}-3,5-difluoro-4-pyridinecarboxamide; N-[1-({5-chloro-2-[(phenylmethyl)oxy]phenyl}methyl)-1H-pyrazol-3-yl]-2,6-25 difluorobenzamide; N-{1-[(2,6-dichlorophenyl)methyl]-1H-pyrazol-3-yl}-2,6-difluorobenzamide; N-[1-({5-chloro-2-[(2-methylpropyl)oxy]phenyl}methyl)-1H-pyrazol-3-yl]-2,6-difluorobenzamide; N-(1-{[2-bromo-5-(methyloxy)phenyl]methyl}-1H-pyrazol-3-yl)-2,6-difluorobenzamide; N-(1-{[5-chloro-2-(methyloxy)phenyl]methyl}-1H-pyrazol-3-yl)-2,6-difluorobenzamide; 2,6-Difluoro-N-(1-{[2-(phenyloxy)phenyl]methyl}-1H-pyrazol-3-yl)benzamide; N-[1-({5-bromo-2-[(phenylmethyl)oxy]phenyl}methyl)-1H-pyrazol-3-yl]-2,6-difluorobenzamide; 2,6-Difluoro-N-[1-({2-[(trifluoromethyl)oxy]phenyl}methyl)-1H-pyrazol-3-yl]benzamide; 2,6-Difluoro-N-(1-{[4-[(phenylmethyl)oxy]-2-(trifluoromethyl)phenyl]methyl}-1H-pyrazol-3-yl)benzamide; N-{1-[(2-Bromo-6-chlorophenyl)methyl]-1H-pyrazol-3-yl}-2,6-difluorobenzamide; 2,6-Difluoro-/V-[1-({2-[(phenylmethyl)oxy]phenyl}methyl)-1H-pyrazol-3-yl]benzamide; N/-[1-({2-chloro-5-[(2-methylpropyl)oxy]phenyl}methyl)-1H-pyrazol-3-yl]-2,6-difluorobenzamide; N-(1-{[4-[(cyclopropylmethyl)oxy]-2-(trifluoromethyl)phenyl]methyl}-1H-pyrazol-3-yl)-2,6-difluorobenzamide; 2,6-Difluoro-N-(1-{[4-iodo-2-(trifluoromethyl)phenyl]methyl}-1H-pyrazol-3-yl)benzamide; 2,6-Difluoro-N-(1-{[4-methyl-2-(trifluoromethyl)phenyl]methyl}-1H-pyrazol-3-yl)benzamide; N-(1-{[4-cyclopropyl-2-(trifluoromethyl)phenyl]methyl}-1H-pyrazol-3-yl)-2,6-difluorobenzamide; 2,6-Difluoro-N-{1-[(4-iodo-2-methylphenyl)methyl]-1H-pyrazol-3-yl}benzamide; N-(1-{[4-chloro-2-(trifluoromethyl)phenyl]methyl}-1H-pyrazol-3-yl)-2,6-difluorobenzamide; 2-Fluoro-N-(1-{[4-iodo-2-(trifluoromethyl)phenyl] methyl}-1H-pyrazol-3-yl)benzamide; 2-Chloro-N-(1-{[4-cyclopropyl-2-(trifluoromethyl) phenyl]methyl]-1H-pyrazol-3-yl)benzamide; N-(1-{[4-cyclopropyl-2-(trifluoromethyl) phenyl]methyl}-1H-pyrazol-3-yl)-2-fluorobenzamide; 2,6-Difluoro-N-(1-{[5-iodo-2-(trifluoromethyl)phenyl]methyl}-1H-pyrazol-3-yl)benzamide; 2,6-Difluoro-N-(1-{[2-fluoro-6-(trifluoromethyl)phenyl]methyl}-1H-pyrazol-3-yl)benzamide; 2,6-Difluoro-N-(1-{[4-hydroxy-2-(trifluoromethyl)phenyl]methyl}-1H-pyrazol-3-yl)benzamide;

a compound of formula (III):

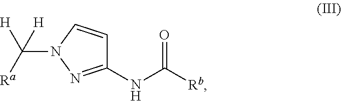

wherein

R^{a} is a group of formula (a1)

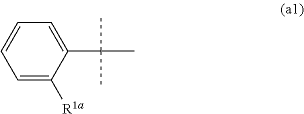

in which R^{1a} is C₁₋₆alkyl, CF₃, OCF₃, C₁₋₆alkoxy or R^{1a} is a group L⁴-Z¹ in which L⁴ is O, CH₂, OCH₂ or CH₂O and Z¹ is C₃₋₇ cycloalkyl or aryl; or

R^{a} is a group of formula (a2)

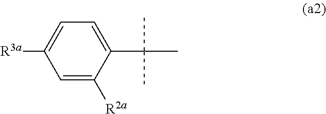

wherein R^{2a} is halogen, C₁₋₆alkyl, CF₃ or OCH₂Ph; and R^{1a} is halogen, C₁₋₆alkyl, C₁₋₆alkoxy, hydroxy, C₃₋₇cycloalkyl, CO₂C₁₋₄alkyl or R^{1a} is a group L⁵-Z² in which L⁵ is O, CH₂ or O(CH₂), wherein n is an integer from 1-7; and Z² is hydroxy, methoxy, CO₂C₁₋₄alkyl, C₃₋₇cycloalkyl, aryl or heteroaryl; or

R^{a} is a group of formula (a3)

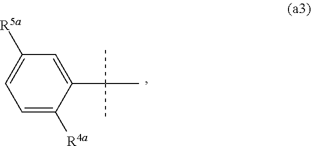

wherein R^{4a} is halogen, C₁₋₆alkyl, C₁₋₆alkoxy, CF₃ or OCH₂Ph; and R^{5a} is halogen, C₁₋₆alkyl, hydroxy, C₁-6alkoxy optionally substituted by methoxy, or R^{5a} is a group L³-Z³ in which L³ is a single bond, O, CH₂, OCH₂ or CH₂O and Z³ is C₃₋₇cycloalkyl, aryl or heteroaryl; or

R^{a} is a group of formula (a4)

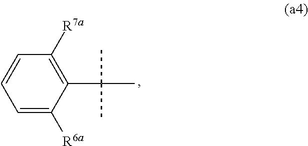

wherein R^{6a} is Cl, Br, C₁₋₆ alkyl or CF₃; and R^{7a} is halogen, C₁₋₆ alkyl, CF₃, OCF₃, OCHF₂, C₁₋₆alkoxy or R^{7a} is a group L⁴-Z⁴ in which L⁴ is OCH₂ and Z⁴ is C₃₋₇ cycloalkyl;

R^{b} is a group of formula (b)

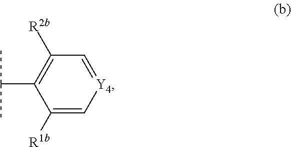

wherein Y₄ is CH or N; R^{1b} is halogen, C₁₋₆alkyl or CF₃; R^{2b} is H, halogen, C₁₋₆alkyl or C₁₋₆alkoxy or a salt thereof;

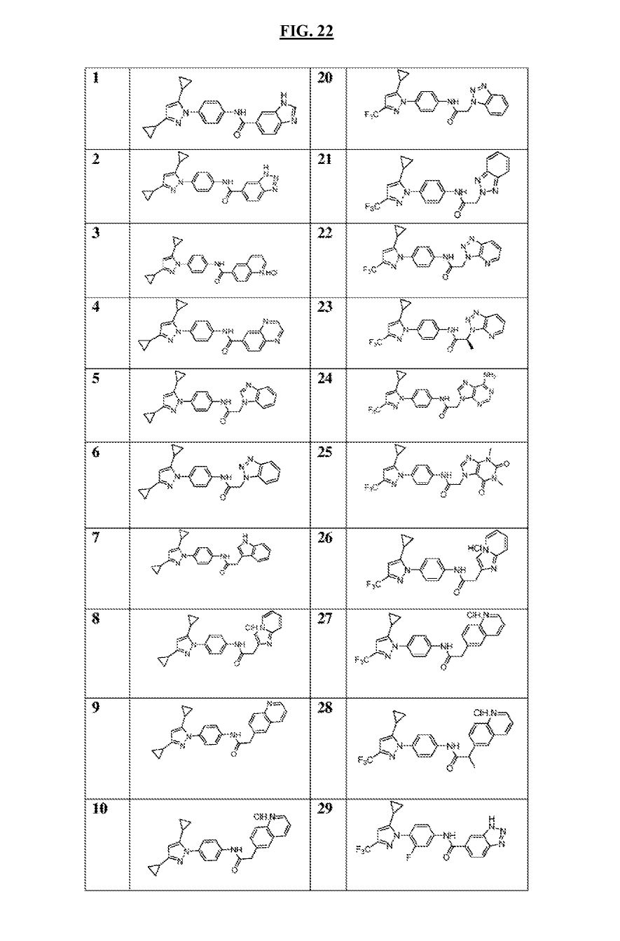

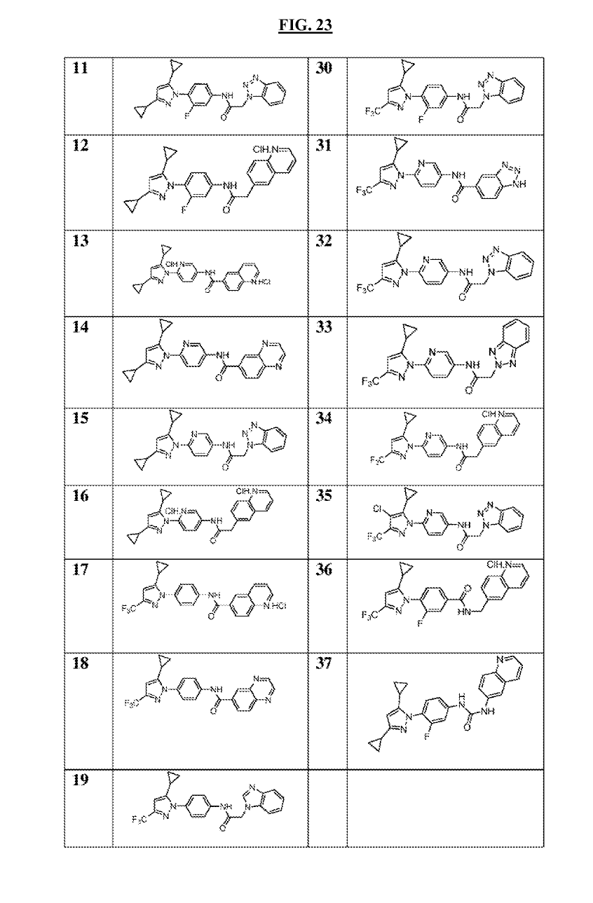

the compounds disclosed in U.S. Patent Application Publication No. US2011/0112058 (illustrated in FIGS. 22 and 23), which entire disclosure is incorporated herein by reference in its entirety, including: N-[4-(3,5-dicyclopropyl-1H-pyrazol-1-yl)phenyl]-1H-benzo[d]imidazole-6-carboxamide; N-[4-(3,5-dicyclopropyl-1H-pyrazol-1-yl)phenyl]-1H-benzo[d][1,2,3]triazole-6-carboxamide; N-[4-(3,5-dicyclopropyl-1H-pyrazol-1-yl)phenyl]quinoline-6-carboxamide; N-[4-(3,5-dicyclopropyl-1H-pyrazol-1-yl)phenyl] quinoxaline-6-carboxamide; 2-(1H-benzo[d]imidazol-1-yl)-N-[4-(3,5-dicyclopropyl-1H-pyrazol-1-yl)phenyl]acetamide; 2-(1H-benzo[d][1,2,3] triazol-1-yl)-N-[4-(3,5-dicyclopropyl-1H-pyrazol-1-yl)phenyl] acetamide; N-[4-(3,5-dicyclopropyl-1H-pyrazol-1-yl)phenyl]-2-(1H-indol-3-yl)acetamide; N-[4-(3,5-dicyclopropyl-1H-pyrazol-1-yl)phenyl]-2-(imidazo[1,2-a]pyridin-2-yl) acetamide; N-[4-(3,5-dicyclopropyl-1H-pyrazol-1-yl)phenyl]-2-(quinolin-6-yl)acetamide; N-[4-(3,5-dicyclopropyl-1H-pyrazol-1-yl)phenyl]-2-(quinolin-6-yl)acetamide; 2-(1H-benzo[d][1,2,3]triazol-1-yl)-N-(4-(3,5-dicyclopropyl-1H-pyrazol-1-yl)-3-fluorophenyl) acetamide; N-[4-(3,5-dicyclopropyl-1H-pyrazol-1-yl)-3-fluorophenyl]-2-(quinolin-6-yl) acetamide; N-[6-(3,5-dicyclopropyl-1H-pyrazol-1-yl)pyridin-3-yl]quinoline-6-carboxamide; N-[6-(3,5-dicyclopropyl-1H-pyrazol-1-yl)pyridin-3-yl]quinoxaline-6-carboxamide; 2-(1H-benzo[d][1,2,3]triazol-1-yl)-N-[6-(3,5-dicyclopropyl-1H-pyrazol-1-yl)pyridin-3-yl] acetamide; N-[6-(3,5-dicyclopropyl-1H-pyrazol-1-yl)pyridin-3-yl]-2-(quinolin-6-yl) acetamide; N-{4-[5-cyclopropyl-3-(trifluoromethyl)-1H-pyrazol-1-yl]phenyl}quinoline-6-carboxamide; N-{4-[5-cyclopropyl-3-(trifluoromethyl)-1H-pyrazol-1-yl]phenyl}quinoxaline-6-carboxamide; 2-(1H-benzo[d]imidazol-1-yl)-N-{4-[5-cyclopropyl-3-(trifluoromethyl)-1H-pyrazol-1-yl] phenyl]acetamide; 2-(1H-benzo[d][1,2,3]triazol-1-yl)-N-{4-[5-cyclopropyl-3-(trifluoromethyl)-1H-pyrazol-1-yl]phenyl}acetamide; 2-(2H-benzo[d][1,2,3]triazol-2-yl)-N-{4-[5-cyclopropyl-3-(trifluoromethyl)-1H-pyrazol-1-yl]phenyl}acetamide; 2-(3H-[1,2,3]triazolo[4,5-b]pyridin-3-yl)-N-{4-[5-cyclopropyl-3-(trifluoromethyl)-1H-pyrazol-1-yl] phenyl}acetamide; (S)-2-(3H-[1,2,3]triazolo[4,5-b]pyridin-3-y])-N-{4-[5-cyclopropyl-3-(trifluoromethyl)-1H-pyrazol-1-yl]phenyl}propanamide; 2-(6-amino-9H-purin-9-yl)-N-{4-[5-cyclopropyl-3-(trifluoromethyl)-1H-pyrazol-1-yl]phenyl}acetamide; N-(4-(5-cyclopropyl-3-(trifluoromethyl)-1H-pyrazol-1-yl)phenyl)-2-(1,3-dimethyl-2,6-dioxo-2,3-dihydro-1H-purin-7(6H)-yl)acetamide; N-{4-[5-cyclopropyl-3-(trifluoromethyl)-1H-pyrazol-1-yl) phenyl)-2-(imidazo[1,2-a] pyridin-2-yl) acetamide; N-{4-[5-cyclopropyl-3-(trifluoromethyl)-1H-pyrazol-1-yl]phenyl}-2-(quinolin-6-yl)acetamide; N-{4-[5-cyclopropyl-3-(trifluoromethyl)-1H-pyrazol-1-yl]phenyl}-2-(quinolin-6-yl)propanamide; N-{4-[5-cyclopropyl-3-(trifluoromethyl)-1H-pyrazol-1-yl]-3-fluorophenyl}-1H-benzo[d] [1,2,3]triazole-6-carboxamide; 2-(1H-benzo[d][1,2,3]triazol-1-yl)-N-{4-[5-cyclopropyl-3-(trifluoromethyl)-1H-pyrazol-1-yl]-3-fluorophenyl}acetamide; N-{6-[5-cyclopropyl-3-(trifluoromethyl)-1H-pyrazol-1-yl]pyridin-3-yl}-1H-benzo[d][1,2,3] triazole-5-carboxamide; 2-(1H-benzo[d][1,2,3]triazol-1-yl)-N-{6-[5-cyclopropyl-3-(trifluoromethyl)-1H-pyrazol-1-yl]pyridin-3-yl}acetamide; 2-(2H-benzo[d][1,2,3]triazol-2-yl)-N-{6-[5-cyclopropyl-3-(trifluoromethyl)-1H-pyrazol-1-yl]pyridin-3-yl}acetamide; N-{6-[5-cyclopropyl-3-(trifluoromethyl)-1H-pyrazol-1-yl]pyridin-3-yl}-2-(quinolin-6-yl)acetamide; 2-(1H-benzo[d][1,2,3]triazol-1-yl)-N-{6-[4-chloro-5-cyclopropyl-3-(trifluoromethyl)-1H-pyrazol-1-yl]pyridin-3-yl}acetamide; 4-[5-cyclopropyl-3-(trifluoromethyl)-1H-pyrazol-1-yl]-3-fluoro-N-(quinolin-6-ylmethyl)benzamide; 1-[4-(3,5-dicyclopropyl-1H-pyrazol-1-yl)phenyl]-3-(quinolin-6-yl)urea;

a compound of formula (IV):

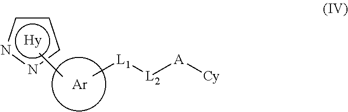

or a tautomer thereof, prodrug thereof, N-oxide thereof, pharmaceutically acceptable ester thereof or pharmaceutically acceptable salt thereof, wherein Ring Hy represents one of the structures in formula (V);

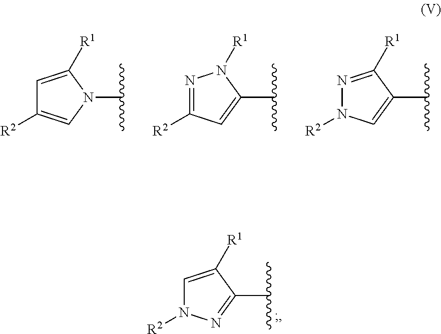

wherein Ring Hy is optionally substituted with R′″; R¹ and R² are the same or different and are independently selected from CH₃, CH₂F, CHF₂, CF₃, substituted or unsubstituted C₍₃₋₅₎ cycloalkyl, CH₂—OR^{e}, CH₂—NR^{e}R^{f}, CN and COOH with the proviso that: a) both R¹ and R² at the same time do not represent CF₃, b) both R¹ and R² at the same time do not represent CH₃, c) when R¹ is CF₃ then R² is not CH₃, and d) when R¹ is CH₃ then R² is not CF₃; ring Ar represents:

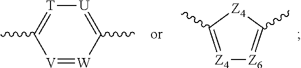

wherein T, U, V and W are each independently selected from CR^{e} and N; Z⁴, Z⁵ and Z⁶ are the same or different and are independently selected from CRₑ, CR^{e}R^{f}, O, S and —NR^{e}, with the proviso that at least one of Z⁴, Z⁵ and Z⁶ represents O, S or —NR^{e}; L₁ and L₂ together represent —NH—C(═X)—, —NH—S(═O)q-, —C(═X)NH—, —NH—CR′R″ or —S(═O)_{q}NH—; A is absent or selected from —(CR′R″)—, O, S(═O)_{q}, C(═X) and —NR^{e}; each occurrence of R′ and R″ are the same or different and are independently selected from hydrogen, hydroxy, cyano, halogen, —OR^{e}, —COOR^{e}, —S(═O)_{q}—R^{e}, —NR^{e}R^{f}, —C(═X)—R^{e}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl, or R′ and R″ directly bound to a common atom, may be joined to form a substituted or unsubstituted saturated or unsaturated 3-6 member ring, which may optionally include one or more heteroatoms which may be same or different and are selected from O, NR^{e} and S; R″ is selected from hydrogen, hydroxy, cyano, halogen, —OR^{e}, —COOR^{e}, —S(═O)_{q}—R^{e}, —NR^{e}R^{f}, —C(═X)—R^{e}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl; each occurrence of X is independently selected from O, S and —NR^{e}; Cy is a bicyclic ring selected from substituted or unsubstituted cycloalkyl group, substituted or unsubstituted heterocyclyl, substituted or unsubstituted aryl, and substituted or unsubstituted heteroaryl; each occurrence of R^{e} and R^{f} are the same or different and are independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NR^{c}R^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{e} and R^{f} are directly bound to the same atom, they may be joined to form a substituted or unsubstituted saturated or unsaturated 3-10 membered ring, which may optionally include one or more heteroatoms which may be same or different and are selected from O, NR^{c} and S; each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 membered ring, which may optionally include one or more heteroatoms which are the same or different and are selected from O, NH and S; each occurrence of Y is selected from O, S and —NR^{e}; and each occurrence of q independently represents an integer 0, 1 or 2;

According to one preferred embodiment, Hy is selected from one of the structures in formula (VI);

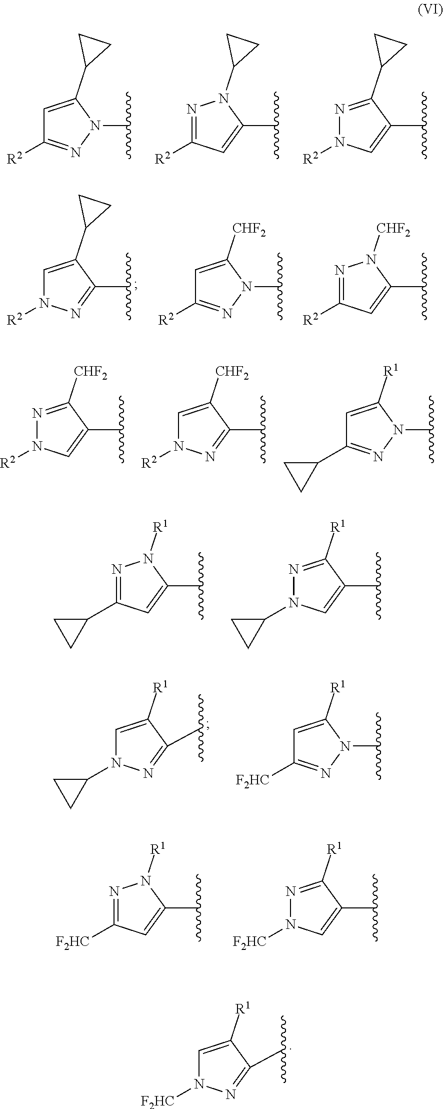

In certain embodiments, in the compound of formula (IV) Hy is selected from one of the structures in formula (VII);

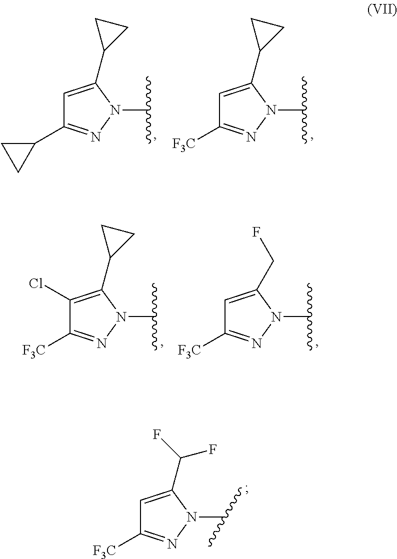 [SMILES: *n1nc(C2CC2)cc1C1CC1 ; *n1nc(C(F)(F)F)cc1C1CC1 ; *n1nc(C(F)(F)F)c(Cl)c1C1CC1 ; *n1nc(C(F)(F)F)cc1CF ; *n1nc(C(F)(F)F)cc1C(F)F]

Yet another embodiment is a compound having the formula (IA):

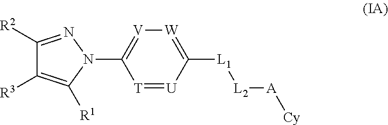

Yet another embodiment is a compound having the formula (IA-1);

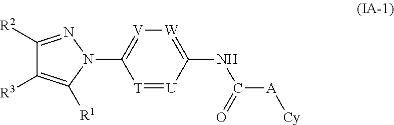

the compounds disclosed in U.S. Patent Application Publication No. US2013/0158040, which entire disclosure is incorporated herein by reference in its entirety, including: 4-[6-(2-chloro-6-fluoro-phenyl)-5H-pyrrolo[3,2-d]pyrimidin-2-yl]-3,N,N-trimethyl-benzenesulfonamide; 6-(2-Chloro-phenyl)-2-(2-methyl-5-trifluoromethyl-2H-pyrazol-3-yl)-5H-pyrrolo[2,3-b]pyrazine; 4-[6-(2-Chloro-phenyl)-5H-pyrrolo[2,3-b]pyrazin-2-yl]-3-methyl-benzoic acid methyl ester; 4-(6-(2-Chlorophenyl)-5H-pyrrolo[2,3-b]pyrazin-2-yl)-N,N,3-trimethylbenzenesulfonamide; 6-(2-chloro-6-fluorophenyl)-2-(1-methyl-3-(trifluoromethyl)-1H-pyrazol-5-yl)-5H-pyrrolo[2,3-b]pyrazine; 6-Cyclohexyl-2-(1-methyl-3-(trifluoromethyl)-1H-pyrazol-5-yl)-5H-pyrrolo[2,3-b]pyrazine; 4-(6-Cyclohexyl-5H-pyrrolo[2,3-b]pyrazin-2-yl)-N,N,3-trimethylbenzenesulfonamide;

a compound of formula (II):

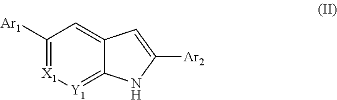

wherein: one of X₁ and Y₁ is C and the other is N; Ar₁ is unsubstituted cycloalkyl, unsubstituted phenyl or phenyl mono- or bi-substituted independently with halogen; Ar₂ is phenyl, unsubstituted or mono- or bi-substituted independently with lower alkyl or haloalkyl; or a pharmaceutically acceptable salt thereof;

the compounds disclosed in PCT Patent Application Publication No. WO 2013/164773, which entire disclosure is incorporated herein by reference in its entirety, including: 2,6-Difluoro-N-(6-(5-(4-methyl-5-oxo-4,5-dihydro-1,3,4-oxadiazol-2-yl)-3-(trifluoromethyl)-1H-pyrazol-1-yl)pyridin-3-yl)benzamide; 2-Fluoro-6-methyl-N-(6-(5-(4-methyl-5-oxo-4,5-dihydro-1,3,4-oxadiazol-2-yl)-3-(trifluoromethyl)-1H-pyrazol-1-yl)pyridin-3-yl)benzamide; 5-(3-Cyclopropyl-1-(5-(2,6-difluorobenzyl)amino)pyridin-2-yl)-1H-pyrazol-5-yl)-3-methyl-1,3,4-oxadiazol-2(3H)-one; N-(6-(3-(Difluoromethyl)-5-(4-methyl-5-oxo-4,5-dihydro-1,3,4-oxadiazol-2-yl)-1H-pyrazol-1-yl)pyridin-3-yl)-2,6-difluorobenzamide; 5-(1-(5-(2,6-Difluorobenzyl)amino)pyridin-2-yl)-5-(fluoromethyl)-1H-pyrazol-3-yl)-3-methyl-1,3,4-oxadiazol-2(3H)-one; Methyl 3-(1-(5-((2,6-difluorobenzyl) amino)pyridin-2-yl)-5-(trifluoromethyl)-1H-pyrazol-3-yl)-5-methyl-4,5-dihydroisoxazole-5-carboxylate; Methyl 3-(1-(5-(2-chloro-6-fluorobenzyl)amino)pyridin-2-yl)-5-(trifluoromethyl)-1H-pyrazol-3-yl)-5-methyl-4,5-dihydroisoxazole-5-carboxylate; 2,6-Difluoro-W-(6-(3-(4-methyl-5-oxo-4,5-dihydro-1,3,4-oxadiazol-2-yl)-5-(trifluoromethyl)-1H-pyrazol-1-yl)pyridin-3-yl)benzamide; 2-Chloro-6-fluoro-N-(6-(3-(4-methyl-5-oxo-4,5-dihydro-1,3,4-oxadiazol-2-yl)-5-(trifluoromethyl)-\H-pyrazol-1-yl)pyridin-3-yl)benzamide; 2-Fluoro-6-methyl-/v-(6-(3-(4-methyl-5-oxo-4,5-dihydro-1,3,4-oxadiazol-2-yl)-5-(trifluoromethyl)-1H-pyrazol-1-yl)pyridin-3-yl)benzamide; N-(6-(5-(Difluoromethyl)-3-(4-methyl-5-oxo-4,5-dihydro-1,3,4-oxadiazol-2-yl)-1H-pyrazol-1-yl)pyridin-3-yl)-2,6-difluorobenzamide; 5-(1-(5((2,6-Difluorobenzyl) amino)pyridin-2-yl)-5-(difluoromethyl)-1H-pyrazol-3-yl)-3-methyl-1,3,4-oxadiazol-2(3H)-one; 5-(1-(5-((2,6-Difluorobenzyl)amino) pyridin-2-yl)-3-(difluoromethyl)-1H-pyrazol-5-yl)-3-methyl-1,3,4-oxadiazol-2(3H)-one; N-(6-(3-(5,5-Dimethyl-4-oxo-4,5-dihydroisoxazol-3-yl)-5-(trifluoromethyl)-1H-pyrazol-1-yl)pyridin-3-yl)-2,6-difluorobenzamide; 2-Chloro-N-(6-(3-(5,5-dimethyl-4-oxo-4,5-dihydroisoxazol-3-yl)-5-(trifluoromethyl)-1H-pyrazol-1-yl)pyridin-3-yl)-6-fluorobenzamide; 2,6-Difluoro-N-(6-(1′,4′,4,-trimethyl-5′-oxo-5-(trifluoromethyl)-4′,5′-dihydro-1H,1H′-[3,3′-bipyrazol]-1-yl)pyridin-3-yl)benzamide; 2-Chloro-6-fluoro-N-(6-(1,4,4′-trimethyl-5′-oxo-5-(trifluoromethyl)-4′,5,-dihydro-1H,1′H-[3,3′-bipyrazol]-1-yl)pyridin-3-yl)benzamide; 2-Fluoro-6-methyl-N-(6-(1,4,4′-trimethyl-5′-oxo-5-(trifluoromethyl)-4′,5′-dihydro-1H,1′H-[3,3′-bipyrazol]-1-yl)pyridin-3-yl)benzamide; 2,6-Difluoro-N-(6-(3-(4-methyl-5-oxo-4,5-dihydro-1,2,4-oxadiazol-3-yl)-5-(trifluoromethyl)-1H-pyrazol-1-yl)pyridin-3-yl)benzamide; N-(6-(3-(4-Acetyl-5,5-dimethyl-4,5-dihydro-1,3,4-oxadiazol-2-yl)-5-trifluoromethyl)-1H-pyrazol-1-yl)pyridin-3-yl)-2,6-difluorobenzamide; N-(6-(3-(4,4-Dimethyl-4,5-dihydrooxazol-2-yl)-5-(trifluoromethyl)-1H-pyrazol-1-yl)pyridin-3-yl)-2,6-difluorobenzamide; 5-(1-(5-(2,6-Difluorobenzyl)amino)pyridin-2-yl)-5-(trifluoromethyl)-1H-pyrazol-3-yl)-3-methyl-1,3,4-oxadiazol-2(3H)-one; 5-(1-(5-((2-Chloro-6-fluorobenzyl)amino)pyridin-2-yl)-5-(trifluoromethyl)-1H-pyrazol-3-yl)-3-methyl-1,3,4-oxadiazol-2(3iy)-one; 1′454(2,6-Difluorobenzyl)amino)pyridin-2-yl)-1,4,4-trimethyl-5′-(trifluoromethyl)-1H,1′H-[3,3′-bipyrazol]-5(4H)-one; 1′-(5-(2-Chloro-6-fluorobenzyl)amino)pyridin-2-yl)-1,4,4-trimethyl-5′-(trifluoromethyl)-1H,1′H-[3,3′-bipyrazol]-5(4H)-one; 3-(1-(5-(2,6-Difluorobenzyl)amino)pyridin-2-yl)-5-(trifluoromethyl)-1H-pyrazol-3-yl)-4-methyl-1,2,4-oxadiazol-5(4H)-one; 1-(5-(1-(5-(2,6-Difluorobenzyl)amino)pyridin-2-yl)-5-(trifluoromethyl)-1H-pyrazol-3-yl)-2,2-dimethyl-1,3,4-oxadiazol-3(2H)-yl)ethanone; N-(2,6-Difluorobenzyl)-6-(3-(4,4-dimethyl-4,5-dihydrooxazol-2-yl)-5-(trifluoromethyl)-1H-pyrazol-1-yl)pyridin-3-amine; N-(6-(5-Cyclopropyl-3-(4-methyl-5-oxo-4,5-dihydro-1,3,4-oxadiazol-2-yl)-1H-pyrazol-1-yl)pyridin-3-yl)-2,6-difluorobenzamide; N-(6-(3-Cyclopropyl-5-(4-methyl-5-oxo-4,5-dihydro-1,3,4-oxadiazol-2-yl)-1H-pyrazol-1-yl)pyridin-3-yl)-2,6-difluorobenzamide; 2,6-Difluoro-N-(6-(5-methyl-3-(4-methyl-5-oxo-4,5-dihydro-1,3,4-oxadiazol-2-yl)-1H-pyrazol-1-yl)pyridin-3-yl)benzamide; 5-(1-(5-((2,6-Difluorobenzyl) amino)pyridin-2-yl)-5-methyl-1H-pyrazol-3-yl)-3-methyl-1,3,4-oxadiazol-2(3H)-one; (3-(I-(5-((2,6-Difluorobenzyl)amino)pyridin-2-yl)-5-(trifluoromethyl)-1H-pyrazol-3-yl)-5-methyl-4,5-dihydroisoxazol-5-yl)methanol; (3-(1-(5-((2-Chloro-6-fluorobenzyl)amino)pyridin-2-yl)-5-(trifluoromethyl)-1H-pyrazol-3-yl)-5-methyl-4,5-dihydroisoxazol-5-yl)methanol; Methyl 3-(1-(5-(2,6-difluorobenzamido)pyridin-2-yl)-5-(trifluoromethyl)-1H-pyrazol-3-yl)-5-methyl-4,5-dihydroisoxazole-5-carboxylate; 2,6-Difluoro-N-(6-(3-(5-(hydroxymethyl)-5-methyl-4,5-dihydroisoxazol-3-yl)-5-(trifluoromethyl)-1H-pyrazol-1-yl)pyridin-3-yl)benzamide; 3-(1-(5-(2,6-Difluorobenzamido)pyridin-2-yl)-5-(trifluoromethyl)-1H-pyrazol-3-yl)-5-methyl-4,5-dihydroisoxazole-5-carboxamide; 2,6-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Chloro-6-fluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Fluoro-6-methyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Fluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,3-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,4,5-Trifluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,3,4-Trifluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,4-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2,3-Dimethyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Chloro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 2-Methyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide; 4-Ethyl-N-(5-(3-(4-methyl-5-oxo-4,5-dihydro-1,3,4-oxadiazol-2-yl)-5-(trifluoromethyl)-1//-pyrazol-1-yl)pyridin-2-yl)benzamide; N-(5-(3-(4-Methyl-5-oxo-4,5-dihydro-13,4-oxadiazol-2-yl)-5-(trifluoromethyl)-1H-pyrazol-1-yl)pyridin-2-yl)-2-naphthamide; 5-(1-(6-((2,6-Difluorobenzyl)amino)pyridin-3-yl)-5-(trifluoromethyl)-1H-pyrazol-3-yl)-3-methyl-1,3,4-oxadiazol-2(3H)-one; 5-(1-(6-((2-Chloro-6-fluorobenzyl)amino)pyridin-3-yl)-5-(trifluoromethyl)-1H-pyrazol-3-yl)-3-methyl-1,3,4-oxadiazol-2(3H)-one; 5-(1-(6-(2-Fluoro-6-methylbenzyl)amino)pyridm-3-yl)-5-(trifluoromethyl)-1H-pyrazol-3-yl)-3-methyl-1,3,4-oxadiazol-2(3H)-one; N-(2,6-Difluorophenyl)-6-(3-(4-methyl-5-oxo-4,5-dihydro-1,3,4-oxadiazol-2-yl)-5-(trifluoromethyl)-1H-pyrazol-1-yl)nicotinamide; N-(2-Chloro-6-fluorophenyl)-6-(3-(4-methyl-5-oxo-4,5-dihydro-1,3,4-oxadiazol-2-yl)-5-(trifluoromethyl)-1H-pyrazol-1-yl)nicotinamide;

a compound of formula (VIII):

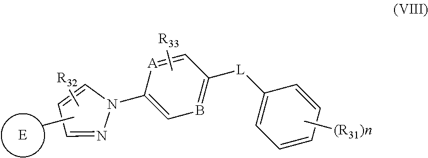

wherein: one of A and B is N and the other is CR₃₃; L is selected from —C(O)NR₁₁—, —NR₁₁C(O)—, —CRₐ₁R_{b1}NR₁₁— and NR₁₁CRₐ₁R_{b1}—; at each occurrence, Rₐ₁ and R_{b1} are independently hydrogen, substituted or unsubstituted alkyl or halogen; ring E is 5 membered non aromatic heterocyclic ring selected from Formula (a) to (c)

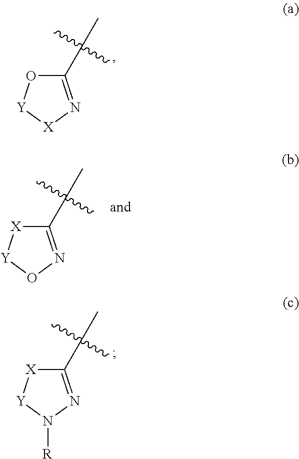

at each occurrence, X is selected from —C(O)—, —CR₃₄R₃₅— and —NR—; at each occurrence, Y is —C(O)— or —CR₃₄R₃₅—; provided that both of X and Y are not simultaneously —C(O)—; R is selected from substituted or unsubstituted alkyl, substituted or unsubstituted haloalkyl, substituted or unsubstituted cycloalkyl, substituted or unsubstituted cycloalkenyl, substituted or unsubstituted aryl, substituted or unsubstituted heteroaryl, substituted or unsubstituted heterocyclyl, —C(O)NR₃₆R₃₇, —C(O)OR₃₉ and —C(O)R₃₈; R₃₁, which may be same or different at each occurrence, is independently selected from halogen, cyano, nitro, hydroxyl, substituted or unsubstituted alkyl, substituted or unsubstituted haloalkyl, substituted or unsubstituted alkoxy, substituted or unsubstituted haloalkoxy, substituted or unsubstituted cycloalkyl, substituted or unsubstituted cycloalkoxy, —NR₃₆R₃₇, —NHC(O) R₃₈, and —C(O)OR₃₉; or any two of adjacent R₃₁ groups together with the phenyl to which they are attached form substituted or unsubstituted naphthalene ring; R₃₂ is selected from halogen, cyano, nitro, hydroxyl, substituted or unsubstituted alkyl, substituted or unsubstituted haloalkyl, substituted or unsubstituted alkoxy, substituted or unsubstituted haloalkoxy, substituted or unsubstituted cycloalkyl, substituted or unsubstituted cycloalkoxy, —NR₃₆R₃₇, —NHC(O) R₃₈, and —C(O)OR₃₉; R₃₃ is selected from hydrogen, halogen, cyano, nitro, hydroxyl, substituted or unsubstituted alkyl, substituted or unsubstituted haloalkyl, substituted or unsubstituted alkoxy, substituted or unsubstituted haloalkoxy, substituted or unsubstituted cycloalkyl, substituted or unsubstituted cycloalkoxy, —NR₃₆R₃₇, —NHC(O) R₃₈, and —C(O)OR₃₉; R₃₄ and R₃₅, which may be same or different at each occurrence, are independently selected from hydrogen, halogen, —OR₁₀, substituted or unsubstituted alkyl, substituted or unsubstituted haloalkyl, substituted or unsubstituted hydroxyalkyl, —C(O)OR₃₆, —C(O)—NR₃₆R₃₇, substituted or unsubstituted cycloalkyl, substituted or unsubstituted aryl, substituted or unsubstituted heteroaryl and substituted or unsubstituted heterocyclyl; provided that, when any of R₃₄ or R₃₅ in Y is —OR₁₀ then R₁₀ is not hydrogen; R₃₆ and R₃₇, which may be same or different at each occurrence, are independently selected from hydrogen, substituted or unsubstituted alkyl and substituted or unsubstituted cycloalkyl; or R₃₆ and R₃₇, together with the nitrogen atom to which they are attached, may form a substituted or unsubstituted, saturated or unsaturated 3 to 12 membered cyclic ring, wherein the unsaturated cyclic ring may have one or two double bonds; R₃₈, which may be same or different at each occurrence, is independently selected from substituted or unsubstituted alkyl, substituted or unsubstituted alkenyl, substituted or unsubstituted cycloalkyl, and substituted or unsubstituted aryl; R₃₉, which may be same or different at each occurrence, is independently selected from hydrogen, substituted or unsubstituted alkyl and substituted or unsubstituted aryl; R₁₀ is selected from hydrogen, substituted or unsubstituted alkyl, substituted or unsubstituted cycloalkyl, substituted or unsubstituted aryl, substituted or unsubstituted heteroaryl and substituted or unsubstituted heterocyclyl; at each occurrence, Rᵢᵢ is independently hydrogen or substituted or unsubstituted alkyl; and n is an integer ranging from 0 to 4, both inclusive; or a pharmaceutically acceptable salt thereof;

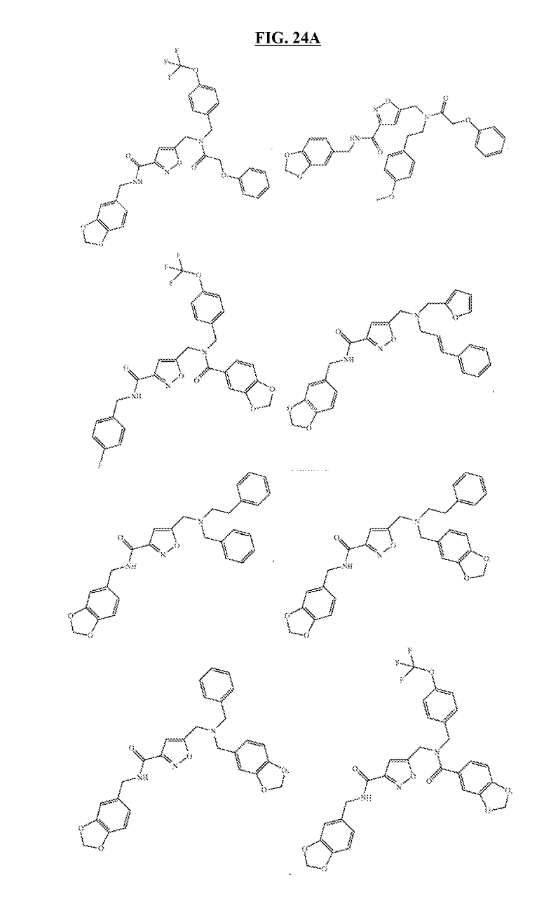

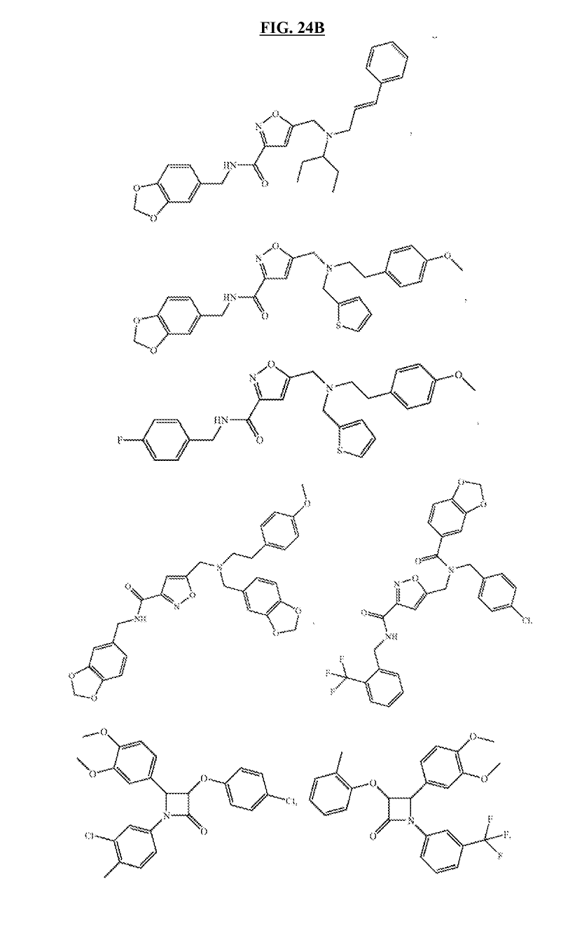

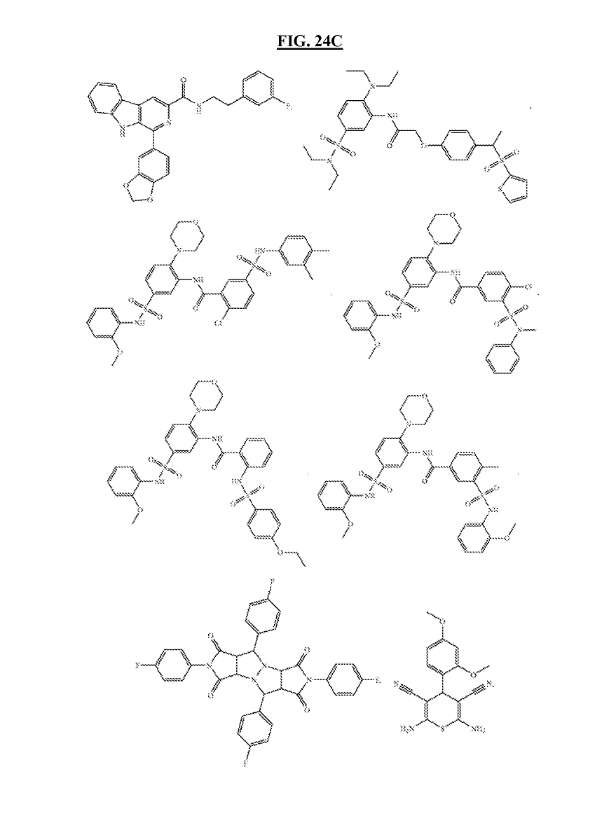

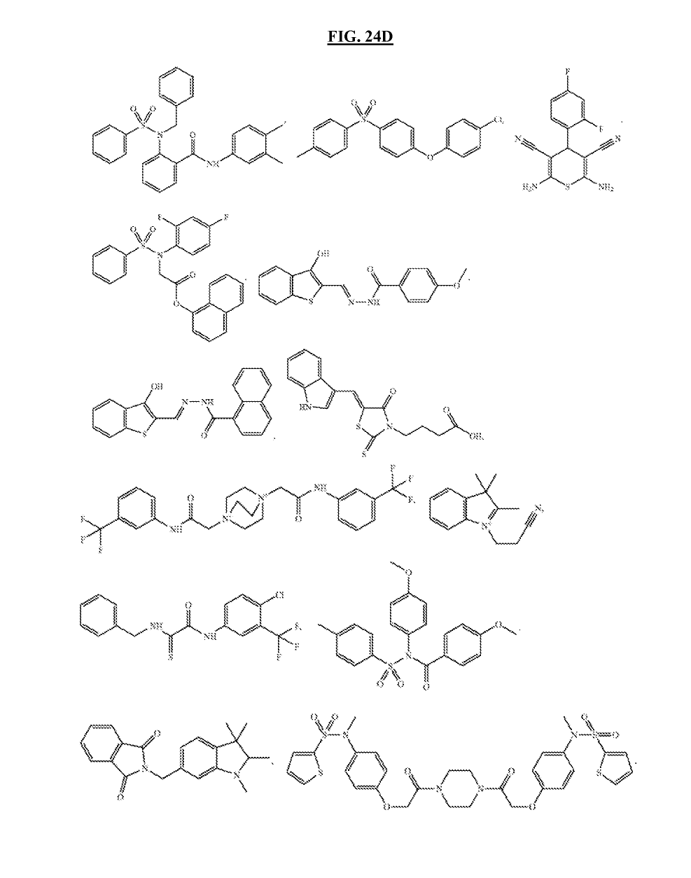

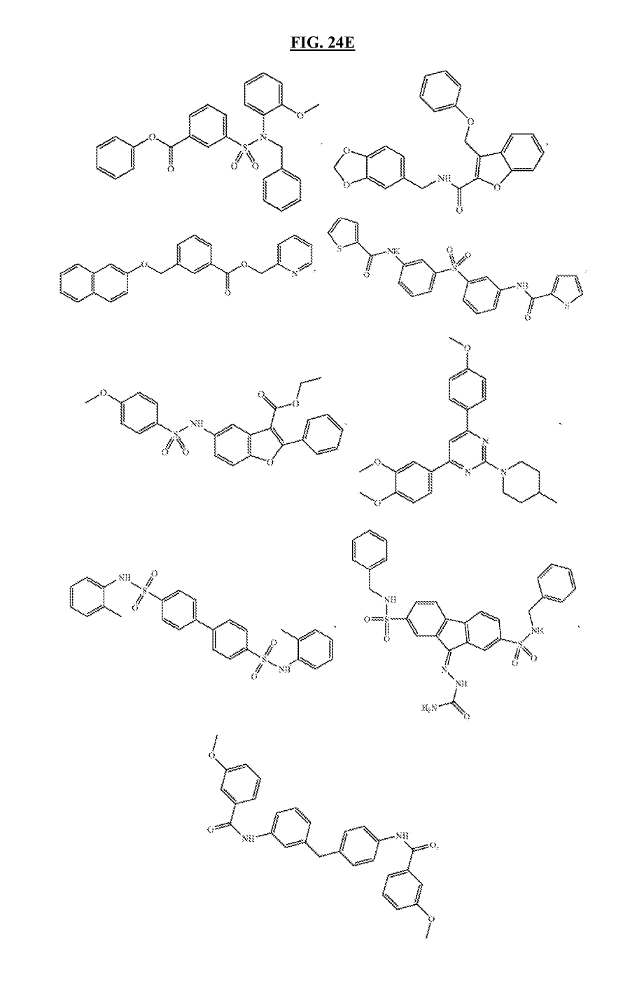

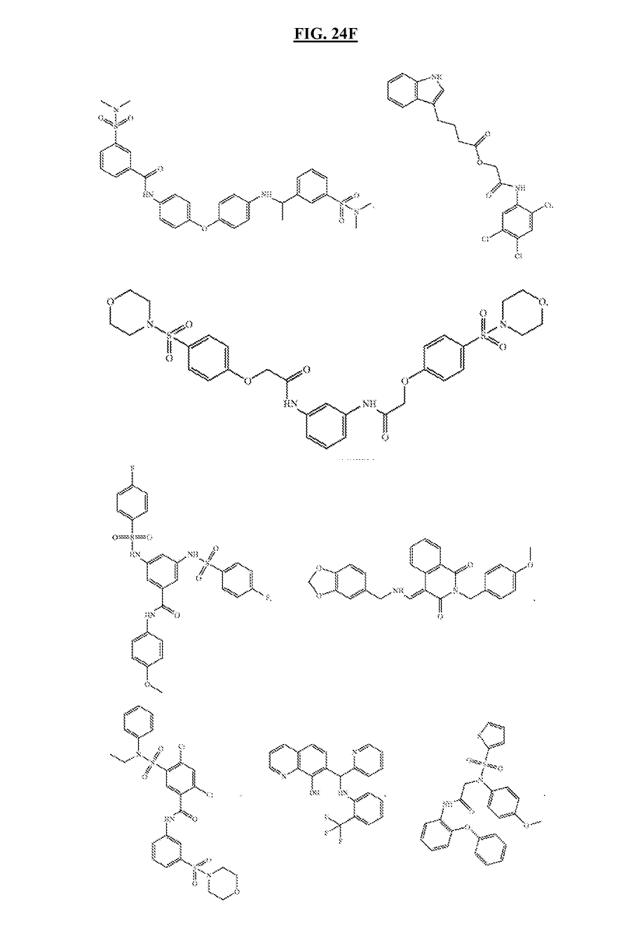

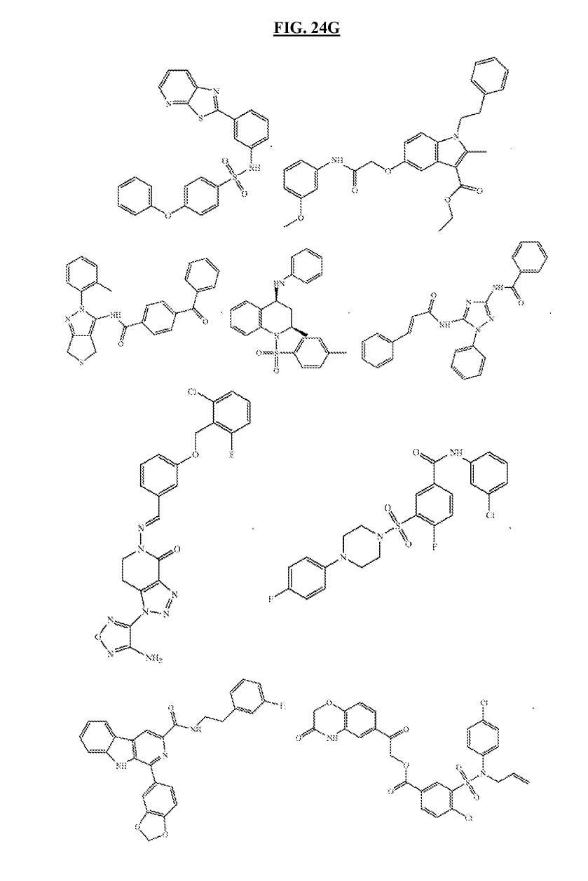

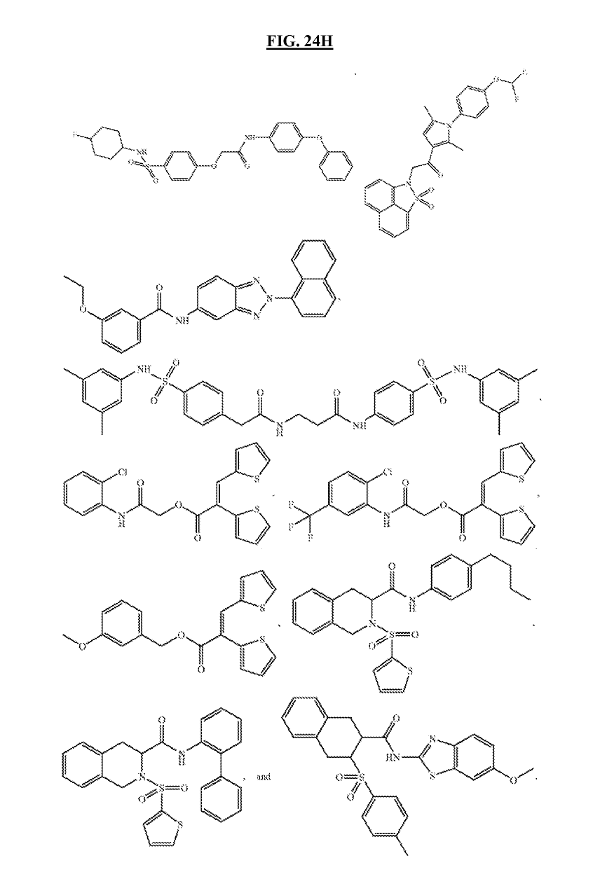

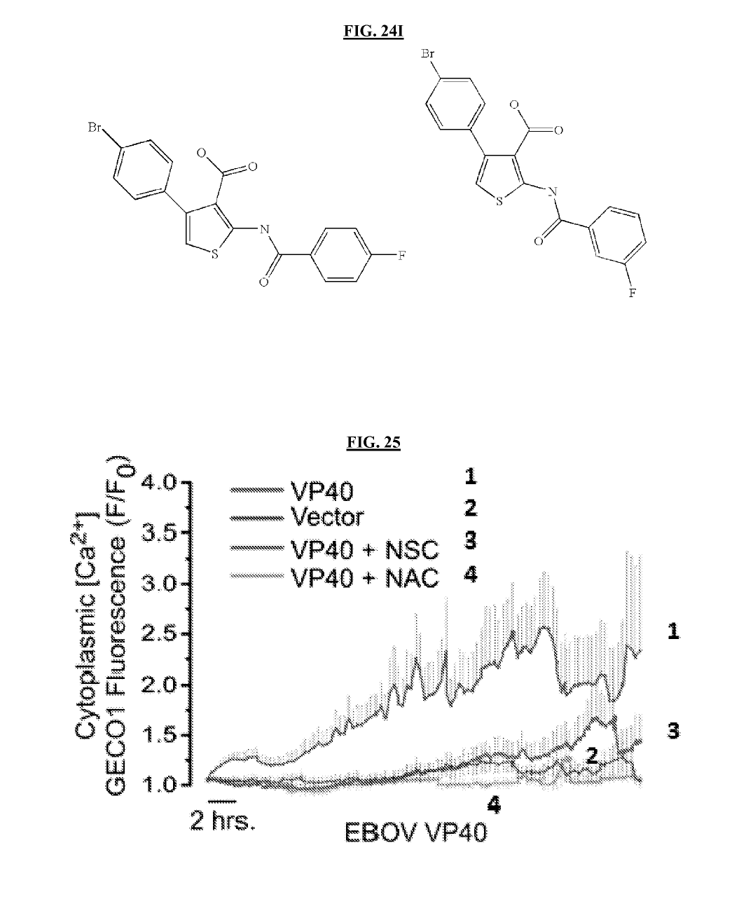

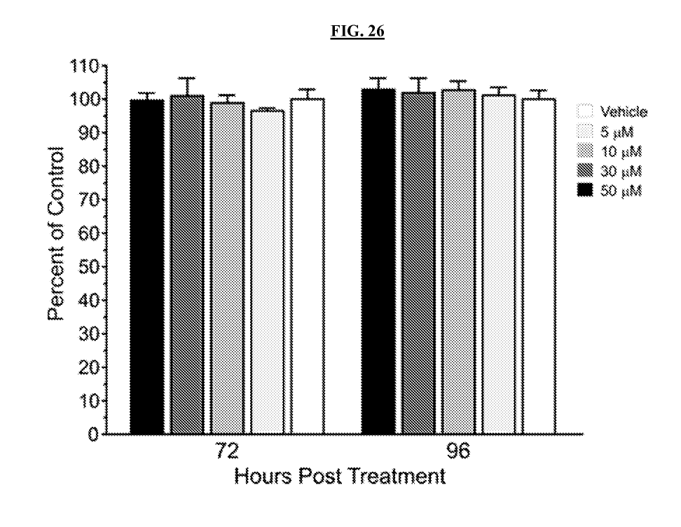

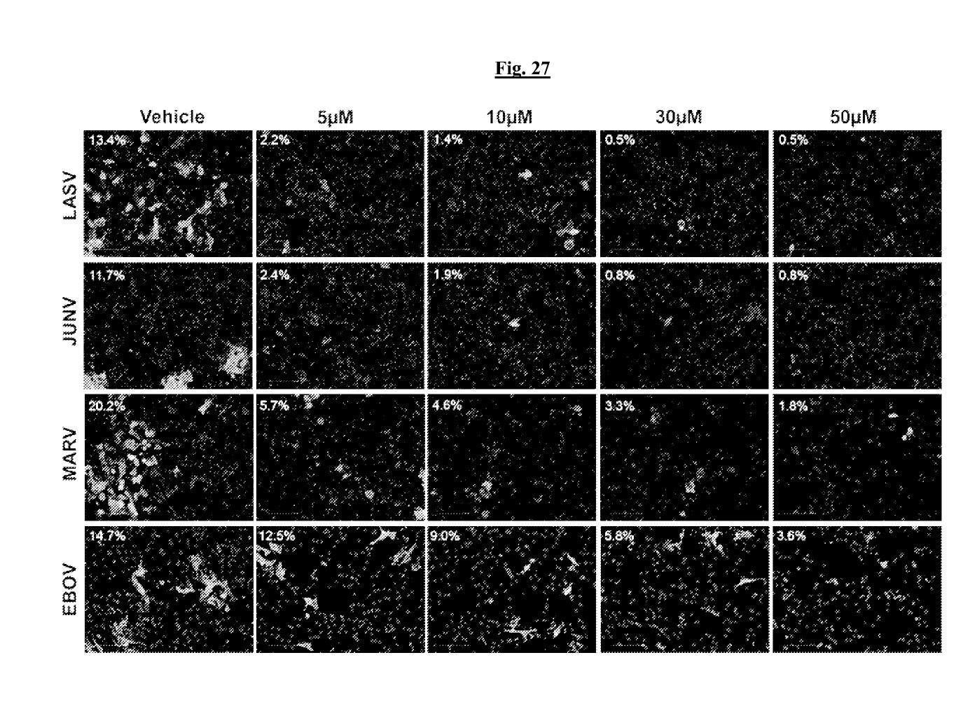

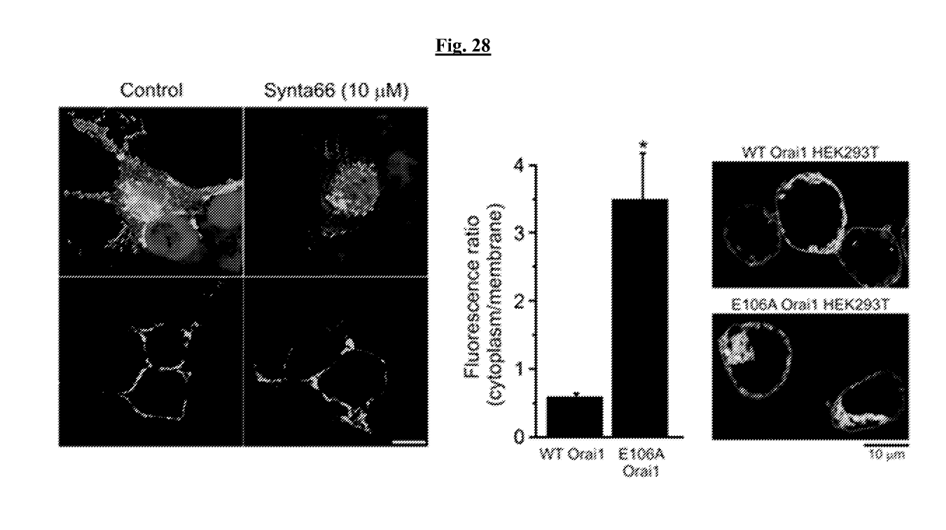

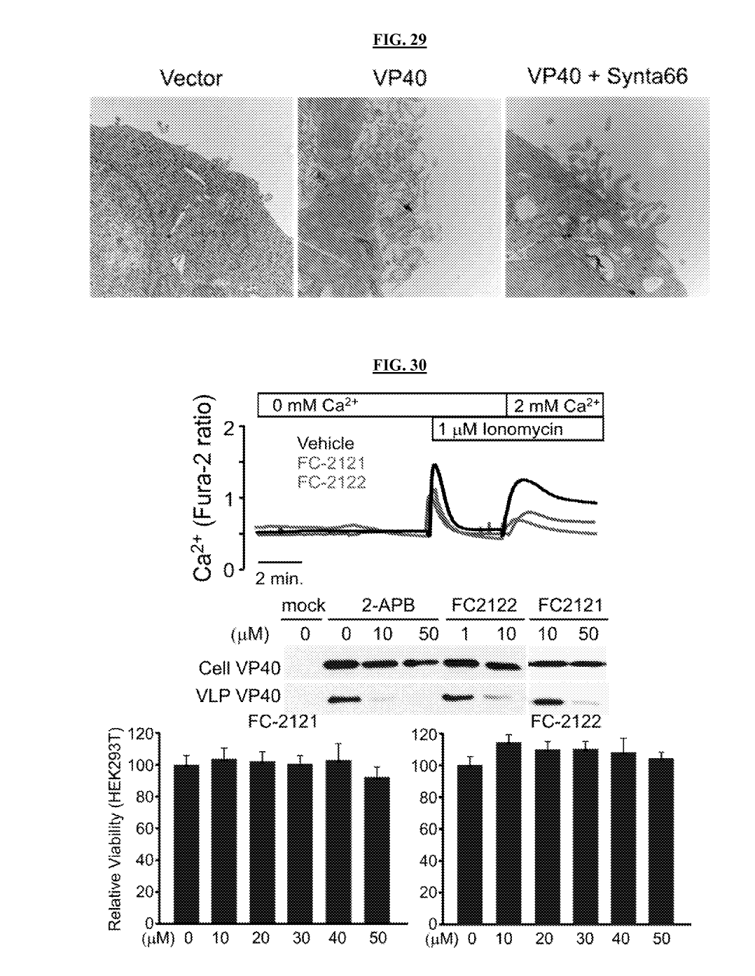

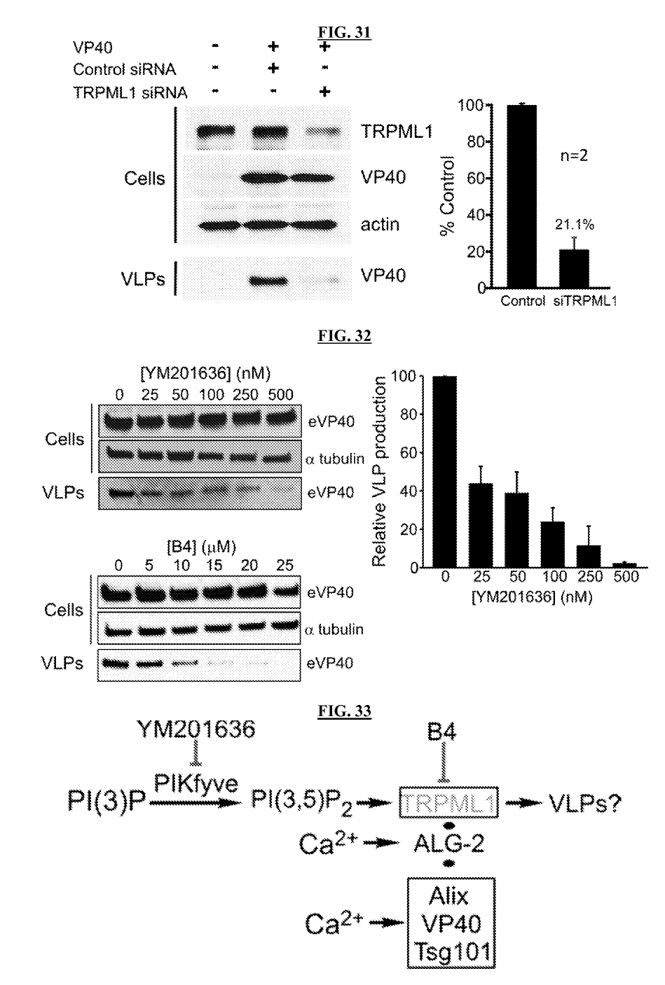

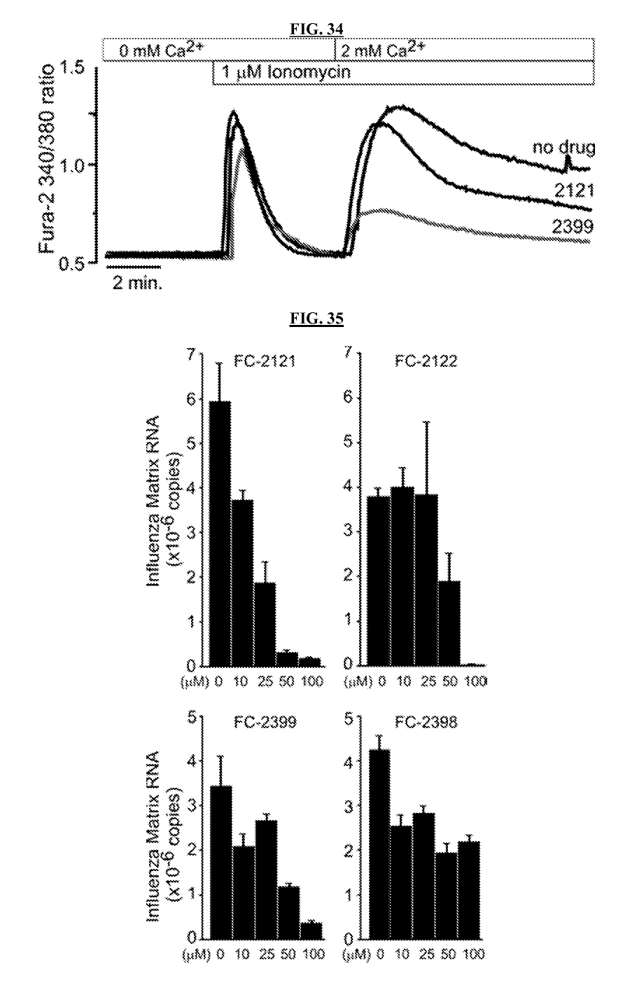

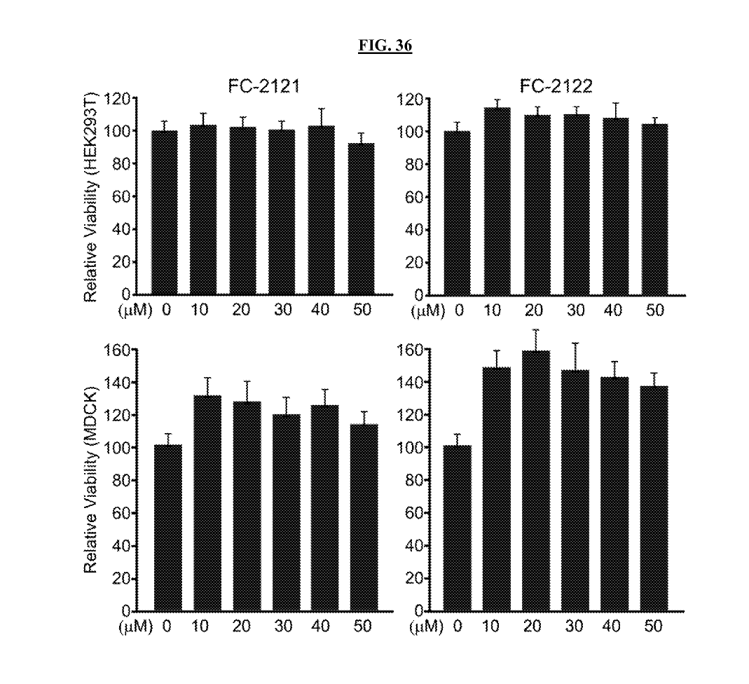

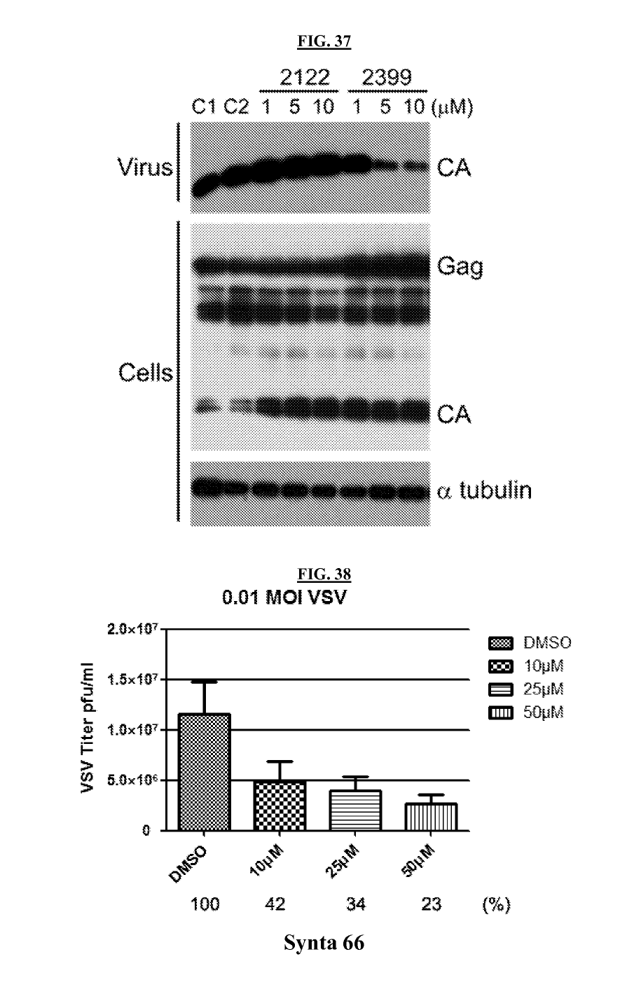

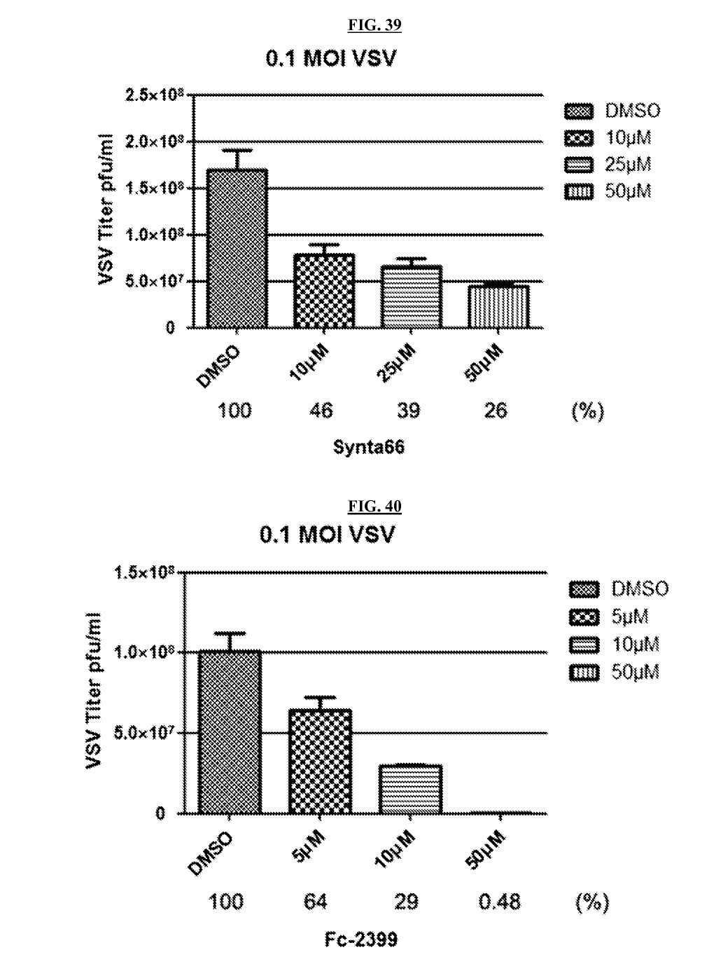

the compounds disclosed in U.S. Patent Application Publication No. US2010/0087415, which entire disclosure is incorporated herein by reference in its entirety, including the compounds illustrated in FIG. 24A-24I;

a compound of formula (IX):

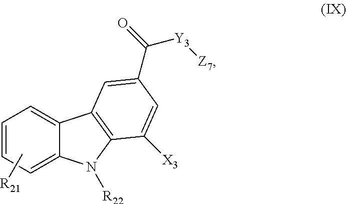

wherein: X₃ is cycloalkyl, heterocycloalkyl, aryl, or heteroaryl wherein cycloalkyl, heterocycloalkyl, aryl, or heteroaryl is optionally substituted with at least one R₃; Y₃ is a bond, C₁-C₆alkyl, C₂-C₆alkenyl, NR₂, O, S, NR₂(C₁-C₆alkyl), O(C₁-C₆alkyl), S(C₁-C₆alkyl), NR₂(C₂-C₆alkenyl), O(C₂-C₆alkenyl), S(C₂-C₆alkenyl); wherein C₁-C₆alkyl or C₂-C₆alkenyl are optionally substituted with at least one R₃; Z₇ is cycloalkyl, heterocycloalkyl, aryl, or heteroaryl wherein cycloalkyl, heterocycloalkyl, aryl, or heteroaryl is optionally substituted with at least one R4; each R₂₁ is independently selected from H, F, Cl, Br, I, —CN, —NO₂, —OH, —CF₃, —OCF₃, —OR₉, C₁-C₆alkyl, C₃-C₈cycloalkyl, C₁-C₆heteroalkyl, C₁-C₆haloalkyl, C₂-C₈heterocycloalkyl, optionally substituted aryl, optionally substituted O-aryl, optionally substituted heteroaryl, —NHS(═O)₂R₈, —S(═O)₂N(R₉)₂, —N(R₉)S(═O)₂N(R₉)₂, —C(═O)CF₃, —C(═O)NHS(═O)₂R₈, —S(═O)₂NHC(═O)R₈, —N(R₉)₂, —N(R₉)C(═O)R₈, —N(R₉)C(═O)N(R₉)₂, —N(R₉)C(═O)OR₈, —CO₂R₉, —C(═O)R₈, —OC(═O)R₈, —OC(═O)N(R₉)₂, —CON(R₉)₂, —SR₈, —S(═O)R₈, and —S(═O)₂R₈; R₂₂ is H, C₁-C₆alkyl, and C₃-C₈cycloalkyl; R₃ and R₄ are each independently selected from F, Cl, Br, I, —CN, —NO₂, —OH, —CF₃, —OCF₃, —OR₉, C₁-C₆alkyl, C₃-C₈cycloalkyl, C₁-C₆heteroalkyl, C₁-C₆haloalkyl, C₂-C₈heterocycloalkyl, optionally substituted aryl, optionally substituted O-aryl, optionally substituted heteroaryl, —NHS(═O)₂R₈, —S(═O)₂N(R₉)₂, —N(R₉)S(═O)₂N(R₉)₂, —C(═O)CF₃, —C(═O)NHS(═O)₂R₈, —S(═O)₂NHC(═O)R₈, —N(R₉)₂, —N(R₉)C(═O)R₈, —N(R₉)C(═O)N(R₉)₂, —N(R₉)C(═O)OR₈, —CO₂R₉, —C(═O)R₈, —OC(═O)R₈, —OC(═O)N(R₉)₂, —CON(R₉)₂, —SR₈, —S(═O)R₈, and —S(═O)₂R₈; or two R₃ together with the atoms to which they are attached form a heterocycloalkyl group having at least one O, NH, or S; each R₈ is independently selected from C₁-C₆alkyl, C₁-C₆haloalkyl, C₃-C₈cycloalkyl, phenyl, and benzyl; each R₉ is independently selected from H, C₁-C₆alkyl, C₁-C₆haloalkyl, C₃-C₈cycloalkyl, phenyl, and benzyl; or a pharmaceutically acceptable salt, pharmaceutically acceptable prodrug, or pharmaceutically acceptable solvate thereof;

the compounds illustrated in Table 1;

the compounds disclosed in U.S. Patent Application Publication Nos. US2009137659, US2012289587, US2010305200, US2013123265, US2010056532, US2010261725, US2011136816, US2011166159, US2010152241, US2011257177, US2011269743, US2010087415, US2012149673, US2011230536, US2011065724, US2013079348, US2011212970, US2011263612, US2013345240, US2013345193, US2013203818, US2012053210, US2012071516, US2013143927, US2012316182, US2013245063, US2013245025, US2013231344, and US2014051711, and PCT Patent Application Publication Nos. WO2013059677, WO2013059666, WO2014043715, and WO2014059333, which entire disclosures are incorporated herein by reference in their entireties;

a compound of formula (I)

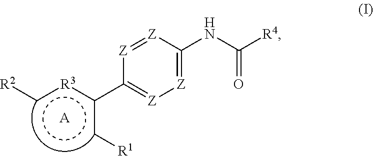

wherein in (I): ring A is a monocyclic or bicyclic cycloalkyl, heterocyclyl, aryl or heteroaryl ring; R¹ and R² are independently selected from the group consisting of optionally substituted C₁-C₁₀ alkyl, optionally substituted C₃-C₁₀ cycloalkyl, optionally substituted C₆-C₁₀ aryl, optionally substituted C₄-C₁₀ heterocyclyl, optionally substituted heteroaryl, halogen, —OH, alkoxy, —NH₂, —N(CH₃)₂, trifluoromethyl, —CN, —C(═O)OH, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂; R³ is CH, N, O or S; each occurrence of Z is independently CH or N; and R⁴ is optionally substituted C₃-C₁₀ cycloalkyl, optionally substituted C₆-C₁₀ aryl, optionally substituted C₄-C₁₀ heterocyclyl, or optionally substituted heteroaryl;

The compounds of the invention may possess one or more stereocenters, and each stereocenter may exist independently in either the (R) or (S) configuration. In one embodiment, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds recited herein.

In one embodiment, compounds described herein are prepared as prodrugs. A “prodrug” is an agent converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The invention further includes a pharmaceutical composition comprising the compound of the invention and a pharmaceutically acceptable carrier.

In certain embodiments, the pharmaceutical composition further comprises at least one additional agent that is useful to treat the diseases or disorders contemplated herein. In certain embodiments, the compound of the invention and the additional agent are co-formulated in the composition.

### Salts

The compounds described herein may form salts with acids or bases, and such salts are included in the present invention. In one embodiment, the salts are pharmaceutically acceptable salts. The term “salts” embraces addition salts of free acids or bases that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, fl-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

### Combination Therapies

In certain embodiments, the compounds of the invention are useful in the methods of the invention in combination with at least one additional compound useful for treating or preventing a disease or disorder contemplated within the invention. This additional compound may comprise compounds identified herein or compounds, e.g., commercially available compounds, known to treat, prevent or reduce the symptoms of a viral infection.

In certain embodiments, the at least one additional compound is an antiviral agent.

In certain embodiments, the compounds useful within the invention may be used in combination with one or more of the following anti-HIV drugs:

HIV Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fumarate (Truvada®/Gilead).

Entry and Fusion Inhibitors: maraviroc (Celsentri®, Selzentry®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddl, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil fumarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

In certain embodiments, the compounds of the invention may be used in combination with one or more voltage dependent calcium (VDC) channel inhibitors, such as but not limited to diltiazem, nifedipine, and gabapentin. In other embodiments, VDC channel inhibitors inhibit viral entry. In yet other embodiments, the combination of VDB channel inhibitors and CRAC channel inhibitors inhibit virus entry and egress from cells.

In certain embodiments, the compounds of the invention may be used in combination with the compounds, or a salt or solvate thereof:

2-((1,1-dioxidobenzo[d]isothiazol-3-yl)amino)phenyl 2-chlorobenzoate), as recited in Lu, et al., 2014, J. Virol. 88(9):4736

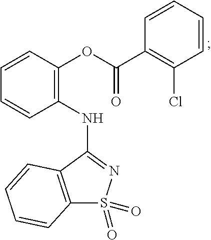 [SMILES: O=C(Oc1ccccc1NC1=NS(=O)(=O)c2ccccc21)c1ccccc1Cl]

Amb123203 (3-(2-Benzothiazolyl)-N-(3,5-dimethyl-1-phenyl-1H-pyrazol-4-yl)-1-piperidineacetamide), Han, et al., 2014. J. Virol. 88(13):7294

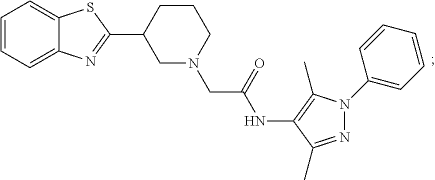 [SMILES: Cc1nn(-c2ccccc2)c(C)c1NC(=O)CN1CCCC(c2nc3ccccc3s2)C1]

Amb21795397 (1-[2-(3-Methyl-quinoxalin-2-ylsulfanyl)-acetyl]-3-phenyl-urea), Han, et al., 2014. J. Virol. 88(13):7294

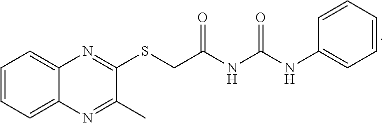 [SMILES: Cc1nc2ccccc2nc1SCC(=O)NC(=O)Nc1ccccc1]

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Methods

In one aspect, the invention includes a method of treating or preventing viral infection in a subject in need thereof. In certain embodiments, the method comprises administering to the subject a therapeutically effective amount of a pharmaceutically acceptable composition comprising at least one compound of the invention. In other embodiments, the viral infection is caused by at least one virus selected from the group consisting of a filovirus, arenavirus, rhabdovirus, orthomyxovirus, paramyxovirus, retrovirus, and any combinations thereof.

In certain embodiments, the composition is administered to the subject by at least one route selected from oral, rectal, mucosal (e.g., by oral or nasal inhalation), transmucosal, topical (transdermal), or by intravenous, intradermal, intramuscular, subcutaneous, intracutaneous, intrauterine, epidural or intracerebroventricular injection. In other embodiments, the subject is further administered at least one additional compound useful for treating or preventing a viral infection. In yet other embodiments, the subject is a mammal. In yet other embodiments, the mammal is human. In yet other embodiments, the subject is not responsive to one or more commercially available antivirals.

### Formulations/Administration

The compositions of the present invention may contain a pharmaceutical acceptable carrier, excipient and/or diluent, and may be administered by a suitable method to a subject. The compositions of the present invention may be formulated in various forms, including oral dosage forms or sterile injectable solutions, according to any conventional method known in the art. In other embodiments, the compositions may also be used as an inhalation-type drug delivery system. In yet other embodiments, the compositions of the invention may be formulated for injectable solutions.

The compositions may be formulated as powders, granules, tablets, capsules, suspensions, emulsions, syrup, aerosol, preparations for external application, suppositories and sterile injectable solutions. Suitable formulations known in the art are disclosed in, for example, Remington's Pharmaceutical Science (Mack Publishing Company, Easton Pa.). Carriers, excipients and diluents that may be contained in the composition of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propyl hydroxylbenzoate, talc, magnesium stearate or mineral oil.

Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here. Capsule formulations may be of the hard gelatin or soft gelatin variety and can contain the active component in solid, semi-solid, or liquid form. Gelatin capsules can be formed from animal gelatin or synthetic or plant derived equivalents thereof. The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum. Alternatively or additionally, the coating can be used as a taste masking agent to mask unpleasant tastes such as bitter tasting drugs. The coating may contain sugar or other agents that assist in masking unpleasant tastes. Instead of, or in addition to, a coating, the antibiotic can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g., a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art. The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions. Each tablet, capsule, caplet, pill, etc. can be a single dose, with a dose, for example, as herein discussed, or a dose can be two or more tablets, capsules, caplets, pills, etc; for example if a tablet, capsule etc is 125 mg and the dose is 250 mg, the patient may take two tablets, capsules and the like, at each interval there is to administration.

The compositions of the present invention may be formulated with commonly used diluents or excipients, such as fillers, extenders, binders, wetting agents, disintegrants, or surfactants. Solid formulations for oral administration include tablets, pills, powders, granules, or capsules, and such solid formulations comprise, in addition to the composition, at least one excipient, for example, starch, calcium carbonate, sucrose, lactose or gelatin. In addition to simple excipients, lubricants such as magnesium stearate or talc may also be used. Liquid formulations for oral administration include suspensions, solutions, emulsions and syrup, and may contain various excipients, for example, wetting agents, flavoring agents, aromatics and preservatives, in addition to water and liquid paraffin, which are frequently used simple diluents.

Formulations for parenteral administration include sterilized aqueous solutions, non-aqueous solutions, suspensions, emulsions, freeze-dried preparations, and suppositories. As non-aqueous solvents or suspending agents, propylene glycol, polyethylene glycol, plant oils such as olive oil, or injectable esters such as ethyl oleate may be used. As the base of the suppositories, witepsol, Macrogol, Tween 61, cacao butter, laurin fat, or glycerogelatin may be used.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

The compositions of the present invention may be administered to a subject by various routes. All modes of administration are contemplated, for example, orally, rectally, mucosally (e.g., by oral or nasal inhalation), transmucosally, topically (transdermal), or by intravenous, intradermal, intramuscular, subcutaneous, intracutaneous, intrauterine, epidural or intracerebroventricular injection.

### Dosing

The preferred dose of the pharmaceutical compositions of the present invention varies depending on the patient's condition and weight, the severity of the disease, the type of drug, and the route and period of administration and may be suitably selected by those skilled in the art. For preferred effects, the pharmaceutical composition of the present invention may be administered at a dose of 0.01-100 mg/kg/day. The administration may be anywhere from 1 to 4 times daily, e.g., once, twice, three times or four times daily. The maximum amount administered in a 24 hour period may be up to 1,500 mg. The administration may be over a course of 2 to 30 days, e.g., 3 to 21 days, such as 7, 10 or 14 days. The skilled person can adjust dosing depending on the subject's body weight and overall health condition and the purpose for administering the compound. Repeated courses of treatment may be pursued depending on the response obtained. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all remaining starting materials were obtained from commercial suppliers and used without purification.

### Example 1: STIM1 and Orai-Mediated Calcium Entry

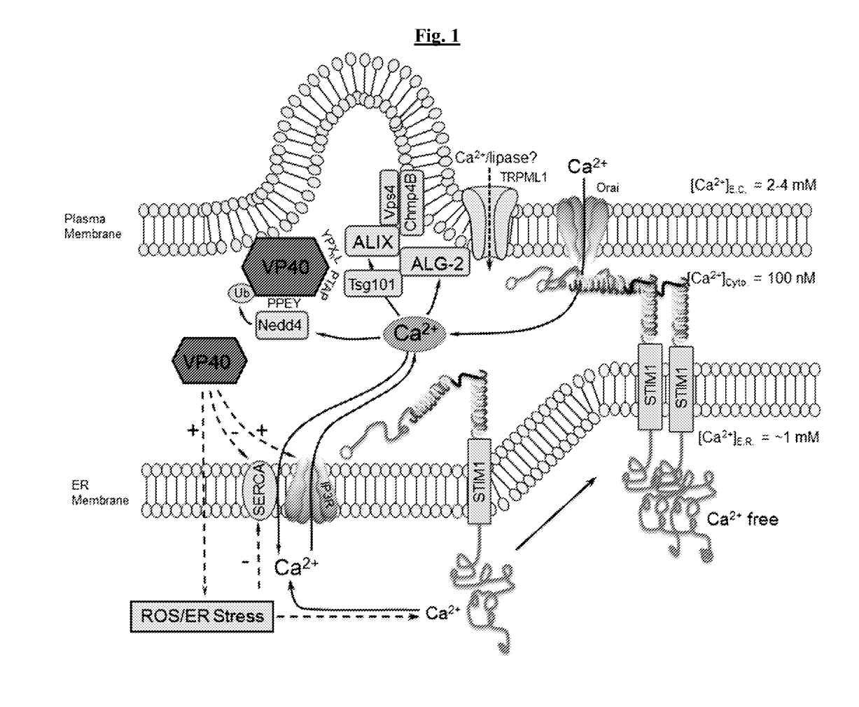

Store-operated calcium (SOC) entry represents a functionally critical mechanism of calcium entry in non-excitable cells. SOC is triggered classically by activation of tyrosine kinase or G-protein coupled receptors by cognate ligand. These receptors activate one of a number of phospholipase C isoforms, which hydrolyze the conversion of plasma membrane PIP2 into the second messengers diacylglycerol (DAG) and inositol trisphosphate (IP3). IP3 binds to and activates receptors (IP3Rs) on the ER membrane and calcium moves down its concentration gradient from the ER into the cytoplasm. This depletion of calcium in ER stores below the K_{d} for STIM EF hands induces STIM to change conformation, oligomerize, and relocalize to facilitate contact with and activation of CRAC channels in the PM. Host targets of enveloped RNA viruses including epithelial and other cells have a functional SOC mechanism, and the data presented herein demonstrate they critically depend upon this mechanism for efficient budding (FIG. 1).

In addition to activation by IP3, passive depletion of ER Ca²⁺ with drugs (e.g., thapsigargin or Tg) that inhibit SERCA (sarco-endoplasmic Ca²⁺ ATPase in the membrane) or ionophores that create calcium permeable pores in the ER (ionomycin), activate store-operated calcium entry. Experimentally, thapsigargin can be used to distinguish between changes in cytoplasmic calcium due to ER release versus entry through Orai channels.

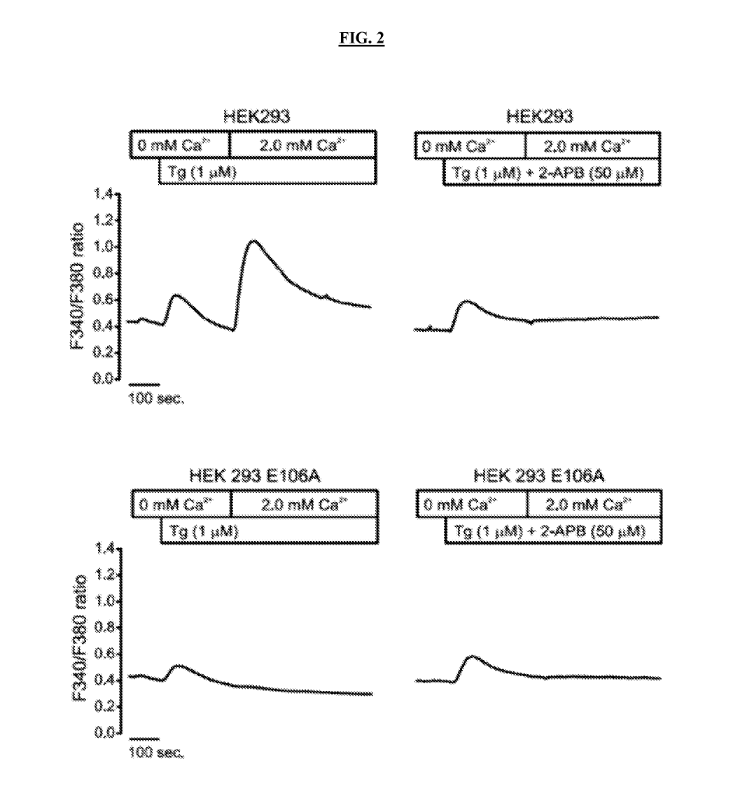

For example, HEK293 cells loaded with the cytosolic calcium indicator Fura-2 and bathed in calcium-free medium exhibited a transient increase in cytosolic calcium levels following thapsigargin application that reflects the net loss of calcium from the ER. The subsequent decay in concentration was due to PMCA (plasma membrane Ca²⁺ ATPase)-mediated Ca²⁺ extrusion from the cytoplasm. Following this initial transient calcium rise, superfusion with calcium-containing medium produced a secondary and sustained increase in cytosolic steady-state calcium concentration due to ion entry through activated CRAC/Orai1 channels and this was blocked by Orai inhibitors such as 2-APB or by expression of a dominant negative mutant of Orai1 (E106A) (FIG. 2).

### Example 2: VP40 Mobilizes Cellular Calcium

Without wishing to be limited by any theory, the findings demonstrating a requirement for STIM1 and Orai1 in both Ebola and Marburg VP40-mediated VLP formation have two potential mechanistic implications. One is that constitutive or homeostatic Orai1-mediated calcium signals are sufficient to support matrix protein-mediated budding, and the other is that VP40 directly or indirectly mobilizes calcium by activating STIM1 and then Orai1.

In certain embodiments, in the event that VP40 orchestrates budding via control of Ca²+-dependent host mechanisms, VP40 expression induces an increase in cytoplasmic calcium during the course of viral assembly and budding. To assess this hypothesis, time-dependent changes in cytosolic Ca²⁺ levels in eVP40-GFP were measured versus vector expressing WT HEK293 cells during the normal time course of VLP production. Calcium and VP40 expression levels were monitored from 6-24 hours after VP40-GFP (or control pCAGGS vector) transfection of cells co-expressing the genetically encoded calcium indicator GECO-1. VP40 GFP (green) and GECO-1 (red) fluorescence were monitored concurrently in vector and VP40 expressing cells cultured in adjacent wells of a chamber slide on the stage of a spinning disk confocal microscope enclosed in a temperature and CO₂ controlled environmental chamber.

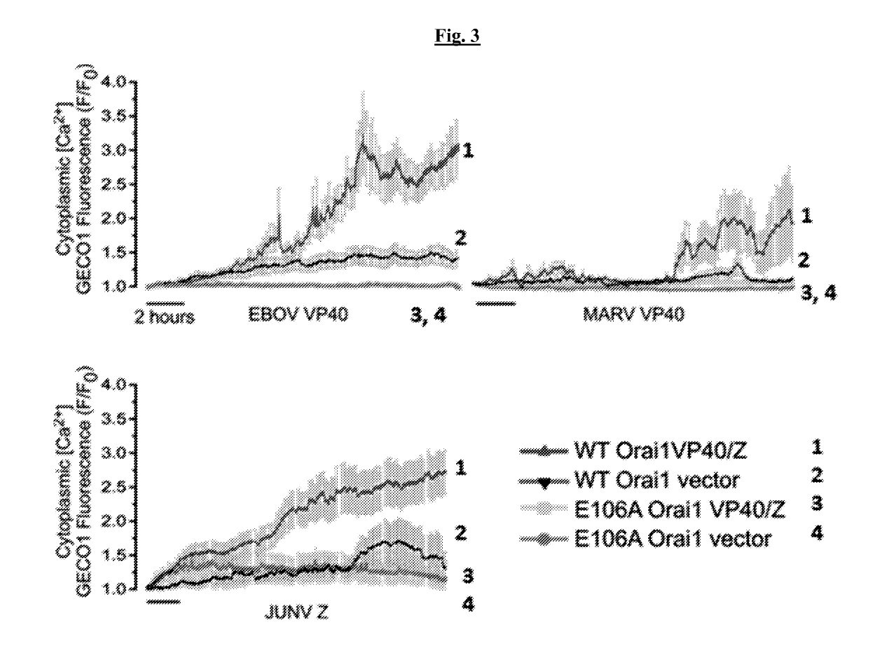

VP40 expressing cells exhibited a gradual and significant increase in cytosolic Ca²⁺ (FIG. 3, blue—top trace), while Ca²⁺ levels in vector treated WT (purple—middle trace) increased to a low steady state. However, no Ca²⁺ signal was evoked by VP40 or vector in E106A HEK293 cells over the same period. These results suggest that VP40 triggers STIM1 activation, and that the resulting Ca²⁺ signals regulate budding (see also FIG. 3).

### Example 3: Block in Calcium Entry by a Dominant Negative Orai1 Mutant, by STIM1 Suppression, or by Pharmacological Inhibition of Orai1

Studies were performed to identify proteins responsible for the regulation of Ebola VP40-mediated virus like particle (VLP) formation, as a prelude to investigating their role in late steps of virus budding.

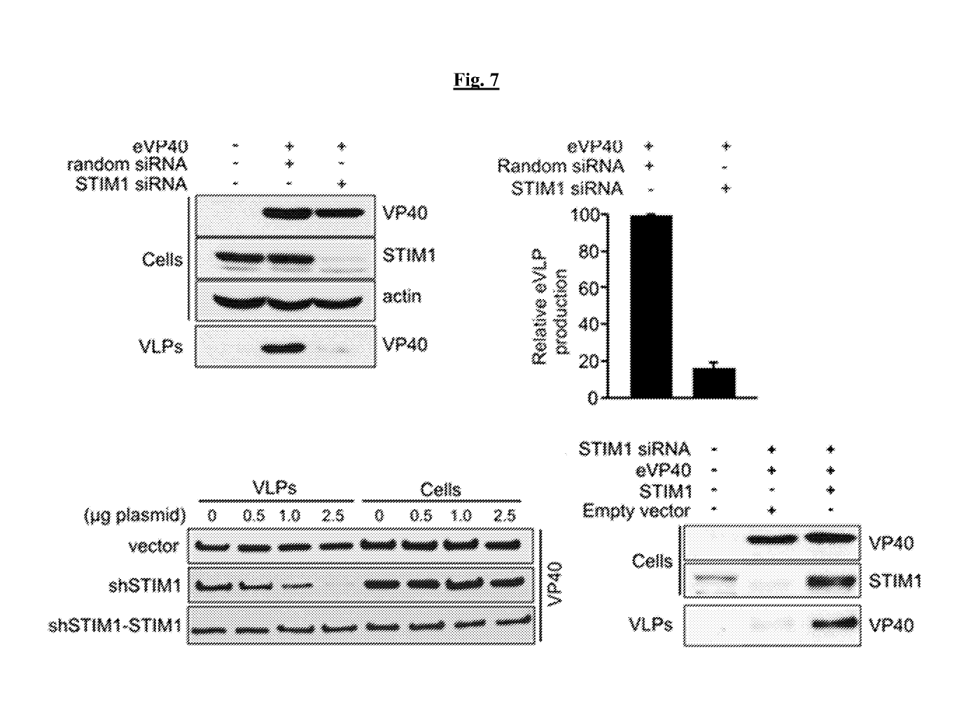

Three complementary approaches were implemented. In the first, the HEK293 cell line that stably overexpresses a dominant negative mutant of Orai1 (Orai1 E106A, FIG. 3) was utilized; this mutant incorporates into endogenous WT Orai hexameric channels and functionally inactivates them. A second approach involved a STIM1 shRNA construct and a companion bicistronic vector that encodes both a STIM1 shRNA that targets the endogenous STIM1 5′-UTR and simultaneously expresses STIM1 cDNA to rescue expression or siRNA and STIM1 cDNA to rescue expression (FIGS. 7A-7B). A third approach utilized CRAC channel inhibitors 2-aminoethoxydiphenyl borate (2-APB), Synta66, or R02959 to probe the role of CRAC in SOC entry. In all instances, the SERCA inhibitor Tg was used to deplete calcium from the ER and trigger STIM-Orai activation.

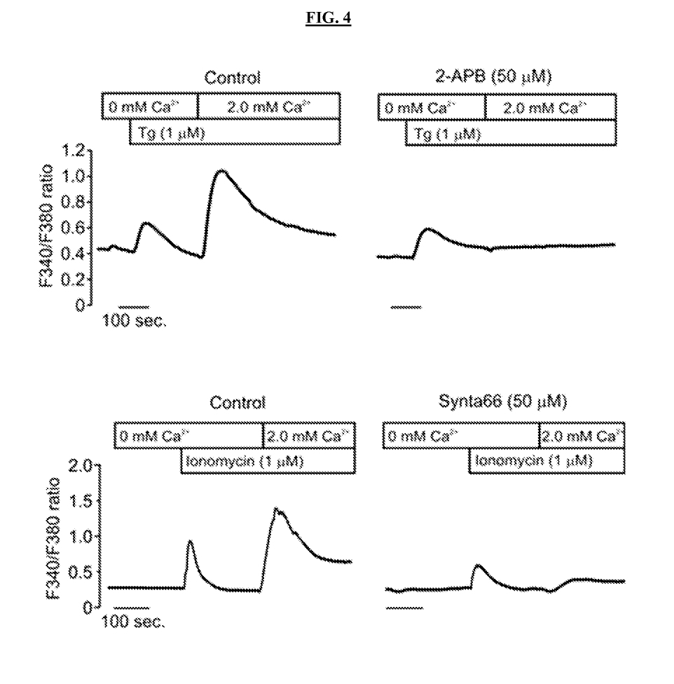

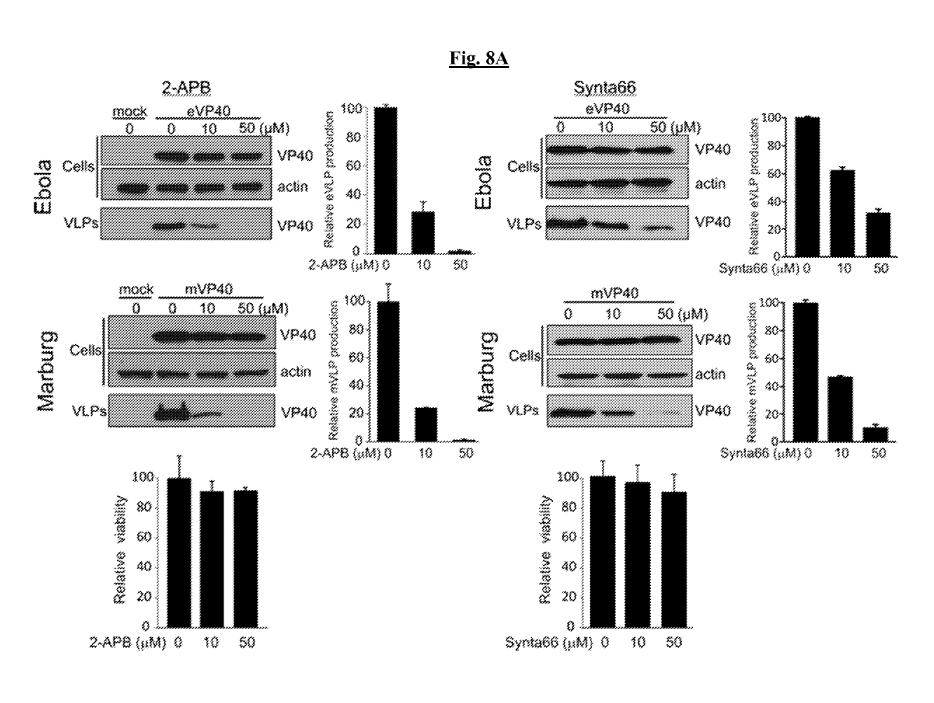

The results from each approach are comparable. For example, in HEK cells that overexpress a dominant negative mutant of Orai1 (E106A), Tg elicited Ca²⁺ release from the ER in Ca²⁺-free solution; however, subsequent superfusion with Ca²⁺-containing medium produced no significant secondary Ca²⁺ rise, confirming a block in calcium permeation of Orai channels (FIG. 4A). Similarly, in STIM1-suppressed cells, Tg released Ca²⁺ from the ER, but no calcium entry occurred following superfusion with Ca²⁺-containing medium. Calcium entry can be rescued in STIM1-suppressed cells by overexpression of STIM1 cDNA. These effects of Orai1 inactivation and STIM1 suppression were similar to the effects on calcium entry of CRAC/Orai channel inhibitors including 2-APB, Synta66 (FIG. 4), and R02959 (FIG. 8B), each of which blocks Ca²⁺ entry following Tg stimulation without affecting its release from the ER. Thus, inactivation or blockade of Orai1 and suppression of STIM1 expression similarly prevent Tg- or ionomycin-mediated Ca²⁺ entry in HEK293 cells and can be used interchangeably to evaluate the role of Orai-mediated calcium entry on steps of enveloped RNA virus budding.

### Example 4: Genetic Inactivation of Orai1 Permeation Blocks eVP40 and mVP40 VLP Production

Expression of the Ebola (e) and Marburg (m) virus matrix protein VP40 in host target cells in the absence of other viral proteins is sufficient to coordinate the production and budding of authentic viral particles. PPxY and PTAP motifs within the Ebola VP40 protein mediate physical and functional interactions between VP40 and a ubiquitin ligase Nedd4 and ESCRT proteins, including Tsg101, and utilizes these host mechanisms to direct virus particle assembly and budding at the plasma membrane. Calcium plays a role in several steps normally involved in ESCRT pathway function, including Nedd4 activation and Alix membrane localization.

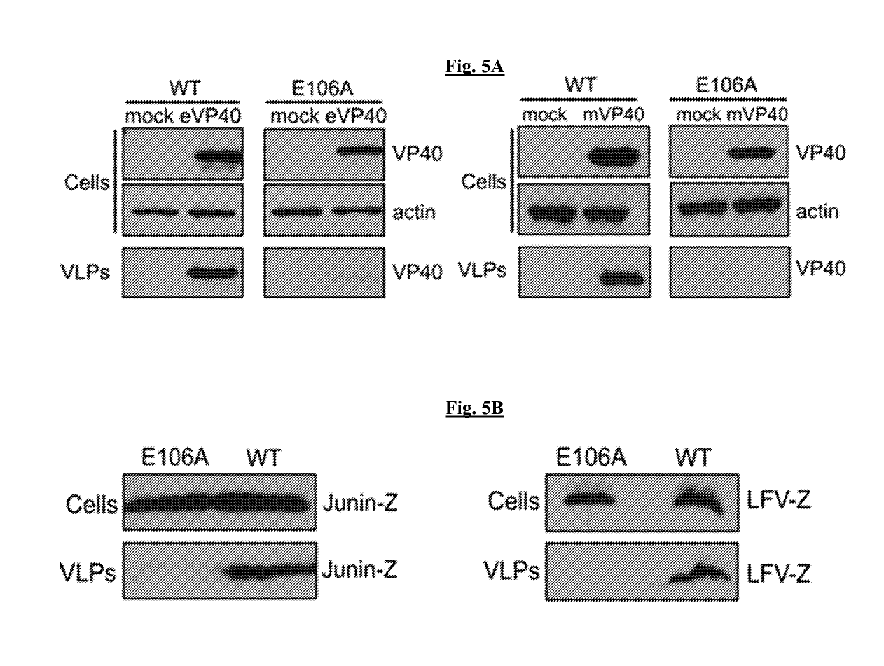

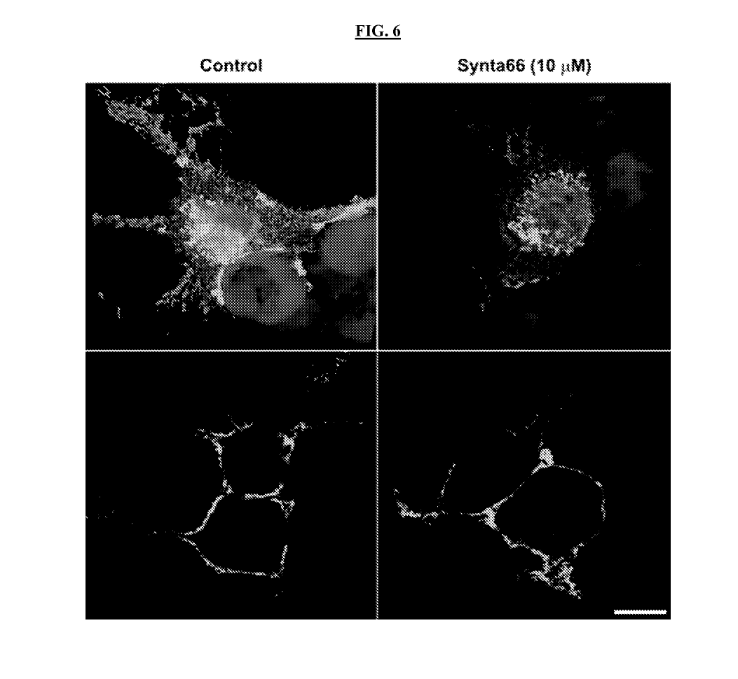

The hypothesis that host calcium signals might regulate late steps of Ebola and Marburg virus budding that rely upon these host mechanisms was investigated. To assess this, eVP40- and mVP40-directed VLP formation was measured in WT HEK293 and in mutant Oral E106A expressing HEK 293 cells. Cellular levels of VP40 and actin were similar in both cell types, indicating no requirement for Orai1-mediated calcium entry in protein synthesis. By contrast, cells expressing permeation defective Orai1 (E106A) were unable to support efficient VLP production, as levels of eVP40 VLPs in the supernatants from these cells were 100-fold lower than those observed in supernatants from WT cells (FIG. 5A). Marburg virus VP40-mediated budding exhibited a similar dependence on Orai1-mediated calcium entry, as VLP production but not cytoplasmic mVP40 levels, was inhibited >50-fold in Orai1 E106A HEK293 cells (FIG. 5B).

Given similar cellular VP40 protein levels in WT and E106A HEK 293 lines, the hypothesis that Ca²⁺ might control a step distal to VP40 synthesis, such as localization to the plasma membrane, was investigated. Visualization of VP40 localization with confocal imaging revealed that the accumulation of VP40 protein at the plasma membrane was comparable in WT and in HEK293 cells that express Orai1 E106A or treated with Synta66 (FIG. 6). However, large filamentous structures containing VP40 that protruded from the surface of untreated or WT cells were not prevelant on cells treated with the Orai inhibitor Synta66. eVP40 Localization was measured using Total Internal Reflectance Microscopy, which is a Z-axis (70 nm) super-resolution technique, also reveals no difference in membrane localization of eVP40 was observed in untreated versus Synta66 treated cells. Together, these results establish a role for Orai1-mediated calcium entry in late steps that regulate the assembly and/or budding of Ebola and Marburg subsequent to VP40 membrane localization.

### Example 5: STIM1 is Required for eVP40 and mVP40 VLP Formation

Given the finding that Ca²⁺ permeation of Orai1 is required for Filovirus VLP formation, the possible requirement of STIM1 for Orai-dependent VLP production was investigated. While STIM1 is the primary physiological trigger for Orai1, STIM2 might play a role in homeostatic calcium signaling as its Kₐ for Ca²⁺ is higher than that of STIM1 and would allow for STIM2 activation at resting (high) ER calcium levels.

Therefore, to determine whether Orai1 control of VLP formation reflects STIM1 activation or possibly constitutive STIM2 activity, VLP formation in STIM1-suppressed HEK293T cells was examined STIM1 siRNA-mediated suppression of STIM1 expression (FIG. 7) had no impact on expression levels of cellular VP40 protein; however, eVP40 VLP formation was significantly diminished by STIM1 siRNA-mediated suppression. Levels of eVP40 VLPs in culture supernatants from STIM1-suppressed cells were >80% lower than levels in cells transfected with random siRNA or empty vector. Furthermore, using a bicistronic vector which suppresses endogenous STIM1 (by targeting the 5′-UTR) and expresses exogenous STIM1 from the human cDNA, STIM1 expression and VLP formation were suppressed and then both rescued to control levels (FIG. 7). Together these results demonstrate that STIM1 is required for both Ebola and Marburg VLP formation and indicate that the canonical pathway of STIM1-mediated Orai1 activation is involved in this process.

### Example 6: Pharmacological Inhibition of Orai with 2-APB, Synta66, and R02959 Blocks eVP40 and mVP40 VLP Budding

The studies using genetic approaches to modulate STIM1 and Orai1 expression or activity demonstrate a requirement for extracellular calcium for VLP egress and establish the STIM1 and Orai1 function in Ebola and Marburg virus VLP formation.

This function of Orai1 was then confirmed pharmacologically. 2-APB (50 μM), Synta66, and R02959 are rapid CRAC channel inhibitors that are active in the 1-50 μM concentration range. 2-APB inhibited eVP40 and mVP40 VLP budding with a dose-dependence that corresponds to its inhibition of Orai-mediated calcium entry and CRAC currents. At a concentration that fully inhibited CRAC channels (50 μM), eVP40 VLPs production was inhibited as much as 60-fold and mVP40 VLP formation by up to 80-fold (FIGS. 8A-8B). In both instances, the suppression was comparable to that observed in Orai1 inactivated and STIM1-suppressed cells. Moreover, neither 2-APB nor Synta66 inhibited cellular accumulation of either eVP40 or mVP40 nor impaired cell viability over the concentration range tested (FIGS. 8A-8B), suggesting the anti-budding effect was due to direct inhibition of Ca²⁺ entry via CRAC channels. R02959 was a more potent inhibitor of Orai1 (˜10-fold) than either 2-APB or Synta66, and at concentrations <5 μM blocked Ca²⁺ entry and eVP40 production by 50-100-fold without any effect on cellular VP40 expression (FIGS. 8A-8B).

### Example 7: Novel 2-APB Related Compound Inhibits eVP40-Mediated VLP Production

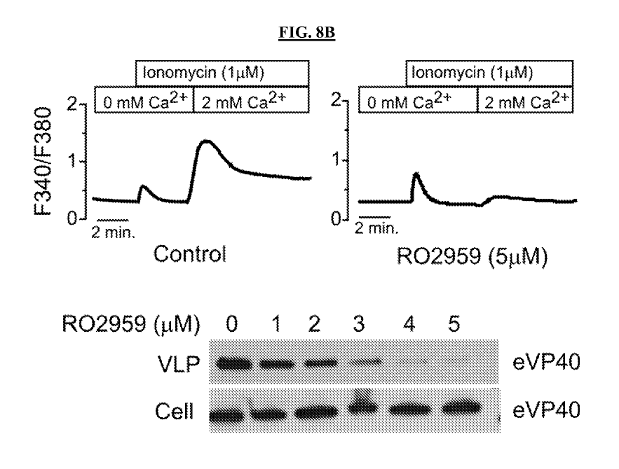

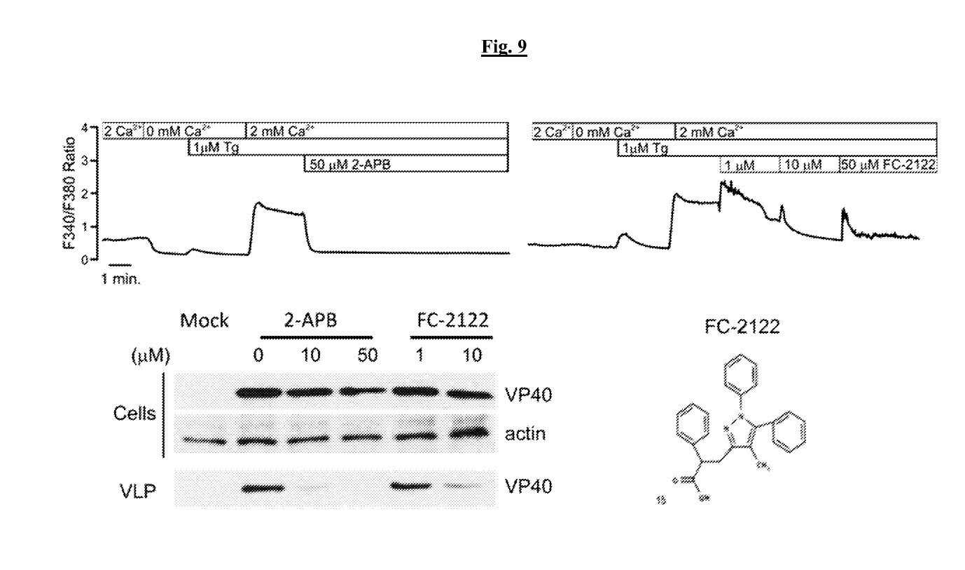

Several structurally related compounds from a proprietary chemical compound library were tested in VLP budding assays and in a high throughput screen for inhibition of Tg-induced calcium entry. Several effective compounds were identified including FC2121 (4-[3-(diphenylmethyl)-1,2,4-oxadiazol-5-yl]piperidineyl]piperidine) and FC2122 (3-(4-methyl-1,5-diphenyl-1H-pyrazol-3-yl)-2-phenylpropanoic acid), which exerted partial dose-dependent inhibition of Tg-induced calcium entry and a corresponding inhibition of VLP formation (FC2122, FIG. 9). In the case of FC2122, cellular eVP40 expression at 1 μM and 10 μM were not different from mock treated cells, yet VLP VP40 levels were decreased to a comparable extent as by 2-APB. FC2122 inhibited VP40 expression at concentrations above 10 μM but exerted mild suppression of actin and cellular VP40 expression. Importantly, more than 30 structurally related compounds that exerted no inhibition of calcium signaling also had no effect upon eVP40-mediated VLP formation. These studies with CRAC channel inhibitors further support a critical role for calcium entry via Orai1/CRAC channels in VLP formation.

### Example 8: Orai1-Mediated Regulation of Live BSL-2 Virus Budding

The pathogenesis of Junin and Lassa Fever (LF) viruses is similar to Ebola and Marburg viruses, all being NIAID Category A bioterror agents that produce hemorrhagic fevers and death. Like Ebola and Marburg viruses, a matrix (Z) protein of Junin and Lassa Fever viruses orchestrates VLP assembly and egress of Therefore, the role of Orai-mediated calcium signaling on Junin and LF Z protein-mediated VLP formation was investigated in WT 293T and E106A cells. 48 hours post-transfection, both Junin and Lassa Fever Z-mediated VLP production from HEK293T E106A cells was substantially lower (>20-fold) than from WT 293T cells (FIGS. 5A-5B). While matrix protein-mediated VLP formation is an authentic and robust model for studying the regulation of virus budding, VLP findings were further validated by examining the effect of Synta66 on production of live enveloped RNA viruses.

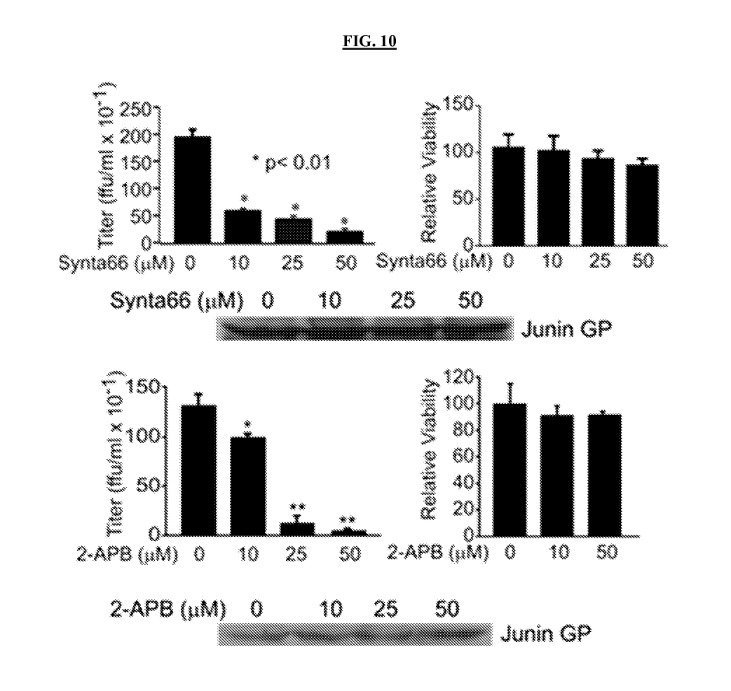

As there is a live attenuated vaccine strain of Junin virus (Candid-1 strain; C1) that can be handled under BSL-2 conditions, a focus forming assay was used to detect JUNV Cl replication (Cuevas, et al., 2011, J. Virol. 85:11058-11068; Lu, et al., 2014, J. Virol. 88:4736-4743). Briefly, Junin virus foci (clusters or virus infected cells) were observed and quantified by indirect immunofluorescence using anti-Junin GP antiserum (FIG. 10). Enumeration of foci in 10 fields for each sample revealed a statistically significant, Synta66 dose-dependent reduction in the number of Junin foci (FIG. 10). No effect was observed on cellular expression of JUNV glycoprotein (GP) or actin in infected cell extracts (FIG. 10) or on viability of VeroE6 cells (FIG. 10) under conditions mimicking those used for infection experiments (FIG. 10) with no effect on cellular levels of Z protein or action. Similar results were observed using 2-APB, which induced 10-fold decrease in the number of foci formed by virions released from cells (FIG. 10). Together, these results correlate well with data obtained from Z VLP budding assays and demonstrate that inhibition of Orai in Vero cells by Synta66 and 2-APB significantly reduces the budding efficiency of live Junin Candid-1 virus.

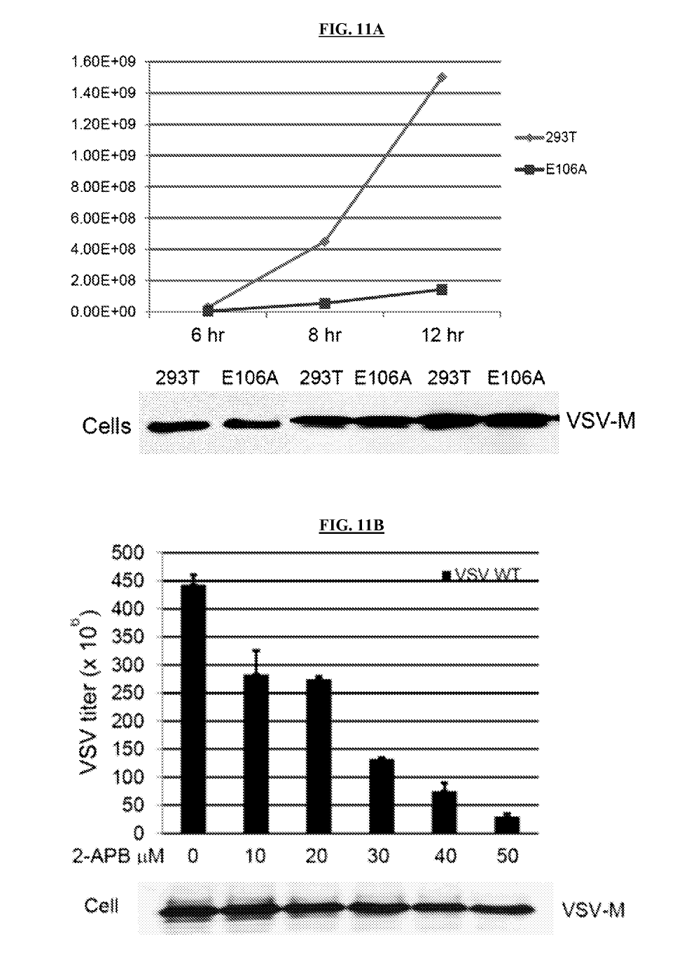

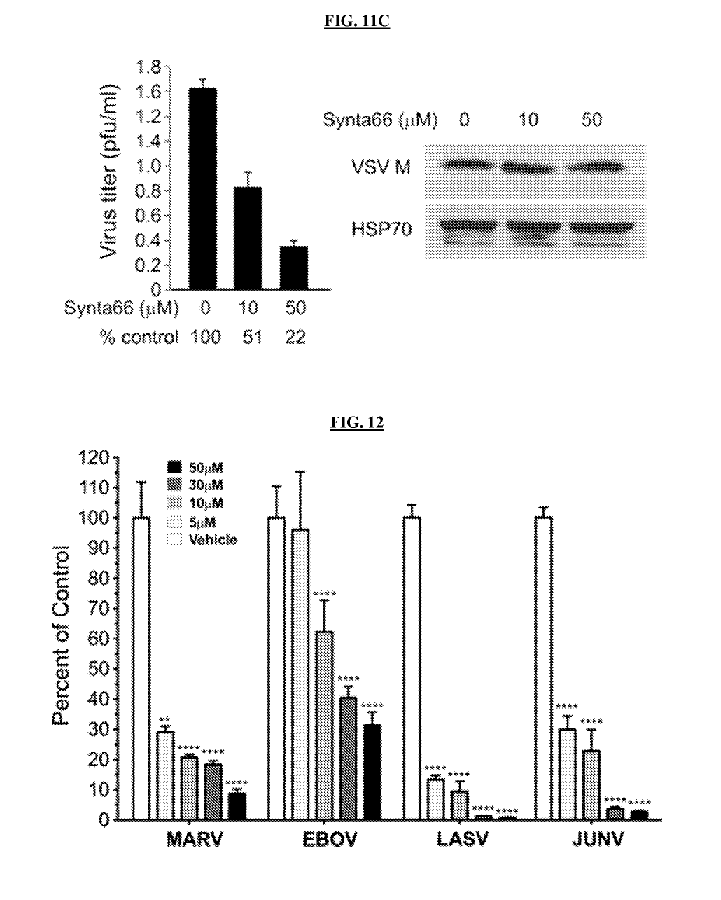

VSV, a rhabdovirus, is another BSL-2 surrogate of Ebola virus, and, like Ebola, Marburg and HIV-1, is an enveloped, negative-sense RNA virus, which utilizes L-domain interactions with host ESCRT proteins for budding. Therefore, the hypothesis that calcium entry via CRAC channels regulates VSV budding from host cells was investigated. In these experiments, wild type HEK293T and E106A HEK293 cells were infected with VSV at an MOI of 0.1, and supernatant samples were harvested at 6, 8, and 12 hours post-infection. Virions budding into supernatants of infected cells were quantified using a standard plaque assay performed in triplicate on BHK-21 cells. Consistent with VLP results, VSV titers from E106A cells were reduced by approximately 10-fold from those of WT HEK293T cells, while viral protein expression in both WT and E106A cells was identical (FIG. 11A). Moreover, 2-APB inhibited live VSV viral particle budding with a dose dependence that parallels the sensitivity of Orai1 to 2-APB inhibition (FIG. 11B) with only a slight effect on cellular M protein expression. Synta66 also produced a dose-dependent inhibition of VSV budding (FIG. 11C). These data demonstrate that Orai1 regulates live VSV budding to a comparable extent as Ebola and Marburg VP40. Together, these results establish a general requirement for Orai1-mediated calcium entry in late steps of enveloped RNA virus (filovirus, arenavirus, rhabdovirus) VLP egress.

### Example 9: Pharmacological Inhibition of Orai Channel Activity Blocks Budding of Live BSL-4 Pathogens, EBOV, MARV, LASV, and JUNV

Based on the sensitivity of VLP production and live JUNV (Candid-1) and VSV to Orai inhibitors, the hypothesis that pharmacological inhibition of Orai1 blocks budding and spread of live pathogenic strains of EBOV, MARV, LASV, and JUNV was investigated. For these experiments, the effect of Synta66 on viral transmission in a cell culture model of infection was investigated using HeLa cells (FIG. 12) or HEK293T cells. Cells were infected with live pathogenic LASV, JUNV, MARV or EBOV at a multiplicity of infection=0.1. One hour post infection, cells were treated with vehicle alone, or Synta66 at the indicated concentrations. 72 hrs (LASV, JUNV) or 96 hrs (MARV, EBOV) post infection, cells were fixed and stained with virus specific antibodies to quantify the impact of Orai inhibition on infection.

For each virus (LASV, JUNV, MARV, and EBOV) a steep dose-dependent inhibition by Synta66 of budding and spread was observed. Interestingly, Synta66 exerted more robust inhibition of arenavirus than filovirus budding, consistent with effects of Orai blockers on eVP40 and mVP40 VLP production (FIG. 8A-8B). Viability assays demonstrated these effects are not due to toxicity. Together, the results prove that Orai1-, STIM1-, and Ca²⁺-regulated steps of virus budding represent novel and viable targets for broad-spectrum therapeutic inhibition of budding and spread of a range of BSL-4 pathogens.

### Example 10: Orai1 Regulation of HIV-1 Gag-Mediated VLP Formation

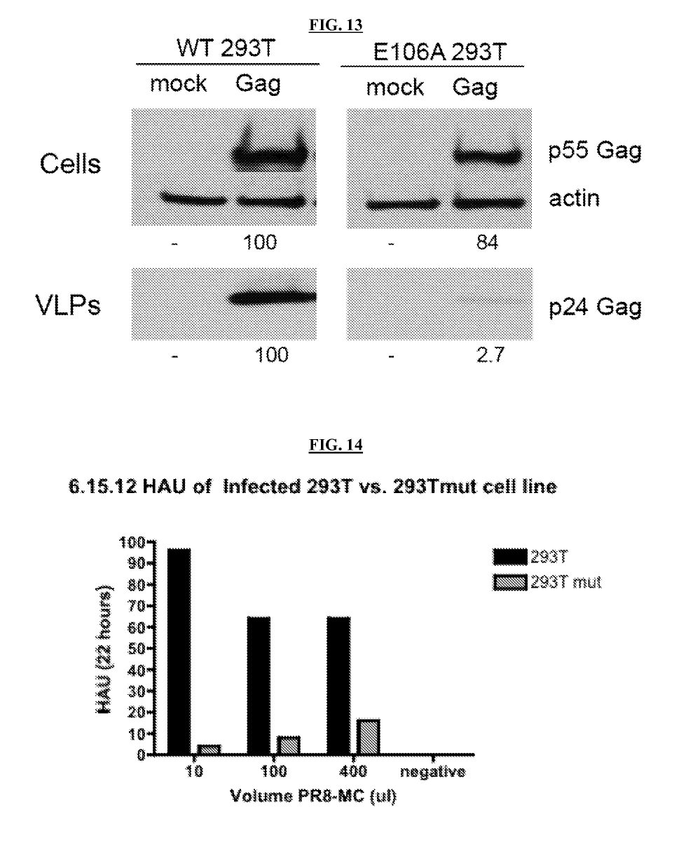

One common feature of both Ebola and Marburg virus VP40-mediated VLP assembly and budding is the involvement of L (late)-domain-mediated interactions with ESCRT family proteins. A number of L-domain interacting proteins are candidates for regulation by calcium, including Nedd4 and Tsg101. Given the apparent universal role of the host ESCRT pathway in assembly and budding for most RNA viruses and the regulation of ESCRT functions by calcium, the hypothesis that calcium entry via Orai1 might play a general role in L-domain-mediated budding of enveloped RNA viruses was investigated. The initial focus was initially placed on HIV-1 Gag, at least because Gag L-domain interactions with Nedd4 are required for its efficient budding. The role of Orai in HIV-1 Gag-mediated VLP formation was tested using the approach developed to evaluate Ebola and Marburg virus VLP formation. HIV-1 Gag is the functional homolog of Ebola and Marburg VP40, and interacts with host ESCRT proteins via L-domains to promote efficient egress of HIV-1 Gag VLPs. Like Ebola and Marburg VP40, expression of Gag is sufficient to drive VLP production. However, in HEK293 lines that stably overexpress the dominant negative Orai1 E106A mutant, Gag-mediated VLP production was substantially reduced without any decrease in cytoplasmic Gag expression (FIG. 13). These results point to a similar requirement for Orai1-mediated calcium signaling in HIV-1 virus budding and further establish this as a general mechanism for regulating enveloped RNA virus budding.

### Example 11: Orai1 Regulation of Influenza Virus Budding

The present findings with Ebola, Marburg, HIV-1, Junin, Lassa fever and Vesicular Stomatitis Virus suggest that Orai1-mediated calcium entry is a common requirement for efficient budding of enveloped RNA viruses. Therefore, the role of Orai1 in Influenza A virus budding, because its pathogenesis and ease of transmission have profound public health importance and impact, was also examined Although vaccines are formulated to anticipate seasonal variants, influenza virus (like most RNA viruses) evolves rapidly via mutation. Thus, anti-influenza drugs (e.g., relenza and tamiflu) and vaccines may be available, but high rates of mutation confer resistance to and diminished efficacy of these antivirals. As neither vaccines nor drugs for influenza viruses produce long lasting or broad-spectrum efficacy, there is a critical need for broad spectrum host-oriented therapeutics that target immutable and essential host proteins required for Influenza budding and transmission.

While influenza virus assembly and budding may be more complex than that of Ebola, Marburg and HIV-1 (in that expression of a single (matrix) protein is not sufficient for VLP formation), the role of Orai1 in budding of live Influenza was tested. Live viral titers (as measured by HA units) were significantly lower in supernatants harvested from cells expressing defective Orai1 E106A channels (labeled 293mut) at low, medium and high levels of infection (FIG. 14). Interestingly, the amount of virus on the cell surface was the same in both cell types, consistent with the idea that Orai1 specifically regulates virus budding. Thus, Influenza A represents yet another enveloped RNA virus that requires Orai1-mediated calcium entry for efficient budding.

### Example 12: Mechanisms by which Ca²⁺ Regulates Enveloped RNA Virus Assembly and/or Budding

Among the potential calcium targets that are involved in late steps of enveloped RNA virus budding are several members of the Endosome Sorting Complex Required for Transport (ESCRT) pathway. The ESCRT complex is normally involved in protein sorting/recycling and in cytokinesis by mediating membrane bending and scission. This process is topologically identical to that observed during virus budding from the plasma membrane. Indeed, host ESCRT proteins promote efficient virion assembly and budding at the plasma membrane. Specifically, interactions between L-domains of virus matrix proteins and components of ESCRT regulate late stages of budding for a number of enveloped viruses including Marburg, Ebola, HIV-1, and VSV. Ebola VP40 has two overlapping L-domain motifs between amino acid positions 7 and 13: ₇PTAPPEY₁₃. The PTAP and PPEY L domains interact with the host proteins Tsg101 and Nedd4, respectively, and in certain embodiments, VP40 can facilitate the formation of budding virions by co-opting the function of these host ESCRT proteins. Nedd4 functions as an E3 ubiquitin ligase, and, importantly, it can be activated by Ca²⁺ binding to C2 domains, which trigger the release of its ligase activity from auto-inhibition. Subsequent mono-ubiquitilation of VP40 by Nedd4 is critical for the subsequent recruitment of other ESCRT factors, including Tsg101. Other potential calcium-dependent steps in viral budding downstream of Nedd4 and Tsg101 include the calcium-dependent activation of an endosomal (calcium-permeant) channel called TRPML1 by a Tsg101 associated ESCRT protein ALG-2.

Regulation of eVP40 Budding by an Alternative Ca²⁺-Permeant Channel: TRPML1.

TRPML1 is expressed in lysosomes and plays a role in focal exocytosis, phagosome biogenesis, and specifically in vesicle scission. Interestingly, TRPML1 overexpression in HEK293 triggers the formation of filamentous projections on the plasma membrane that are indistinguishable from eVP40-mediated VLP projections generated during budding. The hypothesis that TRPML1 regulates eVP40-mediated VLP formation was investigated.

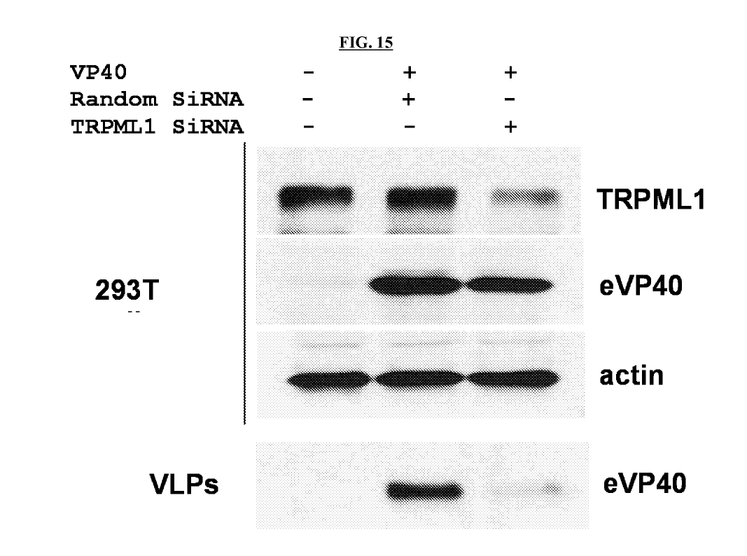

TRPML1 suppression significantly decreases eVP40-mediated VLP production in HEK293 cells without any impact on cellular VP40 expression (FIG. 14). TRPML1 has a role in vesicle scission, and it is regulated in a calcium-dependent manner by the Alix/ALG-2 complex and PI(3,5)P2. PI(3,5)P2 is a rare phospholipid found principally in endosomes, but also in the plasma membrane, that has been implicated in the control of TRPML1 function. Like TRPML1, PI(3,5)P2 regulates retrograde trafficking from the vacuole/lysosome to the late endosome/MVB, and this reflects its control of TRPML1 localization and activation. The PI(3,5)P2 precursor PI(3)P is generated from PI by Vps34, a class III PI3kinase which activity is regulated in a Ca²⁺-dependent manner by Calmodulin. PI(3)P is then converted to PI(3,5)P2 by PIKfyve kinase (FIG. 18). As an indication of this requirement for PI(3,5)P2 in control of TRPML1 function, individuals with defects in PIKfyve activity exhibit lysosomal trafficking abnormalities that phenocopy those associated with TRPML1 defects. Moreover, the PIKfyve inhibitor YM201636, which blocks PI(3,5)P2 production and disrupts endomembrane transport, also inhibited retrovirus budding. TRPML1 activation may represent a distal and or the ultimate, calcium regulated step in enveloped RNA virus budding. In this regard, inhibition of PIKfyve, the enzyme that generates PI(3,5)P2, also significantly inhibited eVP40 VLP production. Experiments in which TRPML1 was suppressed in HEK293 cells demonstrated the critical role TRPML1 plays in filovirus VLP formation (FIG. 15).

### Example 13: Ca²⁺-Dependent Regulation of Alix in Filovirus Budding and TRPML1 Activation

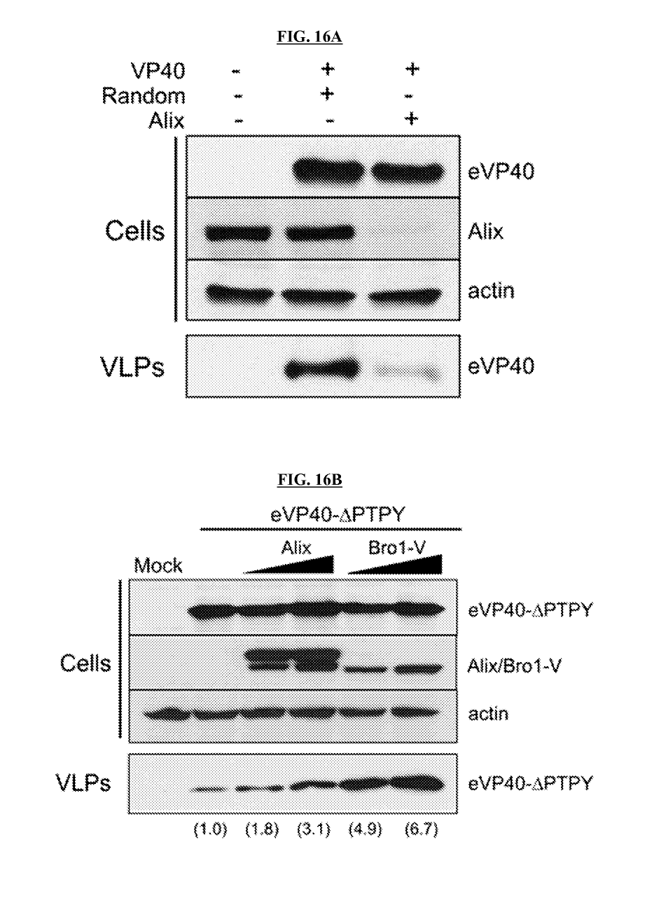

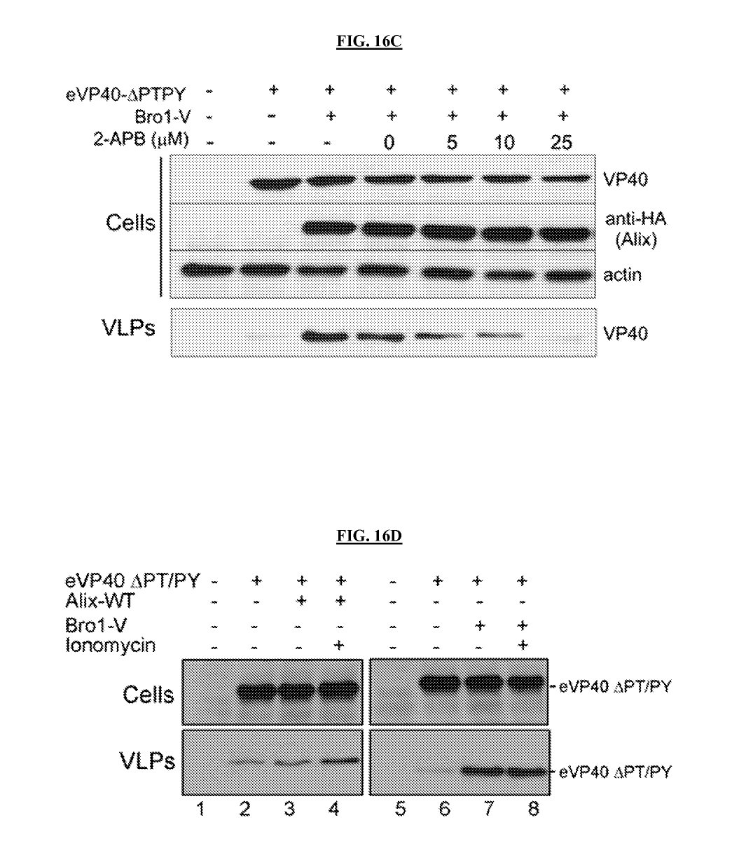

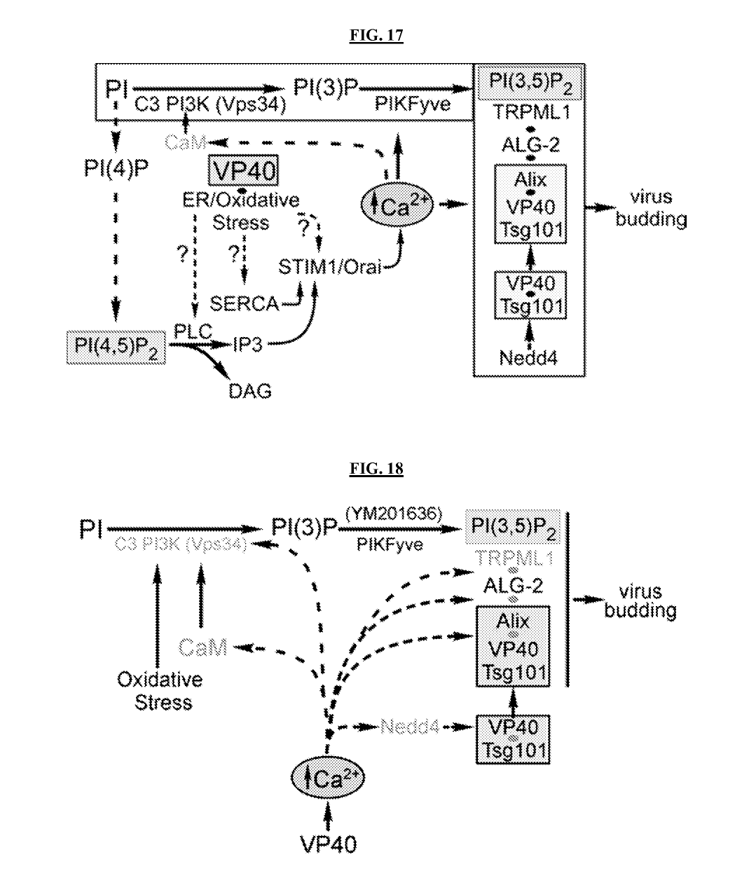

While HIV-1 Gag-mediated VLP formation involves Alix binding to its YpxL L-domain motif, a similar interaction and function for Alix in VP40-mediated VLP formation has not previously been identified. As demonstrated herein, Alix plays a role in VP40-mediated VLP production (FIG. 16A); moreover, a truncated variant of Alix can rescue defective VLP production from a double L-domain mutant of VP40 (VP40-APT/PY mutant with PTAP and PPxY motifs deleted), which cannot interact with Tsg101 or Nedd4 (FIG. 16B). Interestingly, a truncated Alix Bro1-V fragment (minus PRD) efficiently rescued VLP formation more efficiently than full length Alix. This rescue involved Alix binding to VP40 via a novel Ypx(n)L-type L domain in VP40 spanning amino acids 18-26.

Indeed, Ca²⁺ plays at least two roles in Alix-dependent VLP formation. The first mechanism is indicated by the finding that Alix Bro1-V rescue of VLP production is Orai1-dependent (2-APB sensitive, FIG. 16C). Thus, while Ca²⁺ controls a step distal to VP40:Alix binding, it also promotes the rescue of defective eVP40-APT/PY-mediated VLP formation by full length Alix. Moreover, while eVP40-APT/PY-mediated VLP formation is rescued less efficiently by full length Alix, mobilization of Ca2+ augmented the ability of full length Alix to rescue VLP production (FIG. 16D). Together, these results suggest that the Alix PRD stabilizes a folded, inactive conformation, that masks the Bro1 and V domains that are critical for binding VP40 (via the YPxL motif) to facilitate budding and that Alix represents yet another target for Ca²⁺ regulated control of enveloped RNA virus budding.

### Example 14: Mechanism of VP40-Mediated Orai1 Activation

### Hypothesis: Filovirus VP40-Mediated STIM1 Activation Triggered by IP3R.

The initial study comprises determining whether Filovirus VP40 activates STIM1 via the classic mechanisms involving IP3-dependent depletion of Ca²⁺ from the ER. To explore the role of IP3R in VP40-mediated Ca²⁺ mobilization (and EBOV VLP formation), first examine the requirement for phospholipase C (PLC), a family of enzymes that cleave IP3 (and DAG) from membrane PI(4,5)P₂ typically following ligand/receptor activation, is investigated.

A role for PLC in VP40-mediated VLP formation and Ca²⁺ signaling is examined using the PLC selective inhibitor U73122 (1-[6-[[(17β)-3-Methoxyestra-1,3,5(10)-trien-17-yl]amino]hexyl]-1H-pyrrole-2,5-dione). An inactive analog of this inhibitor (U73433) serves as a negative control for these experiments. If U73122 inhibits VLP formation, then ones determines whether this reflects Ca²⁺ release from the ER (with the ER Ca²⁺ reporter D1ER) due to elevated cellular IP3 during the initiation and sustained phases of VP40-mediated VLP formation.

Given that VP40 expression and the VP40-mediated Ca²⁺ signals develop gradually over 24 hours, it may be impossible to detect a measurable spike in IP3 concentration. If this is the case, the membrane permeant IP3R antagonist heparin is used to infer a role of IP3R in VP40-mediated Ca²⁺ signals and VLP formation. If heparin blocks signaling and/or VLP production, then siRNAs are used to suppress each IP3R isoform (1-3) to determine which is responsible. If IP3R are not found to be involved in VP40 mediated Ca²⁺ signaling, then other mechanisms are addressed as outlined elsewhere herein. If IP3R suppression and/or heparin experiments implicate IP3R in Ca²⁺ signaling and VLP formation, in the absence of PLC activation, then it is possible that VP40 activates IP3Rs via a direct physical coupling mechanism. Biochemical methods (co-precipitation) or FLIM, as demonstrated for VP40 and Tsg101 (FIGS. 19A-19B), are used to visualize the dynamic interactions between overexpressed IP3R-CFP and VP40-GFP to assess whether these interactions might trigger Ca²⁺ release from the ER (measured as an increase in cytoplasmic Ca²⁺ levels with the genetically encoded Ca²⁺ indicator R-GECO1; FIG. 3).

### Hypothesis: STIM1 Activated by VP40-Induced Production of Reactive Oxygen Species.

While the classical mechanism of STIM1 activation involves Ca²⁺ release from the ER, post-translational modifications of the STIM1 N-terminus can mimic the effects of Ca²⁺ dissociation to induce STIM1 activation without any change in ER calcium levels. This Ca²⁺-independent STIM1 activation is caused by S-glutathionylation of STIM1 at cysteine residue 56. This S-glutathionylation induces a C-terminal conformational change in STIM1 comparable to that induced by Ca²⁺ dissociation and induces its oligomerization and consequent activation of Oral. As ER/oxidative stress are general consequences of viral infection and excess viral protein synthesis, such stress could directly induce ROS-dependent STIM1 activation.

To examine the role of ROS production in VP40-mediated STIM1 and Orai1-dependent Ca²⁺ signaling and VLP formation, ROS inhibitors (e.g. diphenyliodonium (DPI), N-acetylcysteine (NAC), and the like) are used. The effect of these inhibitors on VP40 mediated Ca²⁺ signals is examined. These measurements are performed on cells that express the genetically encoded Ca²⁺ indicator R-GECO-144 (FIG. 3). STIM1 Glutathionylation in STIM1 immunoprecipitates from VP40-transfected and control vector transfected HEK293T cells are also analyzed using an anti-GSH antibody. If ROS inhibitors block the VP40 mediated Ca²⁺ signal, they may also block STIM1 membrane localization. To address this, STIM1-mCherry localization is concurrently monitored in these experiments and complementary experiments are performed in STIM1 suppressed cells rescued with a C56A STIM1-mCherry mutant that cannot be glutathionylated. Overexpression of VP40, or any protein for that matter, may induce ROS regardless of its role in STIM1 activation. Therefore, to assess the extent to which ROS production is the primary mechanism of STIM1 activation and viral budding in vivo during normal viral infection, the effect of membrane permeant ROS inhibitors is tested on the production of live virus.

### Hypothesis: Modulation of SERCA Expression or Activity Control VP40-Mediated STIM1 Activation.

A decrease in ER Sarco-endoplasmic reticulum Ca²⁺ ATPase (SERCA) expression or activity that establishes a new ER equilibrium Ca²⁺ concentration below that required for STIM1 activation (˜400 μM) may initiate STIM1 activation independently of IP3-mediated Ca²⁺ release. Therefore the hypothesis that VP40 induces a decrease in SERCA expression in VP40 versus vector transfected cells in lysates of HEK293T and HeLa cells infected with live Ebola and Marburg virus is investigated. SERCA pump activity in vector vs. VP40 expressing cells is measured to determine whether VP40 dynamically modulates SERCA activity. To do this, the ATP sensitive rate of Ca²⁺ flux into the ER is determined (Neumann, et al., 2005, Proc. Natl. Acad. Sci. USA 102:17071-17076). ROS can also modulate the activity of SERCA. For example, T lymphocytes compensate for low oxygen (hypoxia) by increasing SERCA2 expression and activity to enhance Ca²⁺ uptake into the ER. While it is unknown if increased oxygen radicals have the opposite effect, if a change in activation is observed, then this mechanism will be addressed with ROS inhibitors.

### Identification of Mechanism of Sustained VP40-Mediated Ca²⁺ Signaling.

While there is a critical requirement for Orai1-mediated Ca²⁺ entry in Ebola and Marburg virus budding, Orai1 typically exhibits fairly rapid time and Ca²⁺-dependent inactivation. The fact that VP40 induces a gradual and protracted increase in Ca²⁺ concentration suggests that additional mechanisms participate in this sustained response.

The hypothesis that ER Ca²⁺ levels are depleted by VP40 and then remain depleted during the extended time course (24 hours) of VP40 expression and Ca²⁺ signaling is investigated. To obtain quantitative measurements of ER Ca²⁺, the genetically encoded ER Ca²⁺ reporter (D1ER) is expressed in HEK293T cells. Control measurements are performed on cells expressing the vector backbone. It is also possible that STIM1 is initially activated by Ca²⁺ depletion, but that its persistent activation is Ca²⁺-independent. Therefore, if ER do not remain depleted for the duration of the experiment, the duration of STIM1 activation is quantified by measuring the extent and time course of mCherry STIM1 plasma membrane localization using TIRF microscopy. If STIM1 membrane localization persists throughout the duration of VP40-induced Ca²⁺ signaling while ER Ca²⁺ stores are filled, it is determined whether ROS regulates this persistent membrane localization. In this scenario, it is assessed whether Orai1 remains open throughout the timecourse of signaling by perfusing cells with Ca²⁺-free medium or by blocking Orai with inhibitors (e.g., Synta66 or 2-APB) at different times between 0 and 24 hours. A resulting decrease in cytoplasmic Ca²⁺ concentration indicates that Orai1 remains open. If, however, STIM1 does not persist at the plasma membrane and Orai does not remain open throughout the duration of VP40-mediated Ca²⁺ signaling, this suggests that VP40 sustains Ca²⁺ elevation by either inhibiting SERCA-dependent Ca²⁺ re-uptake into the ER or extrusion from the cell by PMCA pumps. To address this latter possibility, the regulation of PMCA expression by VP40 and live virus is measured, and the PMCA activity is also measured throughout the timecourse of VP40 expression.

### Example 15: Role of VP40 L-Domains in STIM1 Activation

The mechanisms by which VP40 interactions initiate cellular Ca²⁺ signals are unexplored and their identification may provide insight into novel mechanisms that control viral budding, thereby revealing potential targets for regulating this budding. Studies from yeast point to a potential direct link between VP40 and Ca²⁺ signaling, as SERCA expression is regulated by the yeast Tsg101 homolog Vps23.

Given the L-domain (PTAP) dependence of Tsg101 binding to VP40 and the critical role identified for Tsg101 in VP40 mediated VLP formation, the influence of VP40 PTAP L-domain interaction with Tsg101 on VP40-mediated Ca²⁺ signals is investigated. For this, a PTAP deletion mutant of VP40 that prevents the known interaction between VP40 and Tsg1019 is utilized. Alternatively, the role of Tsg101 in VP40-induced Ca²⁺ signals is more generally examined by suppressing Tsg101 expression. In either case, if inhibition of Ca²⁺ signaling is observed, the hypothesis that Tsg101 regulates SERCA expression or activity is investigated using the VP40 PTAP mutant and/or Tsg101 suppressed cells. Pending the outcome of these studies, the role of PPxY and the novel YPx(n)L L-domains by which VP40 interacts with Nedd4 and Alix, respectively, is examined.

As Ebola and Marburg VP40 orchestrate VLP production by a similar mechanism as the HIV-1 matrix protein Gag, it is possible that the PLC inhibitor U73122 similarly suppresses VP40-mediated Ca²⁺ signals and VLP formation by preventing IP3 production. However, an alternative interpretation may be based on the fact that direct interactions between matrix protein (Gag in this case) and the PLC substrate PI(4,5)P₂ regulate VLP assembly at the plasma membrane. This is critical in light of the known mechanism of U73122 action, which does not inhibit PLC activity, but rather reduces availability of the PLC substrate PI(4,5)P₂. Thus, the interpretation of the effects observed with U73122 is based upon complementary experiments that measure IP3 levels under control conditions; the inhibitor may decrease VLP formation simply by decrease PI(4,5)P₂ availability and VP40 binding to the plasma membrane. If IP3 is indeed generated, it may also be expected that steady state IP3 levels decrease relatively quickly and not remain elevated throughout the duration of signaling and budding. Thus, the persistent Ca²⁺ signals may reflect an ER Ca²⁺-independent mechanism (e.g., inhibition of SERCA activity) or ER Ca²⁺-independent STIM1 (and Orai1) activation. If IP3 levels decay yet ER Ca²⁺ levels remain low, this suggests that SERCA expression or activity is decreased or that oxidative stress may be promoting persistent STIM1 activation.

Without wishing to be limited by any theory, STIM1 and Orai1 may not remain “activated” throughout the duration of Ca²⁺ signaling because (1) it may be energetically and biologically inefficient, (2) elevated Ca²⁺ may activate SERCA and PMCA pump activity to remove Ca²⁺ from the cytoplasm and refill stores, and (3) Orai1 exhibits Ca²⁺-dependent inactivation that normally preclude persistent cytoplasmic Ca²⁺. However, if TIRF measurements reveal persistent STIM1 membrane localization (suggesting persistent Orai1 activation) and this is confirmed by Orai1 inhibitors, then measurements of ER Ca²⁺ may indicate whether this activation is due to persistent ER Ca²⁺ depletion or whether STIM1 plasma membrane localization is uncoupled from ER Ca²⁺ levels. In this instance, the use of ROS inhibitors may reveal whether this reflects ER Ca²⁺-independent ROS-induced STIM1 activation. Alternatively, If STIM1 does not remain localized to the ER yet cytoplasmic Ca²⁺ levels remain elevated, it may suggest that PMCA expression or activity is diminished and inhibition of Ca²⁺ extrusion from the cell accounts for persistently elevated Ca²⁺. It is theoretically possible that the ER is refilled, that STIM1 is not localized to the plasma membrane, but that Orai remains open. In this instance, the possibility that VP40 directly activates Orai1 or blocks its inactivation may be explored. In this instance, upon removal of extracellular Ca²⁺ or application of Orai1 blockers, cytoplasmic Ca²⁺ levels may decrease, whereas sustained cytoplasmic Ca²⁺ levels driven by inhibition of PMCA (and SERCA) activity may not be affected by removal of extracellular Ca²⁺. As only a few of these proposed mechanisms are mutually exclusive; it may be that the time dependent change in cytoplasmic Ca²⁺ concentration reflects multiple levels of regulation. Thus, the key objective of these studies is to determine which of these mechanisms is dominant during viral budding, as the proteins involved represent novel and effective therapeutic targets.

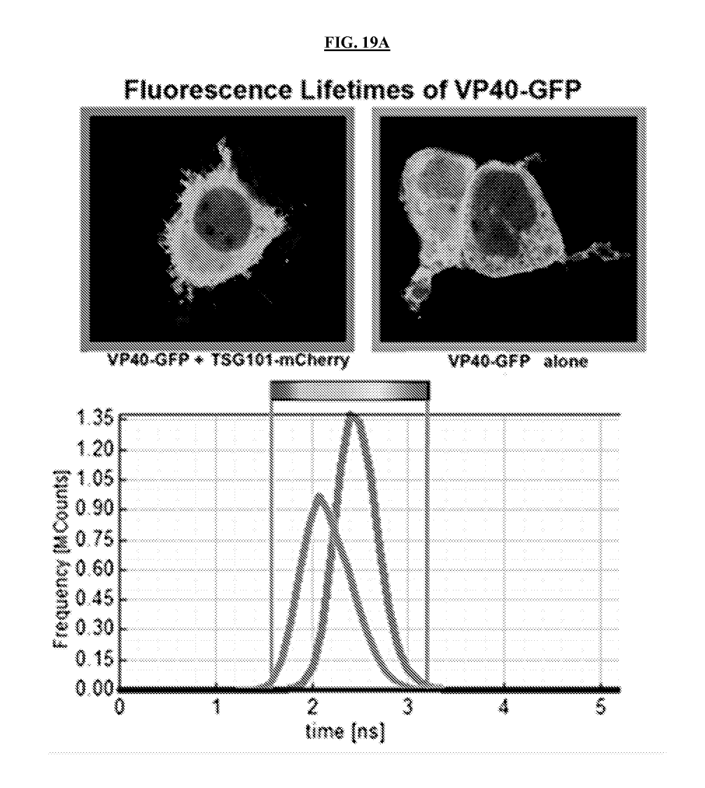

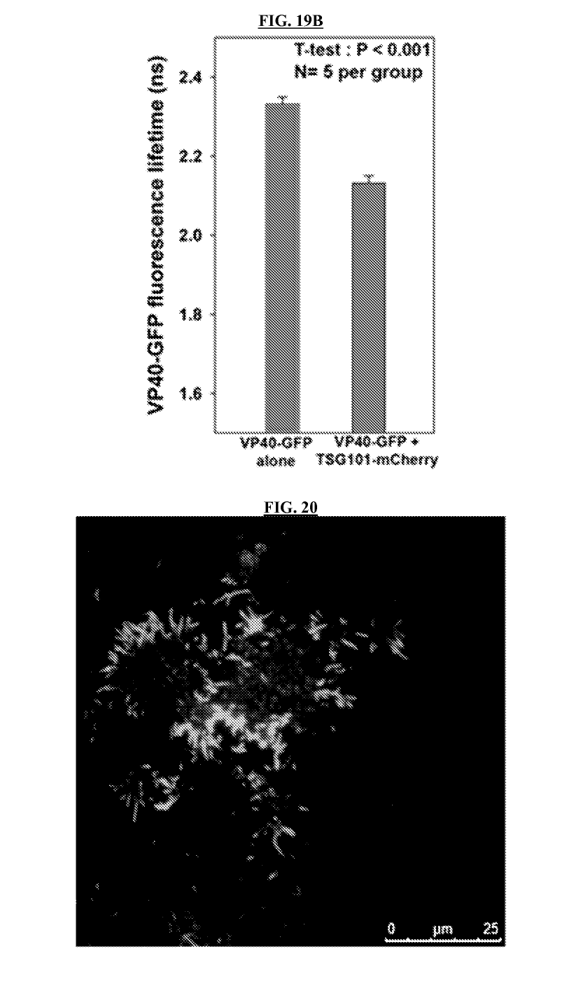

Historically, measuring Ca²⁺ for extended periods has been challenging because standard organic cytoplasmic Ca²⁺ dyes (Fura-2 and Fluo-4) leak from cells and are highly sensitive to photobleaching, and because it is challenging to maintain cells in a viable state for these measurements. To circumvent these limitations, the genetically encoded Ca²⁺ indicator R-GECO-1, which has proved to be highly photostable and retained indefinitely in cells, is used. The microscope is enclosed in a custom made temperature, CO₂, and humidity controlled environmental chamber. These innovations allows for quantifying Ca²⁺ in cells under stable physiological conditions for extended time periods (hours to days). Although global changes in cytosolic Ca²⁺ concentration are assessed, Ca²⁺ signals may be highly localized to sites where VP40 orchestrates virus assembly (i.e., subplasmalemmal domains). If indicated, TIRF (FIG. 20) imaging may be used to measure highly localized cytoplasmic Ca²⁺ signals at sites of VLP assembly and scission, and the mechanisms by which these localized signals are generated, including a potential role for TRPML1.

### Example 16: Mechanism by which Orai1-Mediated Ca²⁺ Entry Regulates Filovirus Budding

As demonstrated herein, genetic inactivation of Orai1 (FIGS. 5A-5B), STIM1 suppression (FIG. 7), and selective Orai1 blockers (FIGS. 8A-8B) each profoundly inhibits VP40-mediated VLP formation. Furthermore, Orai1 regulates the transmission of live Ebola and Marburg viruses (FIGS. 11A-11C). Together, these studies establish that Orai1-mediated Ca²⁺ entry is critical for Filovirus VLP assembly and/or budding; however, the mechanisms by which Ca²⁺ does so are completely unexplored. One hypothesis is that Ca²⁺-dependent control of Alix activation and interactions with ALG-2 regulates the localization and activation of TRPML1, an endosomal cation channel to the plasma membrane. Further, VP-40-induced Ca²⁺ signaling may regulate the generation of PI(3,5)P₂, a rare phospholipid agonist for TRPML1 and that together the Ca²⁺ dependent localization and activation of TRPML1 may generate plasma membrane TVS-like structures from which viral particles bud (FIG. 20). Consistent with such a mechanism, studies visualizing VP40 localization on the surface of HEK293 cells demonstrates that inhibition of Ca²⁺ entry with the Orai inhibitor Synta66 inhibits the formation of VP40-induced membrane protrusions (FIG. 6).

Hypothesis: Ca²⁺ Regulate VP40 Interactions with Tsg101 by Activating Nedd4.

Nedd4 is an E3 ubiquitin ligase which activity is regulated by Ca²⁺ through its C2 domain in a manner similar to that originally identified for Ca2+ dependent PKCs. As demonstrated herein, Alix can rescue defective budding from L domain mutants of VP40 that prevent it from interacting with Nedd4 and Tsg101.

The hypothesis that Ca²⁺ regulation of Nedd4-ependent ubiquitination of VP40 facilitates the L-domain dependent interactions with Nedd4 and Tsg101 required for subsequent interactions with Alix is thus tested. For that, endogenous WT Nedd4 and rescue cells is first suppressed with a WT Nedd4 or a C2 domain mutant that cannot bind Ca²⁺. Immunoprecipitation analysis is then performed to examine the role of Ca²⁺ binding to Nedd4 on Nedd4 and Tsg101 interactions with VP40. In the event that VP40 binds to either Nedd4 or Tsg101 in cells rescued with Nedd4 C2 mutants, then the general role of Ca²⁺ in Tsg101:VP40 L domain interactions is tested by culturing cells in Ca²⁺-free medium during the course of VP40 expression and VLP production, or treating them with Orai1 inhibitors 2-APB, Synta66, or R02959. Tsg101 is be quantified in VP40 precipitates by Western analysis to assess the control of this interaction. Tsg101 and VP40 may have a low affinity or interact transiently, as it is difficult to co-precipitate them from cells. Therefore, an alternative approach has been developed with FLIM to obtain dynamic measurements of the Ca²⁺ and Nedd4 dependence of VP40-GFP and Tsg101-mCherry interactions (FIGS. 19A-19B). The general approach is the same as that used to obtain long-term Ca²⁺ measurements (FIG. 3). Cells will be imaged for 16-24 hours, beginning 4 hours post transfection, in an environmental chamber mounted on the stage of the FLIM microscope. The role of Nedd4 in VP40:Tsg101 interactions and localization will be examined in Nedd4 suppressed cells, and the Ca²⁺ dependence of Nedd4 effects assessed by rescuing suppressed cells with C2 domain mutants, blocking Orai1, or by performing measurements in Ca²⁺ free medium.

### Hypothesis: VP40-Mediated Ca²⁺ Signals Regulate Alix and ALG-2 Plasma Membrane Localization.

While HIV-1 Gag mediated VLP formation involves Alix binding to its YpxL L-domain motif, a similar interaction and function for Alix in VP40-mediated VLP formation has not previously been identified. Alix does indeed play a role in VP40-mediated VLP production (FIG. 16A); Alix can rescue defective VLP production from a double L-domain mutant of VP40 (VP40-APT/PY mutant with PTAP and PPxY motifs deleted), which cannot interact with Tsg101 or Nedd4 (FIG. 16B).

Interestingly, a truncated Alix Bro1-V fragment (minus PRD) efficiently rescues VLP formation more efficiently than full length Alix (FIG. 16B). This rescue involves Alix binding to VP40 via a novel Ypx(n)L-type L domain in VP40 spanning amino acids 18-26. Indeed, Ca²⁺ plays at least two roles in Alix dependent VLP formation. The first mechanism is indicated by the finding that Alix Bro1-V rescue of VLP production is Orai1-dependent (2-APB sensitive, FIG. 16C). Thus, while Ca²⁺ controls a step distal to VP40:Alix binding, it also promotes the rescue of defective eVP40-APT/PY mediated VLP formation by full length Alix (FIG. 16D). Together, these results suggest that the Alix PRD stabilizes a folded, inactive conformation, that masks the Bro1 and V domains that are critical for binding VP40 (via the YPxL motif) to facilitate budding. Thus, Ca²⁺ may control Alix unfolding and activation during the budding process.

The Ca²⁺ dependence of Alix interactions with Tsg101 and ALG-2 at the plasma membrane is defined by measuring these interactions in the presence or absence of STIM1/Orai1 mediated Ca²⁺ signaling. VP40 mediated Ca²⁺ entry is prevented by STIM1 suppression, genetic inactivation (Orai1 E106A HEK293T cells), or pharmacologic inhibition of Orai1. Initially Alix-ESCRT interactions are assessed by coimmunoprecipitation with commercial antibodies for Alix, ALG-2, and Tsg101. This biochemical approach is routinely used to detect strong protein interactions, whereas more subtle, dynamic/transient associations, such as those observed between VP40 and Tsg101, may prompt the implementation of a BiMolecular Complementation (BiMC) assay with split YFP proteins of interest to visualize these interactions. However, as BiMC is irreversible it cannot be used to assess the dynamic equilibrium of transient low affinity protein interactions. Consequently, FLIM is used to perform real time kinetic measurements of Alix:Tsg101:ALG-2 interactions, localization of these interactions, and their dynamic regulation (association and dissociation) by Ca²⁺ (FIGS. 19A-19B) between mCherry-Tsg101 or mCherry-ALG-2 interactions and GFP-Alix (Addgene) in HEK293T cells. While FLIM is optimal for measuring dynamic protein-protein interactions and also provides information about localization, TIRF microscopy can better resolve plasma membrane associated events. Thus, TIRF is utilized to visualize the kinetics and control by Ca²⁺ of Alix and ALG-2 localization to the plasma membrane.

Without wishing to be limited by any theory, Ca²⁺ may regulate the conformation of full length Alix; in the absence of a sufficient Ca²⁺ signal, truncated Alix Bro I-V rescues budding more efficiently. To address this, it is investigated whether mobilizing Ca²⁺ with ionomycin allows rescue of VP40-APT/PY-mediated VLP formation by full length Alix. Interestingly, addition of ionomycin resulted in a 3-fold enhancement of VP40-APT/PY VLP budding by full-length Alix compared to controls; however, ionomycin did not enhance rescue of VP40-APT/PY VLP budding mediated by the Bro1-V fragment of Alix (FIG. 15). Thus, these data support the hypothesis that Ca²⁺ triggers a conformational change critical to Alix function. Moreover, Ca²⁺ may also play additional and distinct conformation independent roles in subsequent Alix-dependent steps of VLP production that could include binding to VP40, membrane localization, or subsequent interactions.

### Example 17: Mechanism of TRPML1 Activation and its Role in Filovirus Egress

### Role of TRPML1 Lipase Activity and Cation Permeabilty in VP40 Mediated VLP Formation.

In addition to its Ca²⁺ permeability, TRPML1 also exhibits a lipase activity that drives generation of TVS14. However, cation permeation of TRPML1 could also produce localized cytoplasmic Ca²⁺ bursts and these may regulate the assembly of the TRPML1:ALG-2:Alix complex, the formation of virus buds, or scission of these VLPs or virions. The role of TRPML1 lipase and channel activity during VLP formation are thus investigated. To assess the role of the ALG-2 complex members in TRPML1 activation and VLP production, ALG-2, Alix, Tsg101, and Nedd4 expression are suppressed, and the consequences of each in TRPML1 localization and activation are examined by TIRF microscopy.

GFP-tagged wild type (WT-MLN1-GFP), lipase deficient (SL-MLN1-GFP), and channel pore defective (F465L-MLN1-GFP) TRPML1 variants are obtained. To determine whether TRPML1 lipase activity or ion permeation regulates VLP formation, endogenous TRPML1 is first suppressed with siRNA (FIG. 15) and after 24 hours cells are transfected with VP40 and WT, lipase deficient, or pore mutant TRPML1. Standard confocal microscopy is used initially to visualize TRMPL1 localization and the localization and size of TVS/VLPs is assessed by TIRF microscopy. In support, TIRF experiments demonstrate that VP40 induced plasma membrane projections can be visualized in real time (FIG. 20). This approach also allows for measuring VLP dynamics and, together with VLP budding assays, revealing the respective roles for TRPML1 lipase and channel activity in virus budding. The initial Ca²⁺ measurements suggest that STIM1/Orai1 are required for VP40 mediated signaling, however, given that TRPML1 is itself Ca²⁺-permeant, it could participate in localized or global changes in cytoplasmic Ca²⁺.

Current approaches should enable to distinguish between alternative Ca²⁺-dependent steps in VP40 VLP egress. For example, while Ca²⁺ may be critical for recruitment of ESCRT proteins to the plasma membrane, it is also possible that VP40 mediated ESCRT complex recruitment occurs in a Ca²⁺-independent manner and that Ca²⁺ is only required for the formation of VLPs. These differences are evident from the measurements. FIG. 6 illustrates Synta66-dependent inhibition of EBOV VP40 protrusions from the plasma membrane, even when total cellular VP40 levels are comparable. This is consistent with the model that VLPs begin to form at the plasma membrane but are unable to complete the budding or scission process. The inability to form VLPs could reflect defective membrane recruitment of Alix, Tsg101, or ALG-2 and defective activation of the scission process. If no role for Ca²⁺ is observed in the formation of VLPs at the membrane, Alix-ESCRT protein interactions, or plasma membrane recruitment, then the focus shifts to Ca²⁺ control of scission mechanisms.

VP40-induced membrane projections from the cell were visualized with TIRF (FIG. 18), but the cross sectional dimension of EBOV VLPs (˜80 nm) is close to the Z-axis resolution of our TIRF system (˜70 nM). If Ca²⁺-induced differences in VLP structures are not quantifiable with TIRF, electron microscopy, which provides Angstrom level resolution, may be sued. In certain embodiments, the experiments outlined are carried out by transient VP40 transfection. However, this may limit the temporal resolution of VP40 activated steps. Mammalian cell lines stably expressing inducible EBOV proteins may provide a way to better analyze the kinetic features of VLP budding. Therefore, a GFP-VP40 construct containing a destablization domain based on the 12-kDa FKBP (FK506 binding protein) appended to the VP40 N-terminus is prepared. This destabilization domain (DD) can be blocked with Shield 1 to control the timing and levels of VP40 expression. Also prepared are stable cell lines that express this inducible construct to control expression of VP40 (pTuner System, Clonetech) and accurately define the kinetics or VP40-dependent steps that control VLP formation.

TABLE 1
Selected Compounds Useful Within the Methods of Invention
Nomenclature

N-(3-methoxyphenyl)-5-(1-methyl-1H-pyrazol-4-yl)-2-furamide
N-{4-[(2,2-dimethylpropanoyl)amino]-3-methoxyphenyl}-4H-
thieno[3,2-b]pyrrole-5-carboxamide
N-(2-fluorophenyl)-5-(1H-pyrazol-4-yl)-2-furamide
N-1,3-benzodioxo1-5-yl-5-(1H-pyrazol-4-yl)-2-furamide
1-methyl-N-(5-phenyl-1,3,4-thiadiazol-2-yl)-9H-beta-carboline-3-
carboxamide
2-{[4-(difluoromethoxy)benzoyl]amino}-5-phenyl-3-
thiophenecarboxamide
2-(2-naphthoylamino)-5-phenyl-3-thiophenecarboxamide
2,5-dimethyl-N-(5-phenyl-1,3,4-thiadiazol-2-yl)-3-furamide
3-(2-chloro-6-fluorophenyl)-5-methyl-N-(5-phenyl-1,3,4-thiadiazol-2-
yl)-4-isoxazolecarboxamide
N-(5-phenyl-1,3,4-thiadiazol-2-yl)-4,5,6,7-tetrahydro-1-benzothiophene-
3-carboxamide
N-[5-(2-chloro-4-fluorophenyl)-1,3,4-thiadiazol-2-yl]-2-
methylbenzamide
N-[5-(3,4-dimethylphenyl)-1,3,4-thiadiazol-2-yl]-2-methylbenzamide
3-bromo-N-[5-(2-chlorophenyl)-1,3,4-thiadiazol-2-yl]benzamide
N-[5-(3-butoxyphenyl)-1,3,4-thiadiazol-2-yl]-2-thiophenecarboxamide
N-[5-(2-chlorophenyl)-1,3,4-thiadiazol-2-yl]-2-methoxybenzamide
N-[5-(2-chloro-4-fluorophenyl)-1,3,4-thiadiazol-2-yl]-2-furamide
2-methoxy-N-[5-(2-methoxyphenyl)-1,3,4-thiadiazol-2-yl]benzamide
4-chloro-N-[5-(3-methylphenyl)-1,3,4-thiadiazol-2-yl]benzamide
N-[5-(3-methoxyphenyl)-1,3,4-thiadiazol-2-yl]-2-thiophenecarboxamide
N-[5-(2-methoxyphenyl)-1,3,4-thiadiazol-2-yl]-2-furamide
N-[5-(4-methoxyphenyl)-1,3,4-thiadiazol-2-yl]-4,5,6,7-tetrahydro-1-
benzothiophene-3-carboxamide
4-ethyl-N-[5-(4-methoxyphenyl)-1,3,4-oxadiazol-2-yl]-5-methyl-3-
thiophenecarboxamide
N-[5-(4-methoxyphenyl)-1,3,4-thiadiazol-2-yl]-2-methyl-3-furamide
methyl 2-[(2-methyl-3-furoyl)amino]-5-phenyl-3-thiophenecarboxylate
N-[5-(2-chlorophenyl)-1,3,4-oxadiazol-2-yl]-2-thiophenecarboxamide
N-[5-(4-pyridinyl)-1,3,4-thiadiazol-2-yl]-4,5,6,7-tetrahydro-1-
benzothiophene-2-carboxamide
2,5-dichloro-N-[5-(4-methoxyphenyl)-1,3,4-thiadiazol-2-yl]benzamide
N-[5-(4-methoxyphenyl)-1,3,4-thiadiazol-2-yl]-4,5,6,7-tetrahydro-1-
benzothiophene-2-carboxamide
5-methyl-N-[5-(4-pyridinyl)-1,3,4-thiadiazol-2-yl]-2-furamide
3-chloro-N-[5-(4-methoxyphenyl)-1,3,4-thiadiazol-2-yl]-1-
benzothiophene-2-carboxamide
N-[5-(4-methoxyphenyl)-1,3,4-thiadiazol-2-yl]-5-methyl-3-
isoxazolecarboxamide
N-[5-(4-pyridinyl)-1,3,4-thiadiazol-2-yl]-2-thiophenecarboxamide
N-[5-(2-chlorophenyl)-1,3,4-oxadiazol-2-yl]-5-methyl-3-
thiophenecarboxamide
methyl 2-(2-furoylamino)-5-phenyl-3-thiophenecarboxylate
N-[5-(4-methoxyphenyl)-1,3,4-thiadiazol-2-yl]-3,5-dimethyl-4-
isoxazolecarboxamide
5-methyl-N-[5-(4-pyridinyl)-1,3,4-thiadiazol-2-yl]-3-
thiophenecarboxamide
N-[5-(4-methoxyphenyl)-1,3,4-thiadiazol-2-yl]-5-methyl-2-furamide
4-methyl-5-phenyl-N-[5-(4-pyridinyl)-1,3,4-thiadiazol-2-yl]-2-
thiophenecarboxamide
N-[5-(2-chlorophenyl)-1,3,4-oxadiazol-2-yl]nicotinamide
N-[5-(4-methoxyphenyl)-1,3,4-thiadiazol-2-yl]-5-methyl-3-
thiophenecarboxamide
N-[5-(3-pyridinyl)-1,3,4-thiadiazol-2-yl]-2-thiophenecarboxamide
methyl 5-phenyl-2-[(2-pyrazinylcarbonyl)amino]-3-thiophenecarboxylate
5-methyl-3-phenyl-N-[5-(4-pyridinyl)-1,3,4-thiadiazol-2-yl]-4-
isoxazolecarboxamide
3-chloro-N-[5-(4-pyridinyl)-1,3,4-thiadiazol-2-yl]-1-benzothiophene-2-
carboxamide
5-bromo-N-[5-(4-methoxyphenyl)-1,3,4-thiadiazol-2-yl]-2-
thiophenecarboxamide
N-[5-(4-pyridinyl)-1,3,4-thiadiazol-2-yl]-4,5,6,7-tetrahydro-1-
benzothiophene-3-carboxamide
N-[5-(2-chlorophenyl)-1,3,4-oxadiazol-2-yl]-2-furamide
1-ethyl-N-[5-(4-methoxyphenyl)-1,3,4-thiadiazol-2-yl]-1H-pyrazole-3-
carboxamide
N-[5-(4-methoxyphenyl)-1,3,4-thiadiazol-2-yl]-1-methyl-1H-pyrazole-5-
carboxamide
2-chloro-4-fluoro-N-[5-(4-pyridinyl)-1,3,4-thiadiazol-2-yl]benzamide
2,4,6-trimethyl-N-[5-(4-pyridinyl)-1,3,4-thiadiazol-2-yl]benzamide
methyl 2-{[(1-methyl-1H-pyrazol-5-yl)carbonyl]amino}-5-phenyl-3-
thiophenecarboxylate
3-phenoxy-N-[5-(4-pyridinyl)-1,3,4-thiadiazol-2-yl]benzamide
4-chloro-N-[5-(4-methoxyphenyl)-1,3,4-thiadiazol-2-yl]-1-methyl-1H-
pyrazole-5-carboxamide
2-ethoxy-N-[5-(4-pyridinyl)-1,3,4-thiadiazol-2-yl]benzamide
3-isopropoxy-N-[5-(4-pyridinyl)-1,3,4-thiadiazol-2-yl]benzamide
2,4-dimethoxy-N-[5-(4-pyridinyl)-1,3,4-thiadiazol-2-yl]benzamide
3-ethoxy-N-[5-(4-pyridinyl)-1,3,4-thiadiazol-2-yl]benzamide
3-methyl-N-[5-(4-pyridinyl)-1,3,4-thiadiazol-2-yl]benzamide
4-bromo-N-[5-(4-pyridinyl)-1,3,4-thiadiazol-2-yl]benzamide
N-[5-(4-pyridinyl)-1,3,4-thiadiazol-2-yl]-1-naphthamide
4-methoxy-N-[5-(4-pyridinyl)-1,3,4-thiadiazol-2-yl]benzamide
4-ethyl-N-[5-(4-pyridinyl)-1,3,4-thiadiazol-2-yl]benzamide
2-methyl-N-[5-(4-pyridinyl)-1,3,4-thiadiazol-2-yl]benzamide
2-bromo-N-[5-(4-pyridinyl)-1,3,4-thiadiazol-2-yl]benzamide
N-[5-(4-pyridinyl)-1,3,4-thiadiazol-2-yl]-3-(trifluoromethyl)benzamide
2,6-difluoro-N-[5-(4-pyridinyl)-1,3,4-thiadiazol-2-yl]benzamide
2-methoxy-N-[5-(4-pyridinyl)-1,3,4-thiadiazol-2-yl]benzamide
3-butoxy-N-[5-(4-pyridinyl)-1,3,4-thiadiazol-2-yl]benzamide
N-[5-(4-pyridinyl)-1,3,4-thiadiazol-2-yl]-2-naphthamide
5-chloro-2-methoxy-N-[5-(4-pyridinyl)-1,3,4-thiadiazol-2-yl]benzamide
5-bromo-2-chloro-N-[5-(4-pyridinyl)-1,3,4-thiadiazol-2-yl]benzamide
4-ethyl-5-methyl-N-[5-(3-pyridinyl)-1,3,4-thiadiazol-2-yl]-3-
thiophenecarboxamide
2-chloro-4,5-difluoro-N-[5-(4-pyridinyl)-1,3,4-thiadiazol-2-yl]benzamide
4-propyl-N-[5-(4-pyridinyl)-1,3,4-thiadiazol-2-yl]benzamide
3,4-dimethyl-N-[5-(4-pyridinyl)-1,3,4-thiadiazol-2-yl]benzamide
N-[5-(2-methoxyphenyl)-1,3,4-thiadiazol-2-yl]-2-pyridinecarboxamide
N-(4-ethylphenyl)-5-(2-thienyl)-3-isoxazolecarboxamide
5-(2-furyl)-N-(4-methoxyphenyl)-3-isoxazolecarboxamide
N-(4-cyanophenyl)-5-(2-thienyl)-3-isoxazolecarboxamide
N-(2,6-dichlorophenyl)-5-(2-thienyl)-3-isoxazolecarboxamide
N-(4-acetylphenyl)-5-(2-thienyl)-3-isoxazolecarboxamide
N-[2-chloro-5-(trifluoromethyl)phenyl]-5-(2-thienyl)-3-
isoxazolecarboxamide
N-(3-chlorophenyl)-5-(2-furyl)-3-isoxazolecarboxamide
5-(2-thienyl)-N-[2-(trifluoromethyl)phenyl]-3-isoxazolecarboxamide
N-[2-chloro-5-(trifluoromethyl)phenyl]-5-(2-furyl)-3-
isoxazolecarboxamide
N-(3-chloro-2-methylphenyl)-5-(2-furyl)-3-isoxazolecarboxamide
N-(3-chloro-4-fluorophenyl)-5-(2-furyl)-3-isoxazolecarboxamide
N-(2-bromo-4,6-difluorophenyl)-5-(2-furyl)-3-isoxazolecarboxamide
N-(5-chloro-2-methoxyphenyl)-5-(2-thienyl)-3-isoxazolecarboxamide
N-(4-acetylphenyl)-5-(2-furyl)-3-isoxazolecarboxamide
N-(4-methoxyphenyl)-5-(2-thienyl)-3-isoxazolecarboxamide
N-(3-chloro-2-methylphenyl)-5-(2-thienyl)-3-isoxazolecarboxamide
N-(3,4-dimethylphenyl)-5-(2-furyl)-3-isoxazolecarboxamide
5-(2-furyl)-N-[2-(methylthio)phenyl]-3-isoxazolecarboxamide
N-(2-ethylphenyl)-5-(2-thienyl)-3-isoxazolecarboxamide
N-[2-(methylthio)phenyl]-5-(2-thienyl)-3-isoxazolecarboxamide
N-(3-chloro-4-fluorophenyl)-5-(2-thienyl)-3-isoxazolecarboxamide
N-(4-cyanophenyl)-5-(2-furyl)-3-isoxazolecarboxamide
N-1,3-benzodioxo1-5-yl-5-(2-furyl)-3-isoxazolecarboxamide
5-(2-furyl)-N-(4-methylphenyl)-3-isoxazolecarboxamide
5-(2-furyl)-N-[2-(trifluoromethyl)phenyl]-3-isoxazolecarboxamide
N-(2-ethylphenyl)-5-(2-furyl)-3-isoxazolecarboxamide
N-(3,4-dimethoxyphenyl)-5-(2-thienyl)-3-isoxazolecarboxamide
N-(3-acetylphenyl)-5-(2-thienyl)-3-isoxazolecarboxamide
N-(2,4-difluorophenyl)-5-(2-furyl)-3-isoxazolecarboxamide
N-(3,4-dimethoxyphenyl)-5-(2-furyl)-3-isoxazolecarboxamide
N-(2-bromo-4,6-difluorophenyl)-5-(2-thienyl)-3-isoxazolecarboxamide
5-(2-furyl)-N-[3-(trifluoromethyl)phenyl]-3-isoxazolecarboxamide
N-(5-chloro-2-methoxyphenyl)-5-(2-furyl)-3-isoxazolecarboxamide
N-(8-ethoxy-5-quinolinyl)-5-(2-thienyl)-3-isoxazolecarboxamide
5-(2-furyl)-N-[4-(trifluoromethoxy)phenyl]-3-isoxazolecarboxamide
5-(2-thienyl)-N-[4-(trifluoromethoxy)phenyl]-3-isoxazolecarboxamide
5-(2-thienyl)-N-[3-(trifluoromethyl)phenyl]-3-isoxazolecarboxamide
N-(4-methyl-3-oxo-3,4-dihydro-2H-1,4-benzoxazin-6-yl)-5-(2-thienyl)-3-
isoxazolecarboxamide
N-(4-ethyl-3-oxo-3,4-dihydro-2H-1,4-benzoxazin-6-yl)-5-(2-thienyl)-3-
isoxazolecarboxamide
methyl 2-({[5-(2-furyl)-3-isoxazolyl]carbonyl}amino)benzoate
N-{4-[2,2-dimethylpropanoyl)amino]-3-methylphenyl}-1′,5′-dimethyl-
1′H,2H,3,4′-bipyrazole-5-carboxamide
N-[4-(2-oxo-2H-chromen-3-yl)phenyl]-1H-1,2,4-triazole-3-carboxamide
2-chloro-N-[4-(1H-tetrazol-1-yl)phenyl]benzamide
4-tert-butyl-N-[4-(1H-tetrazol-1-yl)phenyl]benzamide
N-[4-(4-chloro-1H-pyrazol-1-yl)phenyl]benzamide
N-[4-(3-methyl-1H-pyrazol-1-yl)phenyl]thiophene-2-carboxamide
N-[4-(4-iodo-1H-pyrazol-1-yl)phenyl]nicotinamide
N-[4-(4-chloro-3,5-dimethyl-1H-pyrazol-1-yl)phenyl]-2-furamide
N-[4-(4-chloro-1H-pyrazol-1-yl)phenyl]thiophene-2-carboxamide
N-[4-(4-bromo-3,5-dimethyl-1H-pyrazol-1-yl)phenyl]pyridine-2-
carboxamide
N-[4-(4-chloro-3,5-dimethyl-1H-pyrazol-1-yl)phenyl]isonicotinamide
N-[4-(4-bromo-1H-pyrazol-1-yl)phenyl]nicotinamide
N-[4-(4-bromo-3,5-dimethyl-1H-pyrazol-1-yl)phenyl]nicotinamide
N-[4-(4-chloro-3,5-dimethyl-1H-pyrazol-1-yl)phenyl]pyrazine-2-
carboxamide
N-[4-(4-bromo-1H-pyrazol-1-yl)phenyl]isonicotinamide
N-[4-(4-chloro-3,5-dimethyl-1H-pyrazol-1-yl)phenyl]pyridine-2-
carboxamide
N-[4-(4-bromo-1H-pyrazol-1-yl)phenyl]pyridine-2-carboxamide
N-[4-(4-chloro-1H-pyrazol-1-yl)phenyl]nicotinamide
N-[4-(4-chloro-3,5-dimethyl-1H-pyrazol-1-yl)phenyl]nicotinamide
N-[4-(4-bromo-3,5-dimethyl-1H-pyrazol-1-yl)phenyl]pyrazine-2-
carboxamide
N-[4-(4-chloro-1H-pyrazol-1-yl)phenyl]isonicotinamide
N-[4-(4-chloro-1H-pyrazol-1-yl)phenyl]-2-fluorobenzamide
1-(4-ethylphenyl)-5-methyl-N-[4-(5-methyl-1H-1,2,3-triazol-1-
yl)phenyl]-1H-1,2,3-triazole-4-carboxamide
1-(4-methoxyphenyl)-5-methyl-N-[4-(5-methyl-1H-1,2,3-triazol-1-
yl)phenyl]-1H-1,2,3-triazole-4-carboxamide
N-[4-(5-methyl-1H-1,2,3-triazol-1-yl)phenyl]-2-thiophenecarboxamide
N-[4-(1H-tetrazol-1-yl)phenyl]-1-benzofuran-2-carboxamide
5-methyl-1-(4-methylphenyl)-N-[4-(5-methyl-1H-1,2,3-triazol-1-
yl)phenyl]-1H-1,2,3-triazole-4-carboxamide
N-{4-[5-(ethylthio)-1H-tetrazol-1-yl]phenyl}-1-benzofuran-2-
carboxamide
1-(2-methoxyphenyl)-5-methyl-N-[4-(5-methyl-1H-1,2,3-triazol-1-
yl)phenyl]-1H-1,2,3-triazole-4-carboxamide
5-methyl-N-[4-(5-methyl-1H-1,2,3-triazol-1-yl)phenyl]-1-phenyl-1H-
1,2,3-triazole-4-carboxamide
1-(3-chlorophenyl)-5-methyl-N-[4-(5-methyl-1H-1,2,3-triazol-1-
yl)phenyl]-1H-1,2,3-triazole-4-carboxamide
3-methyl-N-[4-(1H-tetrazol-1-yl)phenyl]-1-benzofuran-2-carboxamide
1-(2-fluorophenyl)-5-methyl-N-[4-(5-methyl-1H-1,2,3-triazol-1-
yl)phenyl]-1H-1,2,3-triazole-4-carboxamide
N-[4-(5-methyl-1H-1,2,3-triazol-1-yl)phenyl]benzamide
1-(2-ethoxyphenyl)-5-methyl-N-[4-(5-methyl-1H-1,2,3-triazol-1-
yl)phenyl]-1H-1,2,3-triazole-4-carboxamide
1-(2-chlorophenyl)-5-methyl-N-[4-(5-methyl-1H-1,2,3-triazol-1-
yl)phenyl]-1H-1,2,3-triazole-4-carboxamide
1-(4-ethoxyphenyl)-5-methyl-N-[4-(5-methyl-1H-1,2,3-triazol-1-
yl)phenyl]-1H-1,2,3-triazole-4-carboxamide
N-[4-(5-methyl-1H-1,2,3-triazol-1-yl)phenyl]-1-naphthamide
4-(5-methyl-1H-1,2,3-triazol-1-yl)-N-[4-(5-methyl-1H-1,2,3-triazol-1-
yl)phenyl]benzamide
1-(4-fluorophenyl)-5-methyl-N-[4-(5-methyl-1H-1,2,3-triazol-1-
yl)phenyl]-1H-1,2,3-triazole-4-carboxamide
1-(2,5-dimethylphenyl)-5-methyl-N-[4-(5-methyl-1H-1,2,3-triazol-1-
yl)phenyl]-1H-1,2,3-triazole-4-carboxamide
N-[4-(1H-tetrazol-1-yl)phenyl]-2-thiophenecarboxamide
1-(2,4-dimethylphenyl)-5-methyl-N-[4-(5-methyl-1H-1,2,3-triazol-1-
yl)phenyl]-1H-1,2,3-triazole-4-carboxamide
N-[4-(5-phenyl-1H-1,2,3-triazol-1-yl)phenyl]benzamide
1-(4-chlorophenyl)-5-methyl-N-[4-(5-methyl-1H-1,2,3-triazol-1-
yl)phenyl]-1H-1,2,3-triazole-4-carboxamide
2-methyl-N-[4-(5-methyl-1H-1,2,3-triazol-1-yl)phenyl]benzamide
1-(3,4-dimethylphenyl)-5-methyl-N-[4-(5-methyl-1H-1,2,3-triazol-1-
yl)phenyl]-1H-1,2,3-triazole-4-carboxamide
N-[4-(5-phenyl-1H-1,2,3-triazol-1-yl)phenyl]-2-thiophenecarboxamide
N-[4-(2-isopropyl-1H-imidazol-1-yl)phenyl]-2,6-dimethoxybenzamide
2-methoxy-N-[2-methoxy-4-(1H-tetrazol-1-yl)phenyl]benzamide
N-[2-methoxy-4-(1H-tetrazol-1-yl)phenyl]-2-thiophenecarboxamide
2,6-difluoro-N-[2-methoxy-4-(1H-tetrazol-1-yl)phenyl]benzamide
N-[4-(2-isopropyl-1H-imidazol-1-yl)phenyl]-2-methoxy-4-
(methylthio)benzamide
5-methyl-N-[4-(1H-tetrazol-1-yl)phenyl]-1-benzofuran-2-carboxamide
N-[4-(3-methyl-1H-pyrazol-1-yl)phenyl]-2-pyridinecarboxamide
N-[2-methoxy-4-(1H-tetrazol-1-yl)phenyl]-2-furamide
3-fluoro-N-[3-methoxy-4-(1H-tetrazol-1-yl)phenyl]benzamide
3,5-dimethoxy-N-[4-(1H-tetrazol-1-yl)phenyl]benzamide
3-chloro-N-[4-(1H-tetrazol-1-yl)phenyl]benzamide
4-chloro-N-[3-methoxy-4-(1H-tetrazol-1-yl)phenyl]benzamide
4-methoxy-N-[4-(1H-tetrazol-1-yl)phenyl]benzamide
N-{4-[2-(4-hydroxyphenyl)-1H-imidazol-1-yl]phenyl}nicotinamide
5-methyl-N-[4-(2-pyridin-4-yl-1H-imidazol-1-yl)phenyl]-3-furamide
N-[2-methyl-4-(6-methyl-9H-purin-9-yl)phenyl]nicotinamide
N-[4-(1H-pyrazol-1-yl)phenyl]-1H-indazole-3-carboxamide
1-{3-methyl-4-[(pyridin-3-ylcarbonyl)amino]phenyl}-1,4,5,6-
tetrahydrocyclopenta[c]pyrazole-3-carboxylic acid
N-[4-(1H,1′H-2,2′-biimidazol-1-yl)phenyl]-4-methylbenzamide
N-[4-(2-cyclopropyl-1H-imidazol-1-yl)phenyl]-5-methyl-3-furamide
N-{4-[2-(3-fluoropyridin-4-yl)-1H-imidazol-1-yl]phenyl}-5-methyl-2-
furamide
N-[2-methyl-4-(2-pyridin-3-yl-1H-imidazol-1-yl)phenyl]-2-furamide
N-[4-(2-methyl-1H-imidazol-1-yl)phenyl]imidazo[1,2-a]pyridine-2-
carboxamide
N-[4-(1′-methyl-1H,1′H-2,2′-biimidazol-1-yl)phenyl]nicotinamide
8-methyl-N-[4-(2-methyl-1H-imidazol-1-yl)phenyl]imidazo[1,2-a]
pyridine-2-carboxamide
N-[4-(2-methyl-1H-imidazol-1-yl)phenyl]-1H-indazole-3-carboxamide
N-{4-[2-(hydroxymethyl)-1H-benzimidazol-1-yl]-2-methylphenyl}
nicotinamide
N-[4-(3,5-dimethyl-1H-pyrazol-1-yl)phenyl]-1H-indazole-3-carboxamide
5-methyl-N-{4-[2-(tetrahydrofuran-3-yl)-1H-imidazaol-1-yl]phenyl}-2-
furamide
N-{4-[2-(2,2-dimethyltetrahydro-2H-pyran-4-yl)-1H-imidazol-1-yl]-2-
methylphenyl}-2-furamide
2-fluoro-N-[4-(2-oxo-2H-chromen-3-yl)phenyl]benzamide
4-ethoxy-N-[4-(2-oxo-2H-chromen-3-yl)phenyl]benzamide
4-fluoro-N-[3-methyl-4-(2-oxo-2H-chromen-3-yl)phenyl]benzamide
2-fluoro-N-[3-methyl-4-(2-oxo-2H-chromen-3-yl)phenyl]benzamide
N-[3-methyl-4-(2-oxo-2H-chromen-3-yl)phenyl]-2-furamide
N,N′-(2,5-pyrimidinediyldi-4,1-phenylene)di(2-furamide)
5-bromo-2-chloro-N-[3-methoxy-4-(2-oxo-2H-chromen-3-yl)
phenyl]benzamide
3-methyl-N-[4-(2-oxo-2H-chromen-3-yl)phenyl]benzamide
3-fluoro-N-[4-(2-oxo-2H-chromen-3-yl)phenyl]benzamide
4-chloro-N-[4-(2-quinoxalinyl)phenyl]benzamide
N-[3-methyl-4-(2-oxo-2H-chromen-3-yl)phenyl]-1,3-benzodioxole-5-
carboxamide
N-[4-(2-oxo-2H-chromen-3-yl)phenyl]-1,3-benzodioxole-5-carboxamide
2-chloro-N-[4-(2-oxo-2H-chromen-3-yl)phenyl]-5-(4H-1,2,4-triazol-4-
yl)benzamide
N-[4-(2-oxo-2H-chromen-3-yl)phenyl]nicotinamide
N-[3-chloro-4-(2-oxo-2H-chromen-3-yl)phenyl]-2-thiophenecarboxamide
N-[3-chloro-4-(2-oxo-2H-chromen-3-yl)phenyl]-2-furamide
N-[3-chloro-4-(2-oxo-2H-chromen-3-yl)phenyl]nicotinamide
3-chloro-N-[3-methoxy-4-(2-oxo-2H-chromen-3-yl)phenyl]-4-
methylbenzamide
4-cyano-2-fluoro-N-[3-methoxy-4-(2-oxo-2H-chromen-3-yl)
phenyl]benzamide
N-(2′,3′,4′-trimethoxy-3-methylbiphenyl-4-yl)nicotinamide
N-[2′-(hydroxymethyl)-3-methylbiphenyl-4-yl]nicotinamide
3-fluoro-N-(4-pyrimidin-5-ylphenyl)benzamide
N-[4-(2-methoxypyridin-3-yl)-2-methylphenyl]nicotinamide
N-[3-methyl-2′-(morpholin-4-ylmethyl)biphenyl-4-yl]nicotinamide
N-[2-methyl-4-(4-pyridinyl)phenyl]nicotinamide
N-[3-methyl-3′-(1H-pyrazol-3-yl)-4-biphenylyl]nicotinamide
4-chloro-N-(5-phenyl-1,3-thiazol-2-yl)benzamide
N-(5-phenyl-1,3-thiazol-2-yl)isonicotinamide
N-(3-acetyl-5-phenyl-1H-pyrrol-2-yl)benzamide
N-[5-(4-methoxyphenyl)-1,3,4-thiadiazol-2-yl]-4-methylbenzamide
4-bromo-N-[5-(4-methoxyphenyl)-1,3,4-thiadiazol-2-yl]benzamide
4-chloro-N-[5-(4-methoxyphenyl)-1,3,4-thiadiazol-2-yl]benzamide
N-[5-(4-methoxyphenyl)-1,3,4-thiadiazol-2-yl]-3-methylbenzamide
N-[5-(1,3-benzodioxo1-5-yl)-1,3,4-thiadiazol-2-yl]-2-furamide
N-[5-(4-methoxyphenyl)-1,3,4-thiadiazol-2-yl]-2-furamide
4-chloro-N-[5-(3-pyridinyl)-1,3,4-thiadiazol-2-yl]benzamide
N-[5-(4-methoxyphenyl)-1,3,4-thiadiazol-2-yl]-2-thiophenecarboxamide
N-[5-(2,4-dichlorophenyl)-1,3,4-thiadiazol-2-yl]-2-thiophenecarboxamide
2,4-dichloro-N-[5-(4-methoxyphenyl)-1,3,4-thiadiazol-2-yl]benzamide
2,4-dichloro-N-[5-(4-methylphenyl)-1,3,4-thiadiazol-2-yl]benzamide
2-methyl-N-[5-(4-methylphenyl)-1,3,4-thiadiazol-2-yl]benzamide
N-[5-(4-methylphenyl)-1,3,4-thiadiazol-2-yl]-2-thiophenecarboxamide
N-[5-(1,3-benzodioxo1-5-yl)-1,3,4-thiadiazol-2-yl]-3-chloro-1-
benzothiophene-2-carboxamide
2-methoxy-N-[5-(4-methoxyphenyl)-1,3,4-thiadiazol-2-yl]benzamide
N-[5-(2,4-dichlorophenyl)-1,3,4-thiadiazol-2-yl]-2-methoxybenzamide
2-bromo-N-[5-(3-pyridinyl)-1,3,4-thiadiazol-2-yl]benzamide
N-[5-(4-tert-butylphenyl)-1,3,4-thiadiazol-2-yl]-2-furamide
N-[5-(4-tert-butylphenyl)-1,3,4-thiadiazol-2-yl]-2-thiophenecarboxamide
2,4-dichloro-N-[5-(4-fluorophenyl)-1,3,4-thiadiazol-2-yl]benzamide
N-[5-(4-fluorophenyl)-1,3,4-thiadiazol-2-yl]-2-thiophenecarboxamide
N-[5-(3,4-dichlorophenyl)-1,3,4-thiadiazol-2-yl]-3-methoxybenzamide
N-[5-(3,4-dichlorophenyl)-1,3,4-thiadiazol-2-yl]-2-furamide
4-chloro-N-[5-(4-fluorophenyl)-1,3,4-thiadiazol-2-yl]benzamide
4-fluoro-N-[5-(2-methoxyphenyl)-1,3,4-thiadiazol-2-yl]benzamide
2-fluoro-N-[5-(4-methoxyphenyl)-1,3,4-thiadiazol-2-yl]benzamide
3-ethoxy-N-[5-(3-ethoxyphenyl)-1,3,4-thiadiazol-2-yl]benzamide
N-[5-(4-ethoxyphenyl)-1,3,4-thiadiazol-2-yl]-2-fluorobenzamide
N-[5-(3-ethoxyphenyl)-1,3,4-thiadiazol-2-yl]-2-fluorobenzamide
3-ethoxy-N-[5-(2-methylphenyl)-1,3,4-thiadiazol-2-yl]benzamide
N-[5-(3-ethoxyphenyl)-1,3,4-thiadiazol-2-yl]-2-furamide
ethyl 2-(isonicotinoylamino)-5-phenyl-3-thiophenecarboxylate
2-chloro-N-[5-(4-chlorophenyl)-1,3,4-thiadiazol-2-yl]benzamide
N-[5-(4-chlorophenyl)-1,3,4-thiadiazol-2-yl]-2-thiophenecarboxamide
3-amino-4,6-dimethyl-N-(5-phenyl-1,3,4-thiadiazol-2-yl)thieno[2,3-b]
pyridine-2-carboxamide
3-amino-N-(5-phenyl-1,3,4-thiadiazol-2-yl)-6,7-dihydro-5H-
cyclopenta[b]thieno[3,2-e]pyridine-2-carboxamide
N-[3-(aminocarbonyl)-5-phenyl-2-thienyl]-2-furamide
3-amino-N-(5-phenyl-1,3,4-thiadiazol-2-yl)-5,6,7,8-tetrahydrothieno
[2,3-b]quinoline-2-carboxamide
N-[3-cyano-5-(4-methoxyphenyl)-2-furyl]-2-thiophenecarboxamide
N-(3-cyano-5-phenyl-2-furyl)-2-furamide
N-[5-(4-bromophenyl)-1,3,4-thiadiazol-2-yl]-2-fluorobenzamide
N-[3-(aminocarbonyl)-5-phenyl-2-thienyl]-2-thiophenecarboxamide
2-[(2-fluorobenzoyl)amino]-5-phenyl-3-thiophenecarboxylic acid

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing a viral infection in a subject in need thereof, the method comprising administering to subject an effective amount of at least one inhibitor of a channel selected from the group consisting of calcium-release activated calcium (CRAC) channel and transient receptor potential mucolipin I (TRPML1) channel, whereby the viral infection is treated or prevented in the subject.

2. The method of claim 1, wherein administration of the inhibitor blocks, inhibits or interferes with viral spread or viral trafficking within the subject or to another subject.

3. The method of claim 1, wherein administration of the inhibitor blocks, inhibits or interferes with viral budding within the subject.

4. The method of claim 1, wherein administration of the inhibitor blocks, inhibits or interferes with virus dissemination within the subject or to another subject.

5. The method of claim 1, wherein administration of the inhibitor blocks, inhibits or interferes with viral disease progression in the subject or viral disease transmission within the subject or to another subject.

6. The method of claim 1, wherein the virus is selected from the group consisting of a filovirus, arenavirus, rhabdovirus, paramyxovirus, retrovirus, orthomyxovirus, and any combinations thereof.

7. The method of claim 6, wherein the virus is selected from the group consisting of Influenza A, Influenza B, Influenza C, Junin, Ebola, Marburg, Lassa fever, rabies, vesicular stomatitis, emerging lyssavirus, Nipah, Hendra, HIV-1, HIV-2, HTLV-1, and any combinations thereof.

8. The method of claim 1, wherein the inhibitor, or a salt or solvate thereof, is at least one selected from the group consisting of:
lanthanides;
N-(4-(3,5-bis(trifluoromethyl)-1H-pyrazol-1-yl)phenyl)-4-methyl-1,2,3-thiadiazole-5-carboxamide (Pyr2/BTP2/YM58483);
ethyl 1-(4-(2,3,3-trichloroacrylamido)phenyl)-5-(trifluoromethyl)-1H-pyrazole-4-carboxylate (Pyr3);
N-(4-(3,5-bis(trifluoromethyl)-1H-pyrazol-1-yl)phenyl)-3-fluoroisonicotinamide (Pyr6);
N-(4-(3,5-bis(trifluoromethyl)-1H-pyrazol-1-yl)phenyl)-4-methylbenzenesulfonamide (Pyr10);
2-aminoethoxydiphenylborate (2-APB);
2,2′-((((oxybis(methylene))bis(3,1-phenylene))bis(phenylboranediyl))bis(oxy)) bis(ethan-1-amine) (DPB162-AE);
2,2′-((((oxybis(methylene))bis(4,1-phenylene))bis(phenylboranediyl))bis(oxy))bis (ethan-1-amine) (DPB163-AE);
capsaicin;
5-nitro-2-(3-phenylpropylamino)-benzoic acid (NPPB);
diethylstilbestrol (DES);
bromenol lactone (BEL);
CM2489;
CM3457;
cholestatic bile acids;
1-(5-chloronaphthalene-1-sulfonyl)homopiperazine (ML-9);
2,6-difluoro-N-{5-[4-methyl-1-(5-methyl-thiazol-2-yl)-1,2,5,6-tetrahydro-pyridin-3-yl]-pyrazin-2-yl}-benzamide (R02959);
N-(2′,5′-dimethoxy-[1,1′-biphenyl]-4-yl)-3-fluoroisonicotinamide (Synta-66 or Synta66);
2,6-difluoro-N-(1-(2-phenoxybenzyl)-1H-pyrazol-3-yl)benzamide (GSK-5503A);
2,6-difluoro-N-(1-(4-hydroxy-2-(trifluoromethyl)benzyl)-1H-pyrazol-3-yl)benzamide (GSK-7975A);
4-[3-(diphenylmethyl)-1,2,4-oxadiazol-5-yl]piperidineyl]piperidine (FCC2121);
3-(4-methyl-1,5-diphenyl-1H-pyrazol-3-yl)-2-phenylpropanoic acid (FCC2122);
N-[1-({2-Chloro-5-[(cyclopropylmethyl)oxy]phenyl}methyl)-1H-pyrazol-3-yl]-2,6-difluorobenzamide;
N-{1-[(2,4-Dichlorophenyl)methyl]-1H-pyrazol-3-yl}-2,6-difluorobenzamide;
2-Bromo-N-{1-[(2,4-dichlorophenyl)methyl]-1H-pyrazol-3-yl}-6-fluorobenzamide;
2-Chloro-N-{1-[(2,4-dichlorophenyl)methyl]-1H-pyrazol-3-yl}-6-fluorobenzamide;
2,6-Dichloro-N-{1-[(2,4-dichlorophenyl)methyl]-1H-pyrazol-3-yl}benzamide;
N-{1-[(2,4-dichlorophenyl)methyl]-1H-pyrazol-3-yl}-3, 5-difluoro-4-pyridinecarboxamide;
N-[1-({5-chloro-2-[(phenylmethyl)oxy]phenyl}methyl)-1H-pyrazol-3-yl]-2,6-25 difluorobenzamide;
N-{1-[(2,6-dichlorophenyl)methyl]-1H-pyrazol-3-yl}-2,6-difluorobenzamide;
N-[1-({5-chloro-2-[(2-methylpropyl)oxy]phenyl}methyl)-1H-pyrazol-3-yl]-2,6-difluorobenzamide;
N-(1-{[2-bromo-5-(methyloxy)phenyl]methyl-1H-pyrazol-3-yl)-2,6-difluorobenzamide;
N-(1-{[5-chloro-2-(methyl oxy)phenyl]methyl-}H-pyrazol-3-yl)-2,6-difluorobenzamide;
2,6-Difluoro-N-(1-{[2-(phenyloxy)phenyl]methyl}-1H-pyrazol-3-yl)benzamide;
N-[1-({5-bromo-2-[(phenylmethyl)oxy]phenyl}methyl)-1H-pyrazol-3-yl]-2,6-difluorobenzamide;
2,6-Difluoro-N-[1-({2-[(trifluoromethyl)oxy]phenyl}methyl)-1H-pyrazol-3-yl]benzamide;
2,6-Difluoro-N-(1-{[4-[(phenylmethyl)oxy]-2-(trifluoromethyl)phenyl]methyl}-1H-pyrazol-3-yl)benzamide;
N-{1-[(2-Bromo-6-chlorophenyl)methyl]-1H-pyrazol-3-yl}-2,6-difluorobenzamide;
2,6-Difluoro-/V-[1-({2-[(phenylmethyl)oxy]phenyl}methyl)-1H-pyrazol-3-yl]benzamide;
N/-[1-({2-chloro-5-[(2-methylpropyl)oxy]phenyl}methyl)-1H-pyrazol-3-yl]-2,6-difluorobenzamide;
N-(1-{[4-[(cyclopropylmethyl)oxy]-2-(trifluoromethyl)phenyl]methyl}-1H-pyrazol-3-yl)-2,6-difluorobenzamide;
2,6-Difluoro-N-(1-{[4-iodo-2-(trifluoromethyl)phenyl]methyl-}H-pyrazol-3-yl)benzamide;
2,6-Difluoro-N-(1-{[4-methyl-2-(trifluoromethyl)phenyl]methyl-}H-pyrazol-3-yl)benzamide;
N-(1-{[4-cyclopropyl-2-(trifluoromethyl)phenyl]methyl-}H-pyrazol-3-yl)-2,6-difluorobenzamide;
2,6-Difluoro-N-{1-[(4-iodo-2-methylphenyl)methyl]-1H-pyrazol-3-yl}benzamide;
N-(1-{[4-chloro-2-(trifluoromethyl)phenyl]methyl}-1H-pyrazol-3-yl)-2,6-difluorobenzamide;
2-Fluoro-N-(1-{[4-iodo-2-(trifluoromethyl)phenyl]methyl}-1H-pyrazol-3-yl)benzamide;
2-Chloro-N-(1-{[4-cyclopropyl-2-(trifluoromethyl)phenyl]methyl]-1H-pyrazol-3-yl)benzamide;
N-(1-{[4-cyclopropyl-2-(trifluoromethyl)phenyl]methyl-}H-pyrazol-3-yl)-2-fluorobenzamide;
2,6-Difluoro-N-(1-{[5-iodo-2-(trifluoromethyl)phenyl]methyl-}H-pyrazol-3-yl)benzamide;
2,6-Difluoro-N-(1-{[2-fluoro-6-(trifluoromethyl)phenyl]methyl-}H-pyrazol-3-yl)benzamide;
2,6-Difluoro-N-(1-{[4-hydroxy-2-(trifluoromethyl)phenyl]methyl-}H-pyrazol-3-yl)benzamide;
N-[4-(3,5-dicyclopropyl-1H-pyrazol-1-yl)phenyl]-1H-benzo[d]imidazole-6-carboxamide;
N-[4-(3,5-dicyclopropyl-1H-pyrazol-1-yl)phenyl]-1H-benzo[d][1,2,3]triazole-6-carboxamide;
N-[4-(3,5-dicyclopropyl-1H-pyrazol-1-yl)phenyl]quinoline-6-carboxamide;
N-[4-(3,5-dicyclopropyl-1H-pyrazol-1-yl)phenyl]quinoxaline-6-carboxamide;
2-(1H-benzo[d]imidazol-1-yl)-N-[4-(3,5-dicyclopropyl-1H-pyrazol-1-yl)phenyl] acetamide;
2-(1H-benzo[d][1,2,3] triazol-1-yl)-N-[4-(3,5-dicyclopropyl-1H-pyrazol-1-yl)phenyl] acetamide;
N-[4-(3,5-dicyclopropyl-1H-pyrazol-1-yl)phenyl]-2-(1H-indol-3-yl)acetamide;
N-[4-(3,5-dicyclopropyl-1H-pyrazol-1-yl)phenyl]-2-(imidazo[1,2-a]pyridin-2-yl) acetamide;
N-[4-(3,5-dicyclopropyl-1H-pyrazol-1-yl)phenyl]-2-(quinolin-6-yl)acetamide;
N-[4-(3,5-dicyclopropyl-1H-pyrazol-1-yl)phenyl]-2-(quinolin-6-yl)acetamide;
2-(1H-benzo[d][1,2,3]triazol-1-yl)-N-(4-(3,5-dicyclopropyl-1H-pyrazol-1-yl)-3-fluorophenyl) acetamide;
N-[4-(3,5-dicyclopropyl-1H-pyrazol-1-yl)-3-fluorophenyl]-2-(quinolin-6-yl) acetamide;
N-[6-(3,5-dicyclopropyl-1H-pyrazol-1-yl)pyridin-3-yl]quinoline-6-carboxamide;
N-[6-(3,5-dicyclopropyl-1H-pyrazol-1-yl)pyridin-3-yl]quinoxaline-6-carboxamide;
2-(1H-benzo[d][1,2,3]triazol-1-yl)-N-[6-(3,5-dicyclopropyl-1H-pyrazol-1-yl)pyridin-3-yl]acetamide;
N-[6-(3,5-dicyclopropyl-1H-pyrazol-1-yl)pyridin-3-yl]-2-(quinolin-6-yl)acetamide;
N-{4-[5-cyclopropyl-3-(trifluoromethyl)-1H-pyrazol-1-yl]phenyl}quinoline-6-carboxamide;
N-{4-[5-cyclopropyl-3-(trifluoromethyl)-1H-pyrazol-1-yl]phenyl}quinoxaline-6-carboxamide;
2-(1H-benzo[d]imidazol-1-yl)-N-{4-[5-cyclopropyl-3-(trifluoromethyl)-1H-pyrazol-1-yl]phenyl]acetamide;
2-(1H-benzo[d][1,2,3]triazol-1-yl)-N-{4-[5-cyclopropyl-3-(trifluoromethyl)-1H-pyrazol-1-yl]phenyl} acetamide;
2-(2H-benzo[d][1,2,3]triazol-2-yl)-N-{4-[5-cyclopropyl-3-(trifluoromethyl)-1H-pyrazol-1-yl]phenyl} acetamide;
2-(3H-[1,2,3]triazolo[4, 5-b]pyridin-3-yl)-N-{4-[5-cyclopropyl-3-(trifluoromethyl)-1H-pyrazol-1-yl] phenyl}acetamide;
(S)-2-(3H-[1,2,3]triazolo[4,5-b]pyridin-3-y])-N-{4-[5-cyclopropyl-3-(trifluoromethyl)-1H-pyrazol-1-yl] phenyl} propanamide;
2-(6-amino-9H-purin-9-yl)-N-{4-[5-cyclopropyl-3-(trifluoromethyl)-1H-pyrazol-1-yl]phenyl}acetamide;
N-(4-(5-cyclopropyl-3-(trifluoromethyl)-1H-pyrazol-1-yl)phenyl)-2-(1,3-dimethyl-2,6-di oxo-2, 3-dihydro-1H-purin-7(6H)-yl)acetamide;
N-{4-[5-cyclopropyl-3-(trifluoromethyl)-1H-pyrazol-1-yl)phenyl)-2-(imidazo[1,2-a]pyridin-2-yl) acetamide;
N-{4-[5-cyclopropyl-3-(trifluoromethyl)-1H-pyrazol-1-yl]phenyl}-2-(quinolin-6-yl)acetamide;
N-{4-[5-cyclopropyl-3-(trifluoromethyl)-1H-pyrazol-1-yl]phenyl}-2-(quinolin-6-yl)propanamide;
N-{4-[5-cyclopropyl-3-(trifluoromethyl)-1H-pyrazol-1-yl]-3-fluorophenyl}-1H-benzo[d][1,2,3]triazole-6-carboxamide;
2-(1H-benzo[d][1,2,3]triazol-1-yl)-N-{4-[5-cyclopropyl-3-(trifluoromethyl)-1H-pyrazol-1-yl]-3-fluorophenyl}acetamide;
N-{6-[5-cyclopropyl-3-(trifluoromethyl)-1H-pyrazol-1-yl]pyridin-3-yl}-1H-benzo[d][1,2,3] triazole-5-carboxamide;
2-(1H-benzo[d][1,2,3]triazol-1-yl)-N-{6-[5-cyclopropyl-3-(trifluoromethyl)-1H-pyrazol-1-yl]pyridin-3-yl}acetamide;
2-(2H-benzo[d][1,2,3]triazol-2-yl)-N-{6-[5-cyclopropyl-3-(trifluoromethyl)-1H-pyrazol-1-yl]pyridin-3-yl}acetamide;
N-{6-[5-cyclopropyl-3-(trifluoromethyl)-1H-pyrazol-1-yl]pyridin-3-yl}-2-(quinolin-6-yl)acetamide;
2-(1H-benzo[d][1,2,3]triazol-1-yl)-N-{6-[4-chloro-5-cyclopropyl-3-(trifluoromethyl)-1H-pyrazol-1-yl]pyridin-3-yl}acetamide;
4-[5-cyclopropyl-3-(trifluoromethyl)-1H-pyrazol-1-yl]-3-fluoro-N-(quinolin-6-ylmethyl)benzamide;
1-[4-(3,5-dicyclopropyl-1H-pyrazol-1-yl)phenyl]-3-(quinolin-6-yl)urea;
4-[6-(2-chloro-6-fluoro-phenyl)-5H-pyrrolo[3,2-d]pyrimidin-2-yl]-3,N,N-trimethyl-benzenesulfonamide;
6-(2-Chloro-phenyl)-2-(2-methyl-5-trifluoromethyl-2H-pyrazol-3-yl)-5H-pyrrolo[2,3-b]pyrazine;
4-[6-(2-Chloro-phenyl)-5H-pyrrolo[2,3-b]pyrazin-2-yl]-3-methyl-benzoic acid methyl ester;
4-(6-(2-Chlorophenyl)-5H-pyrrolo[2,3-b]pyrazin-2-yl)-N,N,3-trimethylbenzene sulfonamide;
6-(2-chloro-6-fluorophenyl)-2-(1-methyl-3-(trifluoromethyl)-1H-pyrazol-5-yl)-5H-pyrrolo[2,3-b]pyrazine;
6-Cyclohexyl-2-(1-methyl-3-(trifluoromethyl)-1H-pyrazol-5-yl)-5H-pyrrolo[2,3-b]pyrazine;
4-(6-Cyclohexyl-5H-pyrrolo[2,3-b]pyrazin-2-yl)-N,N,3-trimethylbenzenesulfonamide;
2,6-Difluoro-N-(6-(5-(4-methyl-5-oxo-4,5-dihydro-1,3,4-oxadiazol-2-yl)-3-(trifluoromethyl)-1H-pyrazol-1-yl)pyridin-3-yl)benzamide;
2-Fluoro-6-methyl-N-(6-(5-(4-methyl-5-oxo-4,5-dihydro-1,3,4-oxadiazol-2-yl)-3-(trifluoromethyl)-1H-pyrazol-1-yl)pyridin-3-yl)benzamide;
5-(3-Cyclopropyl-1-(5-((2,6-difluorobenzyl)amino)pyridin-2-yl)-1H-pyrazol-5-yl)-3-methyl-1,3,4-oxadiazol-2(3H)-one;
N-(6-(3-(Difluoromethyl)-5-(4-methyl-5-oxo-4,5-dihydro-1,3,4-oxadiazol-2-yl)-1H-pyrazol-1-yl)pyridin-3-yl)-2,6-difluorobenzamide;
5-(1-(5-((2,6-Difluorobenzyl)amino)pyridin-2-yl)-5-(fluoromethyl)-1H-pyrazol-3-yl)-3-methyl-1,3,4-oxadiazol-2(3H)-one;
Methyl 3-(1-(5-((2,6-difluorobenzyl)amino)pyridin-2-yl)-5-(trifluoromethyl)-1H-pyrazol-3-yl)-5-methyl-4,5-dihydroisoxazole-5-carboxylate;
Methyl 3-(1-(5-((2-chloro-6-fluorobenzyl)amino)pyridin-2-yl)-5-(trifluoromethyl)-1H-pyrazol-3-yl)-5-methyl-4,5-dihydroisoxazole-5-carboxylate;
2,6-Difluoro-W-(6-(3-(4-methyl-5-oxo-4,5-dihydro-1,3,4-oxadiazol-2-yl)-5-(trifluoromethyl)-1H-pyrazol-1-yl)pyridin-3-yl)benzamide;
2-Chloro-6-fluoro-N-(6-(3-(4-methyl-5-oxo-4,5-dihydro-1,3,4-oxadiazol-2-yl)-5-(trifluoromethyl)-\H-pyrazol-1-yl)pyridin-3-yl)benzamide;
2-Fluoro-6-methyl-/v-(6-(3-(4-methyl-5-oxo-4,5-dihydro-1,3,4-oxadiazol-2-yl)-5-(trifluoromethyl)-1H-pyrazol-1-yl)pyridin-3-yl)benzamide;
N-(6-(5-(Difluoromethyl)-3-(4-methyl-5-oxo-4,5-dihydro-1,3,4-oxadiazol-2-yl)-1H-pyrazol-1-yl)pyridin-3-yl)-2,6-difluorobenzamide;
5-(1-(5((2,6-Difluorobenzyl) amino)pyridin-2-yl)-5-(difluoromethyl)-1H-pyrazol-3-yl)-3-methyl-1,3,4-oxadiazol-2(3H)-one;
5-(1-(54(2,6-Difluorobenzyl)amino)pyridin-2-yl)-3-(difluoromethyl)-1H-pyrazol-5-yl)-3-methyl-1,3,4-oxadiazol-2(3H)-one;
N-(6-(3-(5,5-Dimethyl-4-oxo-4,5-dihydroisoxazol-3-yl)-5-(trifluoromethyl)-1H-pyrazol-1-yl)pyridin-3-yl)-2,6-difluorobenzamide;
2-Chloro-N-(6-(3-(5,5-dimethyl-4-oxo-4,5-dihydroisoxazol-3-yl)-5-(trifluoromethyl)-1H-pyrazol-1-yl)pyridin-3-yl)-6-fluorobenzamide;
2,6-Difluoro-N-(6-(1′,4′,4,-trimethyl-5′-oxo-5-(trifluoromethyl)-4′,5′-dihydro-1H,1H′-[3,3′-bipyrazol]-1-yl)pyridin-3-yl)benzamide;
2-Chloro-6-fluoro-N-(6-(1,4,4′-trimethyl-5′-oxo-5-(trifluoromethyl)-4′,5,-dihydro-1H,1′H-[3,3′-bipyrazol]-1-yl)pyridin-3-yl)benzamide;
2-Fluoro-6-methyl-N-(6-(1,4,4′-trimethyl-5′-oxo-5-(trifluoromethyl)-4′,5′-dihydro-1H,1′H-[3,3′-bipyrazol]-1-yl)pyridin-3-yl)benzamide;
2,6-Difluoro-N-(6-(3-(4-methyl-5-oxo-4, 5-dihydro-1,2,4-oxadiazol-3-yl)-5-(trifluoromethyl)-1H-pyrazol-1-yl)pyridin-3-yl)benzamide;
N-(6-(3-(4-Acetyl-5, 5-dimethyl-4,5-dihydro-1,3,4-oxadiazol-2-yl)-5-trifluoromethyl)-1H-pyrazol-1-yl)pyridin-3-yl)-2,6-difluorobenzamide;
N-(6-(3-(4,4-Dimethyl-4, 5-dihydrooxazol-2-yl)-5-(trifluoromethyl)-1H-pyrazol-1-yl)pyridin-3-yl)-2,6-difluorobenzamide;
5-(1-(5-((2,6-Difluorobenzyl)amino)pyridin-2-yl)-5-(trifluoromethyl)-1H-pyrazol-3-yl)-3-methyl-1,3,4-oxadiazol-2(3H)-one;
5-(1-(5-((2-Chloro-6-fluorobenzyl)amino)pyridin-2-yl)-5-(trifluoromethyl)-1H-pyrazol-3-yl)-3-methyl-1,3,4-oxadiazol-2(3iy)-one;
1′-(5-((2,6-Difluorobenzyl)amino)pyridin-2-yl)-1,4,4-trimethyl-5′-(trifluoromethyl)-1H,1′H-[3,3′-bipyrazol]-5(4H)-one;
1′-(5-((2-Chloro-6-fluorobenzyl)amino)pyridin-2-yl)-1,4,4-trimethyl-5′-(trifluoromethyl)-1H,1′H-[3,3′-bipyrazol]-5(4H)-one;
3-(1-(5-((2,6-Difluorobenzyl)amino)pyridin-2-yl)-5-(trifluoromethyl)-1H-pyrazol-3-yl)-4-methyl-1,2,4-oxadiazol-5(4H)-one;
1-(5-(1-(5-((2,6-Difluorobenzyl)amino)pyridin-2-yl)-5-(trifluoromethyl)-1H-pyrazol-3-yl)-2,2-dimethyl-1,3,4-oxadiazol-3 (2H)-yl)ethanone;
N-(2,6-Difluorobenzyl)-6-(3-(4,4-dimethyl-4, 5-dihydrooxazol-2-yl)-5-(trifluoromethyl)-1H-pyrazol-1-yl)pyridin-3-amine;
N-(6-(5-Cyclopropyl-3-(4-methyl-5-oxo-4, 5-dihydro-1,3,4-oxadiazol-2-yl)-1H-pyrazol-1-yl)pyridin-3-yl)-2,6-difluorobenzamide;
N-(6-(3-Cyclopropyl-5-(4-methyl-5-oxo-4, 5-dihydro-1,3,4-oxadiazol-2-yl)-1H-pyrazol-1-yl)pyridin-3-yl)-2,6-difluorobenzamide;
2,6-Difluoro-N-(6-(5-methyl-3-(4-methyl-5-oxo-4, 5-dihydro-1,3,4-oxadiazol-2-yl)-1H-pyrazol-1-yl)pyridin-3-yl)benzamide;
5-(1-(5-((2,6-Difluorobenzyl)amino)pyridin-2-yl)-5-methyl-1H-pyrazol-3-yl)-3-methyl-1,3,4-oxadiazol-2(3H)-one;
(3-(1-(5-((2,6-Difluorobenzyl)amino)pyridin-2-yl)-5-(trifluoromethyl)-1H-pyrazol-3-yl)-5-methyl-4,5-dihydroisoxazol-5-yl)methanol;
(3-(1-(5-((2-Chloro-6-fluorobenzyl)amino)pyridin-2-yl)-5-(trifluoromethyl)-1H-pyrazol-3-yl)-5-methyl-4,5-dihydroisoxazol-5-yl)methanol;
Methyl 3-(1-(5-(2,6-difluorobenzamido)pyridin-2-yl)-5-(trifluoromethyl)-1H-pyrazol-3-yl)-5-methyl-4,5-dihydroisoxazole-5-carboxylate;
2,6-Difluoro-N-(6-(3-(5-(hydroxymethyl)-5-methyl-4,5-dihydroisoxazol-3-yl)-5-(trifluoromethyl)-1H-pyrazol-1-yl)pyridin-3-yl)benzamide;
3-(1-(5-(2,6-Difluorobenzamido)pyridin-2-yl)-5-(trifluoromethyl)-1H-pyrazol-3-yl)-5-methyl-4,5-dihydroisoxazole-5-carboxamide;
2,6-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2-Chloro-6-fluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2-Fluoro-6-methyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2-Fluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2,3-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2,4,5-Trifluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2,3,4-Trifluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2,4-Difluoro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2,3-Dimethyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2-Chloro-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
2-Methyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
4-Ethyl-N-(5-(3-(4-methyl-5-oxo-4,5-dihydro-1,3,4-oxadiazol-2-yl)-5-(trifluoromethyl)-1H-pyrazol-1-yl)pyridin-2-yl)benzamide;
N-(5-(3-(4-Methyl-5-oxo-4,5-dihydro-13,4-oxadiazol-2-yl)-5-(trifluoromethyl)-1H-pyrazol-1-yl)pyridin-2-yl)-2-naphthamide;
5-(1-(6-((2,6-Difluorobenzyl)amino)pyridin-3-yl)-5-(trifluoromethyl)-1H-pyrazol-3-yl)-3-methyl-1,3,4-oxadiazol-2(3H)-one;
5-(1-(6-((2-Chloro-6-fluorobenzyl)amino)pyridin-3-yl)-5-(trifluoromethyl)-1H-pyrazol-3-yl)-3-methyl-1,3,4-oxadiazol-2(3H)-one;
5-(1-(6-((2-Fluoro-6-methylbenzyl)amino)pyridm-3-yl)-5-(trifluoromethyl)-1H-pyrazol-3-yl)-3-methyl-1,3,4-oxadiazol-2(3H)-one;
N-(2,6-Difluorophenyl)-6-(3-(4-methyl-5-oxo-4,5-dihydro-1,3,4-oxadiazol-2-yl)-5-(trifluoromethyl)-1H-pyrazol-1-yl)nicotinamide;
N-(2-Chloro-6-fluorophenyl)-6-(3-(4-methyl-5-oxo-4,5-dihydro-1,3,4-oxadiazol-2-yl)-5-(trifluoromethyl)-1H-pyrazol-1-yl)nicotinamide;
2-(4-Chloro-phenyl)-3-[1-(4-chloro-phenyl)-5-methyl-1H-pyrazol-3-yl]-propionic acid (FC-2399);
the compounds illustrated in FIGS. 21A-21D, FIG. 22, FIG. 23, and FIGS. 24A-24I;
the compounds listed in Table 1;
a compound of formula (I) 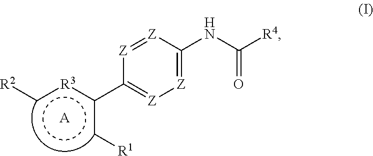
wherein in (I):
ring A is a monocyclic or bicyclic cycloalkyl, heterocyclyl, aryl or heteroaryl ring;
R¹ and R² are independently selected from the group consisting of optionally substituted C₁-C₁₀ alkyl, optionally substituted C₃-C₁₀ cycloalkyl, optionally substituted C₆-C₁₀ aryl, optionally substituted C₄-C₁₀ heterocyclyl, optionally substituted heteroaryl, halogen, —OH, alkoxy, —NH₂, —N(CH₃)₂, trifluoromethyl, —C≡N, —C(═O)OH, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂;
R³ is CH, N, O or S;
each occurrence of Z is independently CH or N; and
R⁴ is optionally substituted C₃-C₁₀ cycloalkyl, optionally substituted C₆-C₁₀ aryl, optionally substituted C₄-C₁₀ heterocyclyl, or optionally substituted heteroaryl.

9. The method of claim 1, wherein the inhibitor is administered as part of a pharmaceutical composition.

10. The method of claim 1, wherein the subject is further administered at least one additional antiviral agent.

11. The method of claim 10, wherein the agent and the inhibitor are co-administered to the subject.

12. The method of claim 11, wherein the agent and the inhibitor are co-formulated.

13. The method of claim 1, wherein the subject is a mammal.

14. The method of claim 13, wherein the mammal is human.

15. A compound of formula (I), or a salt or solvate thereof: 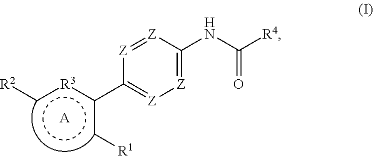
wherein in:
ring A is a monocyclic or bicyclic cycloalkyl, heterocyclyl, aryl or heteroaryl ring;
R¹ and R² are independently selected from the group consisting of optionally substituted C₁-C₁₀ alkyl, optionally substituted C₃-C₁₀ cycloalkyl, optionally substituted C₆-C₁₀ aryl, optionally substituted C₄-C₁₀ heterocyclyl, optionally substituted heteroaryl, halogen, —OH, alkoxy, —NH₂, —N(CH₃)₂, trifluoromethyl, —C≡N, —C(═O)OH, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂;
R³ is CH, N, O or S;
each occurrence of Z is independently CH or N; and
R⁴ is optionally substituted C₃-C₁₀ cycloalkyl, optionally substituted C₆-C₁₀ aryl, optionally substituted C₄-C₁₀ heterocyclyl, or optionally substituted heteroaryl.

16. A pharmaceutical composition comprising at least one compound of claim 15.

17. The composition of claim 16, further comprising at least one additional antiviral agent.

18. The composition of claim 17, wherein the compound and antiviral agent are coformulated.
